(19) United States
(12) Patent Application Publication
(10) Pub. No.: US 2022/0193261 A1
(43) Pub. Date: Jun. 23, 2022

(54) COMPOSITIONS USEFUL FOR TREATMENT OF POMPE DISEASE

(71) Applicants: The Trustees of the University of Pennsylvania, Philadelphia, PA (US); Amicus Therapeutics, Inc., Cranbury, NJ (US)

(72) Inventors: James M. Wilson, Philadelphia, PA (US); Juliette Hordeaux, Philadelphia, PA (US); Hung V. Do, Edmond, OK (US); Russell Gotschall, Doylestown, PA (US); Steven Tuske, Farmingdale, NJ (US)

(21) Appl. No.: 17/606,414

(22) PCT Filed: Apr. 29, 2020
(86) PCT No.: PCT/US2020/030484

§ 371 Date: Oct. 25, 2021

Related U.S. Application Data
(60) Provisional application No. 62/913,401, filed on Oct. 10, 2019.
(60) Provisional application No. 62/840,911, filed on Apr. 30, 2019.

(51) Int. Cl. A61K 48/00; C07K 14/65; C12N 9/26; A61P 3/00

(52) U.S. Cl. CPC A61K 48/005; C07K 14/65; C07K 2319/02; C12Y 302/0102; A61P 3/00; C12N 9/2408

(57) ABSTRACT

A recombinant adeno-associated virus (rAAV) useful for treating type II glycogen storage disease (Pompe) disease is provided. The rAAV comprises an AAV capsid which targets cells of at least one of muscle, heart, kidney, and the central nervous system and which has packaged therein a vector genome comprising a nucleic acid sequence encoding a a chimeric fusion protein comprising a signal peptide and a vIGF2 peptide fused to a human acid-α-glucosidase hGAA780I protein under the control of regulatory sequences which direct its expression. Also provided are methods of making and using this rAAV.

### BACKGROUND OF THE INVENTION

Neurotropic viruses, such as the neurotropic AAV serotypes (e.g. AAV9) have been demonstrated to transduce spinal alpha motor neurons when administered intravenously at high doses in newborn and juvenile animals. This observation led to the recent successful application of AAV9 delivery to treat infants with spinal muscular atrophy, an inherited deficiency of the survival of motor neuron (SMN) protein characterized by selective death of lower motor neurons. In a study involving another neurotropic AAV (AAVhu68), similar results were observed with efficient transduction of spinal cord motor neurons and sensory neurons of dorsal root ganglia after both systemic administration and intrathecal (cerebrospinal fluid) administration (C. Hinderer, et al., Hum Gene Ther. 2018 March; 29(3):285-298). Transduction of DRG neurons was however accompanied by toxicity to those sensory neurons and secondary axonopathy in the spinal cord dorsal tracts. Similar findings were encountered after intravenous and intrathecal delivery of AAV vectors at high doses, irrespective of the capsid serotype or transgene (See, J. Hordeaux, Molecular Therapy: Methods & Clinical Development Vol. 10, pp. 79-88, September 2018).

Pompe disease, also known as type II glycogenosis, is a lysosomal storage disease caused by mutations in the acid-α-glucosidase (GAA) gene leading to glycogen accumulation in the heart (cardiomyopathy), muscles, and motor neurons (neuromuscular disease). In classic infantile Pompe disease, severe GAA activity loss causes multi-system and early-onset glycogen storage, especially within the heart and muscles, and death during the first years from cardiorespiratory failure. Infantile Pompe disease is also characterized by marked glycogen storage within neurons (especially motor neurons) and glial cells. The current standard of care, enzyme replacement therapy (ERT), has suboptimal efficiency to correct muscles and cannot cross the blood-brain barrier, leading to progressive neurologic deterioration in long term survivors of classic infantile Pompe disease. Patients receiving ERT, who live longer due to cardiac correction, reveal a new natural history with a progressive neurologic phenotype. In addition, recombinant human GAA is highly immunogenic and must be dosed in very large quantities due to poor uptake by skeletal muscle.

There are several unmet needs for treatment of Pompe disease, including the need for correction of the CNS component of the disease, the need for improved muscular correction, and the need for an alternative to current ERT that is more efficacious, less immunogenic, and/or more convenient.

### SUMMARY OF THE INVENTION

In certain embodiments, an expression cassette is provided which comprises a nucleic acid sequence encoding a chimeric fusion protein comprising a signal peptide and a vIGF2 peptide fused to a human acid-α-glucosidase (hGAA) comprising at least the active site of hGAA780I under the control of a regulatory sequences which direct its expression, wherein position 780 is based on the numbering of the positions of the amino acid sequence in SEQ ID NO: 3. In certain embodiments, the hGAA comprises at least amino acids 204 to amino acids 890 of SEQ ID NO: 3 (hGAA7800, or a sequence at least 95% identical thereto which has an Ile at position 780. In certain embodiments, the hGAA comprises at least amino acids 204 to amino acids 952 of SEQ ID NO: 3, or a sequence at least 95% identical thereto which has an Ile at position 780. In certain embodiments, the hGAA comprises at least amino acids 123 to amino acids 890 of SEQ ID NO: 3, or a sequence at least 95% identical thereto which has an Ile at position 780. In certain embodiments, the hGAA comprises at least amino acids 70 to amino acids 952 of SEQ ID NO: 3, or a sequence at least 95% identical thereto which has an Ile at position 780. In certain embodiments, the hGAA comprises at least amino acids 70 to amino acids 890 of SEQ ID NO: 3, or a sequence at least 95% identical thereto which has an Ile at position 780. In certain embodiment, the expression cassette further comprises at least two tandem repeats of miR target sequences, wherein the at least two tandem repeats comprise at least a first miRNA target sequence and at least a second miRNA target sequence which may be the same or different and are operably linked 3′ to the sequence encoding the fusion protein.

In certain embodiments, an expression cassette provided herein is carried by a viral vector selected from a recombinant parvovirus, a recombinant lentivirus, a recombinant retrovirus, and a recombinant adenovirus. In certain embodiments, the recombinant parvovirus is a clade F adeno-associated virus, optionally AAVhu68. In certain embodiments, an expression cassette provided herein is carried by a non-viral vector selected from naked DNA, naked RNA, an inorganic particle, a lipid particle, a polymer-based vector, or a chitosan-based formulation.

In certain embodiments, provided herein is a recombinant adeno-associated virus (rAAV) comprising (a) an AAV capsid which targets cells of at least one of muscle, heart, and the central nervous system, and (b) a vector genome packaged in the AAV capsid, the vector genome comprising a nucleic acid sequence encoding a chimeric fusion protein comprising a signal peptide and a vIGF2 peptide fused to a hGAA comprising at least the active site of hGAA780I under the control of a regulatory sequences which direct its expression, wherein position 780 is based on the numbering of the positions of the amino acid sequence in SEQ ID NO: 3. In certain embodiments, the hGAA comprises at least amino acids 204 to amino acids 890 of SEQ ID NO: 3 (hGAA780I), or a sequence at least 95% identical thereto which has an Ile at position 780. In certain embodiments, the hGAA comprises at least amino acids 204 to amino acids 952 of SEQ ID NO: 3, or a sequence at least 95% identical thereto which has an Ile at position 780. In certain embodiments, the hGAA comprises at least amino acids 123 to amino acids 890 of SEQ ID NO: 3, or a sequence at least 95% identical thereto which has an Ile at position 780. In certain embodiments, wherein the hGAA comprises at least amino acids 70 to amino acids 952 of SEQ ID NO: 3, or a sequence at least 95% identical thereto which has an Ile at position 780. In certain embodiments, wherein the hGAA comprises at least amino acids 70 to amino acids 890 of SEQ ID NO: 3, or a sequence at least 95% identical thereto which has an Ile at position 780. In certain embodiments, the rAAV vector genome further comprises least two tandem repeats of dorsal root ganglion (DRG)-specific miR-183 target sequences, wherein the at least two tandem repeats comprise at least a first miRNA target sequence and at least a second miRNA target sequence which may be the same or different and are operably linked 3′ to the sequence encoding the fusion protein.

In certain embodiments, a composition is provided which comprises an expression cassette encoding a hGAA780I fusion protein as described herein and least one of each a pharmaceutically acceptable carrier, an excipient and/or a suspending agent.

In certain embodiments, a composition is provided which includes a rAAV which comprises an expression cassette encoding a hGAA780I fusion protein as described herein and at least one of each a pharmaceutically acceptable carrier, an excipient and/or a suspending agent.

In certain embodiments, a method for treating a patient having Pompe disease and/or for improving cardiac, respiratory and/or skeletal muscle function in a patient having a deficiency in alpha-glucosidase (GAA) is provided. This method comprises delivering to the patient an expression cassette, rAAV, or composition as described herein. The expression cassette, rAAV, or composition may be delivered intravenously and/or via intrathecal, intracisternal or intracerebroventricular administration. Additionally or alternatively, such gene therapy may involve direct delivery to the heart (cardiac), delivery to the lung (intranasal, inhalation, intratracheal), and/or intramuscular injection. One of these may be the sole route of administration of an expression cassette, vector, or composition, or co-administered with other routes of delivery.

A therapeutic regimen for treating a patient having Pompe disease may comprise delivering to the patient an expression cassette, rAAV, or composition as described herein alone, or in combination with a co-therapy, e.g., in combination with one or more of an immunomodulator, a bronchodilator, an acetylcholinesterase inhibitor, respiratory muscle strength training (RMST), enzyme replacement therapy, and/or diaphragmatic pacing therapy.

In certain embodiments, nucleic acid molecules and host cells for production of the expression cassettes and/or a rAAV described herein are provided.

In certain embodiments, use of an expression cassette, rAAV, and/or composition in preparing a medicament is provided.

In certain embodiments, an expression cassette, rAAV, and/or composition suitable for treating a patient having Pompe disease and/or for improving cardiac, respiratory and/or skeletal muscle function in a patient having a deficiency in alpha-glucosidase (GAA) is provided.

Other aspects and advantages of the invention will be readily apparent from the following detailed description of the invention.

### BRIEF DESCRIPTION OF THE DRAWINGS

FIG. 1A and FIG. 1B show hGAA activity in liver of Pompe (−/−) mice four weeks post intravenous administration of various AAVhu68.hGAA having an engineered coding sequence for hGAAV780I under the direction of a CB6 (third column), CAG (fourth column) or UbC promoter (last column). (FIG. 1A) Low dose (1×10¹¹ GC). (FIG. 1B) High dose (1×10¹²).

FIG. 2A and FIG. 2B show hGAA activity in heart of Pompe (−/−) mice four weeks post intravenous administration of various AAVhu68.hGAA having an engineered coding sequence for hGAAV780I under the direction of a CB6 (third column), CAG (fourth column) or UbC promoter (last column). (FIG. 2A) Low dose (1×10¹¹ GC). (FIG. 2B) High dose (1×10¹²).

FIG. 3A and FIG. 3B show hGAA activity in skeletal muscle (quadriceps) of Pompe (−/−) mice four weeks post intravenous administration of various AAVhu68.hGAA having an engineered coding sequence for a hGAAV780I under the direction of a CB6 (third column), CAG (fourth column) or UbC promoter (last column). (FIG. 3A) Low dose (1×10¹¹ GC). (FIG. 3B) High dose (1×10¹²).

FIG. 4A and FIG. 4B show hGAA activity in brain of Pompe (−/−) mice four weeks post intravenous administration of various AAVhu68.hGAA having an engineered coding sequence for a hGAAV780I under the direction of a CB6 (third column), CAG (fourth column) or UbC promoter (last column). (FIG. 4A) Low dose (1×10¹¹ GC). (FIG. 4B) High dose (1×10¹²). The vector expressing under the CB7 activity has lower activity at both doses, while the vectors expressing under the CAG or UbC promoters have comparable activity at the higher dose.

FIG. 5A-FIG. 5H show histology of the heart in Pompe mice (PAS staining showing glycogen storage) four weeks post-delivery of AAVhu68.hGAA. rAAVhu68 vectors containing five different hGAA expression cassettes were generated and assessed. Vehicle control Pompe (−/−) (FIG. 5D) and wildtype (+/+) (FIG. 5A) mice received PBS injections. “hGAA” refers to the reference natural enzyme (hGAAV780) encoded by the wildtype sequence having the native signal peptide (FIG. 5B). “BiP-vIGF2.hGAAco” refers to an engineered coding sequence for the reference hGAAV780 protein containing a deletion of the first 35 AA, and further having a BiP signal peptide, fusion with IGF2 variant with low affinity to insulin receptor (FIG. 5C). “hGAAcoV780I” refers to a hGAAV780I variant encoded by an engineered sequence and containing the native signal peptide (FIG. 5E). “BiP-vIGF2.hGAAcoV780I” refers to the hGAAcoV780I containing a deletion of the first 35 AA, and further having a BiP signal peptide fused with an IGF2 variant with low affinity to insulin receptor and hGAAV780I encoded by the engineered sequence (FIG. 5F). “Sp7.Δ8.hGAAcoV780I” refers to the hGAAV780I variant with a deletion of the first 35 AA encoded by the same engineered sequence as the previous construct but containing sequences encoding a B2 chymotrypsinogen signal peptide in the place of the native signal peptide (FIG. 5G). (FIG. 5H) Blinded histopathology semi-quantitative severity scoring. A board-certified Veterinary Pathologist reviewed the slides in a blinded fashion and established severity scoring based on glycogen storage and autophagy buildup.

FIG. 6A-FIG. 6H show results from histology of quadriceps muscle (PAS stain) in Pompe mice four weeks post-administration of AAVhu68 encoding various hGAA (2.5×10¹³ GC/kg). Control Pompe (−/−) (FIG. 6D) and wildtype (+/+) (FIG. 6A) mice received PBS injections. “hGAA” refers to the reference natural enzyme (hGAAV780) encoded by the wildtype sequence having the native signal peptide (FIG. 6B). “hGAAcoV780I” refers to a hGAAV780I variant encoded by an engineered sequence and containing the native signal peptide (FIG. 6E). “Sp7.Δ8.hGAAcoV780I” refers to the hGAAV780I variant with a deletion of the first 35 AA encoded by the same engineered sequence as the previous construct but containing sequences encoding a B2 chymotrypsinogen signal peptide in the place of the native signal peptide (FIG. 6F). “BiP-vIGF2.hGAAco” refers to the reference hGAAV780 containing a deletion of the first 35 AA, and further having a BiP signal peptide, fusion with IGF2 variant with low affinity to insulin receptor and encoded by an engineered sequence (FIG. 6C). “BiP-vIGF2.hGAAcoV780I” refers to the hGAAV780I containing a deletion of the first 35 AA, and further having a BiP signal peptide fused with an IGF2 variant with low affinity to insulin receptor and hGAAV780I encoded by the engineered sequence (FIG. 6G). (FIG. 6H) Blinded histopathology semi-quantitative severity scoring. A board-certified Veterinary Pathologist reviewed the slides in a blinded fashion and established severity scoring based on glycogen storage and autophagy buildup. A score of 0 means no lesion; 1 means less than 9% of muscle fibers affected by storage on average; 2 means 10 to 49%; 3 means 50 to 75% and 4 means 76 to 100%.

FIG. 7A-FIG. 7H show results from histology of quadriceps muscle (Periodic acid-Schiff (PAS) stain) from Pompe mice four weeks post-administration of AAVhu68 encoding various hGAA at 2.5×10¹² GC/Kg (i.e. a 10-fold lower dose than in FIG. 6A-FIG. 6H). Control Pompe (−/−) (FIG. 7D) and wildtype (+/+) (FIG. 7A) mice received PBS injections. “hGAA” refers to the reference natural enzyme (hGAAV780) encoded by the wildtype sequence having the native signal peptide (FIG. 7B). “hGAAcoV780I” refers to a hGAAV780I variant encoded by an engineered sequence and containing the native signal peptide (FIG. 7E). “Sp7.Δ8.hGAAcoV780I” refers to the hGAAV780I variant with a deletion of the first 35 AA encoded by the same engineered sequence as the previous construct but containing sequences encoding a B2 chymotrypsinogen signal peptide in the place of the native signal peptide (FIG. 7F). “BiP-vIGF2.hGAAco” refers to the reference hGAAV780 containing a deletion of the first 35 AA, and further having a BiP signal peptide, fusion with IGF2 variant with low affinity to insulin receptor and encoded by an engineered sequence (FIG. 7C). “BiP-vIGF2.hGAAcoV780I” refers to the hGAAV780I containing a deletion of the first 35 AA, and further having a BiP signal peptide fused with an IGF2 variant with low affinity to insulin receptor and hGAAV780I encoded by the engineered sequence (FIG. 7G). (FIG. 7H) Blinded histopathology semi-quantitative severity scoring. A board-certified Veterinary Pathologist reviewed the slides in a blinded fashion and established severity scoring based on glycogen storage and autophagy buildup. A score of 0 means no lesion; 1 means less than 9% of muscle fibers affected by storage on average; 2 means 10 to 49%; 3 means 50 to 75% and 4 means 76 to 100%.

FIG. 8 shows results from histology of the spinal cord (PAS and luxol fast blue stain) from Pompe mice four weeks post administration (2.5×10¹² GC/kg) of AAVhu68 having a sequence encoding the native hGAA or an hGAAV780I containing a deletion of the first 35 AA, and further having a BiP signal peptide fused with an IGF2 variant with low affinity to insulin receptor and hGAAV780I encoded by the engineered sequence (“BiP-vIGF2.hGAAcoV780I”). Blinded histopathology semi-quantitative severity scoring was performed on spinal cord sections.

FIG. 9A-FIG. 9C show hGAA activity in plasma and binding to IGF2/CI-MPR. Pompe mice were administered vectors encoding a wildtype hGAA or BiP-vIGF2.hGAA at low dose (2.5×10¹² GC). (FIG. 9A, FIG. 9B) Four weeks post intravenous administration high levels of wildtype and engineered hGAA activity were detected in plasma. (FIG. 9C) Engineered hGAA binds efficiently to CI-MPR.

FIG. 10 shows glycogen clearance and resolution of autophagic buildup in Pompe mice four weeks post administration of AAVhu68 constructs at a dose of 2.5×10¹² GC/Kg (LD). Paraffin sections of gastrocnemius muscles stained with DAPI and anti-LC3B antibodies.

FIG. 11 shows a schematic for a BiP-vIGF2.hGAAcoV780I.4×miR183 construct.

FIG. 12 shows glycogen storage (PAS, luxol blue stain) in the brainstem of Pompe mice four weeks post-intravenous administration of AAVhu68.CAG.BiP-vIGF2.hGAAcoV780I.4XmiR183 (containing four copies of a drg-detargetting sequence, miR183) at a high dose (HD: 2.5×10¹³ GC/kg) or a low dose (LD: 2.5×10¹² GC/kg). Arrows show PAS positive storage within neurons.

FIG. 13 shows glycogen storage (PAS, luxol blue stain) in the spinal cord of Pompe mice four weeks post intravenous administration of AAVhu68.CAG.BiP-vIGF2.hGAAcoV780I.4XmiR183 at a high dose (HD: 2.5×10¹³ GC/kg) or a low dose (LD: 2.5×10¹² GC/kg). Arrows show PAS positive storage within neurons.

FIG. 14 shows glycogen storage (PAS stain) in the quadriceps muscle of Pompe mice four weeks post intravenous administration of AAVhu68.CAG.BiP-vIGF2.hGAAcoV780I.4XmiR183 at a high dose (HD: 2.5×10¹³ GC/kg) or a low dose (LD: 2.5×10¹² GC/kg).

FIG. 15 shows glycogen storage (PAS stain) in the heart of Pompe mice four weeks post intravenous administration of AAVhu68.CAG.BiP-vIGF2.hGAAcoV780I.4XmiR183 at a high dose (HD: 2.5×10¹³ GC/kg) or a low dose (LD: 2.5×10¹² GC/kg).

FIG. 16 shows expression the autophagic vacuole marker LC3b in quadriceps muscle of Pompe mice four weeks post intravenous administration of AAVhu68.CAG.BiP-vIGF2.hGAAcoV780I.4XmiR183 at a high dose (HD: 2.5×10¹³ GC/kg) or a low dose (LD: 2.5×10¹² GC/kg).

FIG. 17 shows representative images of hGAA expression (immunohistochemistry for hGAA) in cervical DRG of rhesus macaques 35 days after the ICM administration of AAVhu68.CAG.BiP-vIGF2.hGAAcoV780I (left) or AAVhu68.CAG.BiP-vIGF2.hGAAcoV780I.4XmiR183 (right) at a high dose of 3e13 GC.

FIG. 18 show representative images of hGAA expression (immunohistochemistry to hGAA) in lumbar DRG of rhesus macaques 35 days after the ICM administration of AAVhu68.CAG.BiP-vIGF2.hGAAcoV780I (left) or AAVhu68.CAG.BiP-vIGF2.hGAAcoV780I.4XmiR183 (right) at a high dose of 3e13 GC.

FIG. 19 shows representative images of hGAA expression (immunohistochemistry to hGAA) in the spinal cord lower motor neurons of rhesus macaques 35 days after the ICM administration of AAVhu68.CAG.BiP-vIGF2.hGAAcoV780I (left) or AAVhu68.CAG.BiP-vIGF2.hGAAcoV780I.4XmiR183 (right) at a high dose of 3e13 GC.

FIG. 20 shows representative images of hGAA expression (immunohistochemistry to hGAA) in the heart of rhesus macaques 35 days after the ICM administration of AAVhu68.CAG.BiP-vIGF2.hGAAcoV780I (left) or AAVhu68.CAG.BiP-vIGF2.hGAAcoV780I.4XmiR183 (right) at a high dose of 3e13 GC.

FIG. 21A-FIG. 21C show histopathological scoring of DRG neuronal degeneration and inflammatory cell infiltration in the DRG of cervical segment (FIG. 21A), thoracic segment (FIG. 21B), and lumbar segment (FIG. 21C) in rhesus macaques 35 days after ICM administration of AAVhu68.CAG.BiP-vIGF2.hGAAcoV780I or AAVhu68.CAG.BiP-vIGF2.hGAAcoV780I.4XmiR183 at a high dose 3×10¹³ GCs. AAVhu68 vectors were delivered in a total volume of 1 mL of sterile artificial CSF (vehicle) injected into the cisterna magna, under fluoroscopic guidance as previously described (Katz et al., Hum Gene Ther. Methods, 2018, 29:212-9). A board-certified Veterinary Pathologist who was blinded to the vector group established severity grades defined with 0 as absence of lesion, 1 as minimal (<10%), 2 mild (10-25%), 3 moderate (25-50%), 4 marked (50-95%), and 5 severe (>95%). Each data point represents one DRG. A minimal of five DRG per segment and per animal were scored.

FIG. 22A-FIG. 22C show AST levels (FIG. 22A), ALT levels (FIG. 22B), and platelet counts (FIG. 22C) for rhesus macaques following ICM administration of AAVhu68.CAG.BiP-vIGF2.hGAAcoV780I or AAVhu68.CAG.BiP-vIGF2.hGAAcoV780I.4XmiR183 at a high dose of 3e13 GC.

FIG. 23 shows plasma hGAA activity levels in NHP administered (ICM) AAVhu68.CAG.BiP-vIGF2.hGAAcoV780I or AAVhu68.CAG.BiP-vIGF2.hGAAcoV780I.4XmiR183 at a high dose of 3e13 GC at days 0-35 post injection.

FIG. 24A-FIG. 24G show results from nerve conduction velocity tests at baseline and day 35 for NHP administered (ICM, 3e13 GC) AAVhu68.CAG.BiP-vIGF2.hGAAcoV780I or AAVhu68.CAG.BiP-vIGF2.hGAAcoV780I.4XmiR183.

FIG. 25A and FIG. 25B show body weight longitudinal follow-up from vector injection (day 0) to 180 days post-injection in Pompe mice that were treated at an advanced stage of disease at 7 months of age and were already symptomatic at baseline. They received AAVhu68.CAG.BiP-vIGF2.hGAAcoV780I using via alternative routes of administration and dose levels: intracerebroventricular (ICV) at high dose (HD) (1e11 GC) or low dose (LD) (5e10 GC), intravenous (IV) at HD (5e13 GC/Kg) or LD (1e13 GC/Kg), and a combination of ICV and IV at low doses or high doses. Mean value and standard deviation are depicted. Statistical analysis at each time point is performed by Wilcoxon-Mann-Whitney test between KO PBS control groups and the other groups. *p<0.05; **p<0.01

FIG. 26A and FIG. 26B show grip strength relative to body weight longitudinal follow-up from vector injection (day 0) to 180 days post-injection in Pompe mice that were treated at an advanced stage of disease at 7 months of age and were already symptomatic at baseline. (FIG. 26A) Mice received AAVhu68.CAG.BiP-vIGF2.hGAAcoV780I via alternative routes of administration and dose levels: intracerebroventricular (ICV) at high dose (ICV HD: 1e11 GC), intravenous (IV) at high dose (IV HD: 5e13 GC/Kg), and combinations of ICV and IV high doses and ICV and IV low doses. Grip strength was measured at various timepoints using a grip strength meter (IITC Life Science). The transducer in the Grip Strength Meter is connected to a wire mesh grid connected to an anodized base plate. The animal is held by its tail and is gently passed over the mesh until it grasps the grid with its four paws. Three grip force measures were made, and the average of these readings represents the animal's grip force at that particular time. (FIG. 26B) Results from day 180 showing incremental benefit of IV+ICV HD versus IV HD. Values are normalized by animal body weight. N=4 males and 4 females per group. Statistical analysis at each time point was determined by 1-way ANOVA (FIG. 26A) or 2-way ANOVA (FIG. 26B), post-hoc multiple comparison test compared to KO PBS control group. *p<0.05, **p<0.01, ***p<0.001

FIG. 27A and FIG. 27B show results of plethysmography with Pompe mice administered AAVhu68.CAG.BiP-vIGF2.hGAAcoV780I vector IV, ICV, or IV and ICV (dual route). (FIG. 27A) 5% CO2 challenge. (FIG. 27B) 7% CO2 challenge.

FIG. 28 shows glycogen storage in the quadriceps, heart, and spinal cord of post-symptomatic Pompe mice following high dose (HD: 1e11 GC) or low dose (LD: 5e10 GC) ICV administration of AAVhu68.CAG.BiP-vIGF2.hGAAcoV780I.

FIG. 29 shows glycogen storage in the quadriceps, heart, and spinal cord of post-symptomatic Pompe mice following high dose (HD: 5e13 GC/Kg) or low dose (LD: 1e13 GC/Kg) IV administration of AAVhu68.CAG.BiP-vIGF2.hGAAcoV780I.

FIG. 30A-FIG. 30C show hGAA activity in plasma of Pompe mice administered AAVhu68.CAG.BiP-vIGF2.hGAAcoV780I vector IV, ICV, or IV and ICV (dual route) at day 30 (FIG. 30A), day 60 (FIG. 30B), and day 90 (FIG. 30C).

FIG. 31 shows a study design for evaluation of single (IV or ICM) and dual routes (IV+ICM) of administration in NHP.

FIG. 32A-FIG. 32H show detection of hGAA and hGAA activity in plasma and CSF of NHP following IV or ICM administration of AAVhu68.CAG.BiP-vIGF2.hGAAcoV780I.

FIG. 33A-FIG. 33F show histopathological scoring of DRG neuronal degeneration and inflammatory cell infiltration (FIG. 33A-FIG. 33C) and spinal cord axonopathy (FIG. 33D-FIG. 33F) of rhesus macaques following IV (1e13 GC/Kg or 5e13 GC/Kg) or ICM (1e13 GC or 3e13 GC) administration of AAVhu68.CAG.BiP-vIGF2.hGAAcoV780I. A board-certified Veterinary Pathologist who was blinded to the vector group established severity grades defined with 0 as absence of lesion, 1 as minimal (<10%), 2 mild (10-25%), 3 moderate (25-50%), 4 marked (50-95%), and 5 severe (>95%).

FIG. 34 shows representative images of hGAA expression (immunohistochemistry to hGAA) in the quadriceps, heart, and spinal cord of rhesus macaques following low dose (IV— 1e13 GC/Kg, ICM—1e13 GC) administration of AAVhu68.CAG.BiP-vIGF2.hGAAcoV780I.

### DETAILED DESCRIPTION OF THE INVENTION

Compositions are provided for delivering a fusion protein comprising a signal peptide and a vIGF2 peptide fused to at least the active portion of a hGAA780I enzyme to patients having Pompe disease. Methods of making and using the same are described herein, including regimens for treating patients with these compositions.

As used herein, the term “Pompe disease,” also referred to as maltase deficiency, glycogen storage disease type II (GSDII), or glycogenosis type II, is intended to refer to a genetic lysosomal storage disorder characterized by a total absence or a partial deficiency in the lysosomal enzyme acid α-glucosidase (GAA) caused by mutations in the GAA gene, which codes for the acid α-glucosidase. The term includes but is not limited to early and late onset forms of the disease, including but not limited to infantile, juvenile, and adult-onset Pompe disease.

It will be understood that the Greek letter “alpha” and the symbol “a” are used interchangeably throughout this specification. Similarly, the Greek letter “delta” and “Δ” are used interchangeably throughout this specification.

As used herein, the term “acid α-glucosidase” or “GAA” refers to a lysosomal enzyme which hydrolyzes α-1,4 linkages between the D-glucose units of glycogen, maltose, and isomaltose. Alternative names include but are not limited to lysosomal α-glucosidase (EC:3.2.1.20); glucoamylase; 1,4-α-D-glucan glucohydrolase; amyloglucosidase; gamma-amylase and exo-1,4-α-glucosidase. Human acid α-glucosidase is encoded by the GAA gene (National Centre for Biotechnology Information (NCBI) Gene ID 2548), which has been mapped to the long arm of chromosome 17 (location 17q25.2-q25.3). The conserved hexapeptide WIDMNE at amino acid residues 516-521 is required for activity of the acid α-glucosidase protein. The term “hGAA” refers to a coding sequence for a human GAA.

As used herein, a “rAAV.hGAA” refers to a rAAV having an AAV capsid which has packaged therein a vector genome containing, at a minimum, a coding sequence for a GAA enzyme (e.g., a 780I variant, a fusion protein comprising a signal peptide and a vIGF2 peptide fused to at least the active portion of a hGAA780I enzyme). rAAVhu68.hGAA or rAAVhu68.hGAA refers to a rAAV in which the AAV capsid is an AAVhu68 capsid, which is defined herein.

With reference to the numbering of the full-length hGAA, there is a signal peptide at amino acid positions 1 to 27. Additionally, the enzyme has been associated with multiple mature proteins, i.e., a mature protein at amino acid positions 70 to 952, a 76 kD mature protein located at amino acid positions 123 to 952, and a 70 kD mature protein at amino acid 204 to amino 952. The “active catalytic site” comprises the hexapeptide WIDMNE (amino acid residues 516-521 of SEQ ID NO: 3). In certain embodiments, a longer fragment may be selected, e.g., positions 516 to 616. Other active sites include ligand binding sites, which may be located at one or more of positions 376, 404, 405, 441, 481, 516, 518, 519, 600, 613, 616, 649, 674.

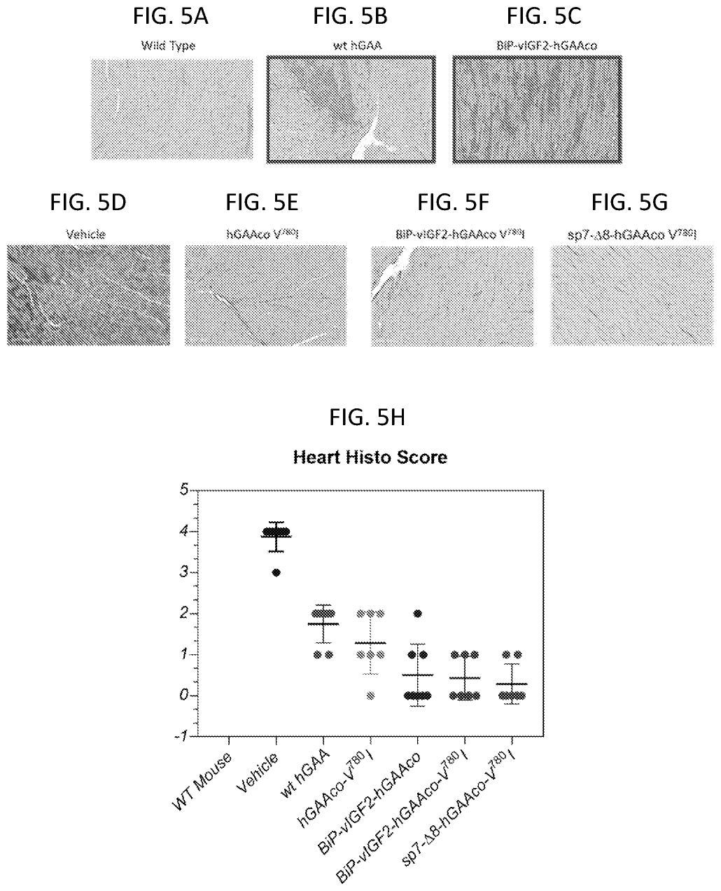

Unless otherwise specified, the term “hGAA780I” or “hGAAV780I” refers to the full-length pre-pro-protein having the amino acid sequence reproduced in SEQ ID NO: 3. In some instances, the term hGAAco780I or hGAAcoV780I is used to refer to an engineered sequence encoding hGAA780I. As compared to the hGAA reference protein described in the preceding paragraph, hGAA780I has an isoleucine (Ile or I) at position 780 where the reference hGAA contains a valine (Val or V). This hGAA780I has been unexpectedly found to have a better effect and improved safety profile than the hGAA sequence having a valine at position 780 (hGAAV780), which has been widely described in the literature as the “reference sequence”. For example, as can be seen in FIG. 5A-FIG. 5H, the hGAAV780 reference sequence induces toxicity (fibrosing cardiomyositis) not seen as the same dose with the hGAA780I enzyme. Thus, use of the hGAA780I may reduce or eliminate fibrosing cardiomyositis in patients receiving therapy with a hGAA. The location of the hGAA signal peptide, mature protein, active catalytic sites, and binding sites may be determined based on the analogous location in the hGAA780I reproduced in SEQ ID NO: 3, i.e., signal peptide at amino acid positions 1 to 27; mature protein at amino acid positions 70 to 952; a 76 kD mature protein located at amino acid positions 123 to 952, and a 70 kD mature protein at amino acid 204 to amino 952; “active catalytic site” comprising hexapeptide WIDMNE (SEQ ID NO: 61) at amino acid residues 516-521; other active sites include ligand binding sites, which may be located at one or more of positions 376, 404 . . . 405, 441, 481, 516, 518 . . . 519, 600, 613, 616, 649, 674.

In certain embodiments, a hGAA780I may be selected which has a sequence which is at least 95% identical to the hGAA780I, at least 97% identical to the hGAA780I, or at least 99% identical to the hGAA780I of SEQ ID NO: 3. In certain embodiments, provided is sequence which is at least 95%, at least 97%, or at least 99 identity to a mature hGAA780I protein of SEQ ID NO: 3. In certain embodiments, the sequence having at least 95% to at least 99% identity to the hGAA780I has the sequence for the active catalytic site retained without any change. In certain embodiments, the sequence having at least 95% to at least 99% identity to the hGAA780I to SEQ ID NO: 3 is characterized by having an improved biological effect and better safety profile than the reference hGAAV780 when tested in appropriate animal models. In certain embodiments, a GAA activity assay may be performed as previously described (see, e.g., J. Hordeaux, et. al., Acta Neuropathological Communications, (2107) 5: 66) or using other suitable methods. In certain embodiments, the hGAA780I enzyme contains modifications in other positions in the hGAA amino acid sequence. Examples of mutants may include, e.g., those described in U.S. Pat. No. 9,920,307. In certain embodiments, such mutant hGAA780I may retain at a minimum, the active catalytic site: WIDMNE (SEQ ID NO: 61) and amino acids in the region of 780I as described below.

In certain embodiments, a novel hGAA780I fusion protein is provided which comprises a leader peptide other than the native hGAA signal peptide. In certain embodiments, such an exogenous leader peptide is preferably of human origin and may include, e.g., an IL-2 leader peptide. Particular exogenous signal peptides workable in the certain embodiments include amino acids 1-20 from chymotrypsinogen B2, the signal peptide of human alpha-1-antitrypsin, amino acids 1-25 from iduronate-2-sulphatase, and amino acids 1-23 from protease CI inhibitor. See, e.g., WO2018046774. Other signal/leader peptides may be natively found in an immunoglobulin (e.g., IgG), a cytokine (e.g., IL-2, IL12, IL18, or the like), insulin, albumin, β-glucuronidase, alkaline protease or the fibronectin secretory signal peptides, amongst others. See, also, e.g., signalpeptide.de/index.php?m=listspdb_mammalia.

Such a chimeric hGAA780I may have the exogenous leader in the place of the entire 27 aa native signal peptide. Optionally, an N-terminal truncation of the hGAA780I enzyme may lack only a portion of the signal peptide (e.g., a deletion of about 2 to about 25 amino acids, or values therebetween), the entire signal peptide, or a fragment longer than the signal peptide (e.g., up to amino acids 70 based on the numbering of SEQ ID NO: 3. Optionally, such an enzyme may contain a C-terminal truncation of about 5, 10, 15, or 20 amino acids in length.

In certain embodiments, a novel fusion protein is provided which comprises the mature hGAA780I protein (aa 70 to 952), the mature 70 kD protein (aa 123 to aa 952), or the mature 76 kD protein (aa 204 to 952) bound to a fusion partner. Optionally, the fusion protein further comprises a signal peptide which is non-native to hGAA. Further optionally, one of these embodiments may further contain a C-terminal truncation of about 5, 10, 15, or 20 amino acids in length.

In certain embodiments, a fusion protein comprising the hGAA780I protein comprises at least amino acids 204 to amino acids 890 of SEQ ID NO: 3 (hGAA780I), or a sequence at least 95% identical thereto which has an Ile at position 780. In certain embodiments, a hGAA780I protein comprises at least amino acids 204 to amino acids 952 of SEQ ID NO: 3 or a sequence at least 95% identical thereto which has an Ile at position 780. In certain embodiments, a hGAA780I protein comprises at least amino acids 123 to amino acids 890 of SEQ ID NO: 3 or a sequence at least 95% identical thereto which has an Ile at position 780. In certain embodiments, the hGAA780I enzyme comprises at least amino acids 70 to amino acids 952 of SEQ ID NO: 3 or a sequence at least 95% identical thereto which has an Ile at position 780. In certain embodiments, the hGAA780I protein comprises at least amino acids 70 to amino acids 890 of SEQ ID NO: 3, or a sequence at least 95% identical thereto which has an Ile at position 780.

In certain embodiments, the fusion protein comprises the signal and leader sequences and hGAA780I sequence having at least 95% identity, at least 97% identity, or at least 99% identity to SEQ ID NO: 7, has no changes in the active site and/or no changes in the amino acids 3 to 12 amino acids N-terminus and/or C-terminus to the active site. In preferred embodiments, an engineered hGAA expression cassette encodes at least the human hGAA780I fragment of: T-Val(V)-P-Ile (780I)-Glu(E)-Ala(A)-Leu(L) (SEQ ID NO: 62). In certain embodiments, an engineered hGAA expression cassette encodes a longer human hGAA780I fragment: Gln (Q)-T-V-P-780I-E-A-L-Gly (G) (SEQ ID NO: 63). In certain embodiments, an engineered hGAA expression cassette encodes a fragment corresponding to at least: PLGT-Trp (W)-Tyr (Y)-Asp (D)-LQTVP-780I-EALG-(Ser or S)-L-PPPPAA sequence (SEQ ID NO: 64). Similarly, in preferred embodiments, there are no amino acid changes in the active binding site (aa 518 to 521 of SEQ ID NO: 3). In certain embodiments, the binding sites at positions 600, 616, and/or 674 remain unchanged. In certain embodiments, a fusion protein comprises a signal peptide, an optional vIGF+2GS extension, an optional ER proteolytic peptide, and the hGAA780I variant with a deletion of first 35 amino acids of hGAA (i.e., lacking the native signal peptide and amino acids 28 to 35).

In certain embodiments, a secreted engineered GAA is provided, which comprises a BiP signal peptide, an IGF2+2GS extension and amino acids 61 to 952 of hGAA 780I (with a deletion of amino acids 1 to 60 of hGAA780I). In certain embodiments, provided herein is a fusion protein comprising SEQ ID NO: 6, or a sequence at least 95% identical thereto. In certain embodiments, the fusion protein is encoded by SEQ ID NO: 7, or a sequence at least 95% identical thereto. In certain embodiments, the fusion protein comprises a sequence of SEQ ID NO: 4, or a sequence at least 95% identical thereto. In certain embodiments, the fusion protein comprises a sequence of SEQ ID NO: 5, or a sequence at least 95% identical thereto.

Components of Fusion Proteins Provided Herein are Further Described Below. Peptides that Bind CI-MPR

Provided herein are peptides that bind CI-MPR (e.g., vIGF2 peptides). Fusion proteins comprising such peptides and a hGAA780I protein, when expressed from a gene therapy vector, target the hGAA780I to the cells where it is needed, increase cellular uptake by such cells and target the therapeutic protein to a subcellular location (e.g., a lysosome). In some embodiments, the peptide is fused to the N-terminus of the hGAA780I protein. In some embodiments, the peptide is fused to the C-terminus of the hGAA780I protein. In some embodiments, the peptide is a vIGF2 peptide. Some vIGF2 peptides maintain high affinity binding to CI-MPR while their affinity for IGF1 receptor, insulin receptor, and IGF binding proteins (IGFBP) is decreased or eliminated. Thus, some variant IGF2 peptides are substantially more selective and have reduced safety risks compared to wildtype IGF2. vIGF2 peptides herein include those having the amino acid sequence of SEQ ID NO: 46. Variant IGF2 peptides further include those with variant amino acids at positions 6, 26, 27, 43, 48, 49, 50, 54, 55, or 65 compared to wildtype IGF2 (SEQ ID NO: 34). In some embodiments, the vIGF2 peptide has a sequence having one or more substitutions from the group consisting of E6R, F26S, Y27L, V43L, F48T, R49S, S50I, A54R, L55R, and K65R. In some embodiments, the vIGF2 peptide has a sequence having a substitution of E6R. In some embodiments, the vIGF2 peptide has a sequence having a substitution of F26S. In some embodiments, the vIGF2 peptide has a sequence having a substitution of Y27L. In some embodiments, the vIGF2 peptide has a sequence having a substitution of V43L. In some embodiments, the vIGF2 peptide has a sequence having a substitution of F48T. In some embodiments, the vIGF2 peptide has a sequence having a substitution of R495. In some embodiments, the vIGF2 peptide has a sequence having a substitution of S50I. In some embodiments, the vIGF2 peptide has a sequence having a substitution of A54R. In some embodiments, the vIGF2 peptide has a sequence having a substitution of L55R. In some embodiments, the vIGF2 peptide has a sequence having a substitution of K65R. In some embodiments, the vIGF2 peptide has a sequence having a substitution of E6R, F26S, Y27L, V43L, F48T, R495, S50I, A54R, and L55R. In some embodiments, the vIGF2 peptide has an N-terminal deletion. In some embodiments, the vIGF2 peptide has an N-terminal deletion of one amino acid. In some embodiments, the vIGF2 peptide has an N-terminal deletion of two amino acids. In some embodiments, the vIGF2 peptide has an N-terminal deletion of three amino acids. In some embodiments, the vIGF2 peptide has an N-terminal deletion of four amino acids. In some embodiments, the vIGF2 peptide has an N-terminal deletion of four amino acids and a substitution of E6R, Y27L, and K65R. In some embodiments, the vIGF2 peptide has an N-terminal deletion of four amino acids and a substitution of E6R and Y27L. In some embodiments, the vIGF2 peptide has an N-terminal deletion of five amino acids. In some embodiments, the vIGF2 peptide has an N-terminal deletion of six amino acids. In some embodiments, the vIGF2 peptide has an N-terminal deletion of seven amino acids. In some embodiments, the vIGF2 peptide has an N-terminal deletion of seven amino acids and a substitution of Y27L and K65R.

IGF2 Amino Acid Sequences
(variant residues are underlined)

| | | SEQ |
|---|---|---|
| Peptide | Sequence | ID NO: |
| Wildtype | AYRPSETLCGGELVDTLQFVCGDRGFYFS | 32 |
| | RPASRVSRRSRGIVEECCFRSCDLALLET | |
| | YCATPAKSE | |
| F26S | AYRPSETLCGGELVDTLQFVCGDRGFYFS | 33 |
| | RPASRVSRRSRGIVEECCFRSCDLALLET | |
| | YCATPAKSE | |
| Y27L | AYRPSETLCGGELVDTLQFVCGDRGFLFS | 34 |
| | RPASRVSRRSRGIVEECCFRSCDLALLET | |
| | YCATPAKSE | |
| V43L | AYRPSETLCGGELVDTLQFVCGDRGFYFS | 35 |
| | RPASRVSRRSRGILEECCFRSCDLALLET | |
| | YCATPAKSE | |
| F48T | AYRPSETLCGGELVDTLQFVCGDRGFYFS | 36 |
| | RPASRVSRRSRGIVEECCTRSCDLALLET | |
| | YCATPAKSE | |
| R49S | AYRPSETLCGGELVDTLQFVCGDRGFYFS | 37 |
| | RPASRVSRRSRGIVEECCFSSCDLALLET | |
| | YCATPAKSE | |
| S50I | AYRPSETLCGGELVDTLQFVCGDRGFYFS | 38 |
| | RPASRVSRRSRGIVEECCFRICDLALLET | |
| | YCATPAKSE | |
| A54R | AYRPSETLCGGELVDTLQFVCGDRGFYFS | 39 |
| | RPASRVSRRSRGIVEECCFRSCDLRLLET | |
| | YCATPAKSE | |
| L55R | AYRPSETLCGGELVDTLQFVCGDRGFYFS | 40 |
| | RPASRVSRRSRGIVEECCFRSCDLARLET | |
| | YCATPAKSE | |
| F26S, Y27L, | AYRPSETLCGGELVDTLQFVCGDRGSLFS | 41 |
| V43L, F48T, | RPASRVSRRSRGILEECCTSICDLRRLET | |
| R49S, S50I, | YCATPAKSE | |
| A54R, L55R | | |
| Δ1-6, Y27L, | TLCGGELVDTLQFVCGDRGFLFSRPASRV | 42 |
| K65R | SRRSRGIVEECCFRSCDLALLETYCATPA | |
| | RSE | |
| Δ1-7, Y27L, | LCGGELVDTLQFVCGDRGFLFSRPASRVS | 43 |
| K65R | RRSRGIVEECCFRSCDLALLETYCATPAR | |
| | SE | |
| Δ1-4, E6R, | SRTLCGGELVDTLQFVCGDRGFLFSRPAS | 44 |
| Y27L, K65R | RVSRRSRGIVEECCFRSCDLALLETYCAT | |
| | PARSE | |
| Δ1-4, E6R, | SRTLCGGELVDTLQFVCGDRGFLFSRPAS | 45 |
| Y27L | RVSRRSRGIVEECCFRSCDLALLETYCAT | |
| | PAKSE | |
| E6R | AYRPSRTLCGGELVDTLQFVCGDRGFYFS | 46 |
| | RPASRVSRRSRGIVEECCFRSCDLALLET | |
| | YCATPAKSE | |

IGF2 DNA Coding Sequences

| | | SEQ |
|---|---|---|
| Peptide | DNA Sequence | ID NO |
| Mature WT | GCTTACCGCCCCAGTGAGACCCTGTGCGG | 47 |
| IGF2 | CGGGGAGCTGGTGGACACCCTCCAGTTCG | |
| | TCTGTGGGGACCGCGGCTTCTACTTCAGC | |
| | AGGCCCGCAAGCCGTGTGAGCCGTCGCAG | |
| | CCGTGGCATCGTTGAGGAGTGCTGTTTCC | |
| | GCAGCTGTGACCTGGCCCTCCTGGAGACG | |
| | TACTGTGCTACCCCCGCCAAGTCCGAG | |
| vIGF2 Δ1-4, | TCTAGAACACTGTGCGGAGGGGAGCTTGT | 48 |
| E6R, Y27L, | AGACACTCTTCAGTTCGTGTGTGGAGATC | |
| K65R | GCGGGTTCCTCTTCTCTCGCCCCGCTTCC | |
| | AGAGTTTCACGGAGGTCTAGGGGTATAGT | |
| | AGAGGAGTGTTGTTTCAGGTCCTGTGACT | |
| | TGGCGCTCCTCGAGACCTATTGCGCGACG | |
| | CCAGCCAGGTCCGAA | |

### Signal Peptides

Compositions provided herein, in some embodiments, further comprise a signal peptide, which improves secretion of hGAA780I from the cell transduced with the gene therapy construct. The signal peptide in some embodiments improves protein processing of therapeutic proteins, and facilitates translocation of the nascent polypeptide-ribosome complex to the ER and ensuring proper co-translational and post-translational modifications. In some embodiments, the signal peptide is located (i) in an upstream position of the signal translation initiation sequence, (ii) in between the translation initiation sequence and the therapeutic protein, or (iii) a downstream position of the therapeutic protein. Signal peptides useful in gene therapy constructs include but are not limited to binding immunoglobulin protein (BiP) signal peptide from the family of HSP70 proteins (e.g., HSPA5, heat shock protein family A member 5) and Gaussia signal peptides, and variants thereof. These signal peptides have ultrahigh affinity to the signal recognition particle. Examples of BiP and Gaussia amino acid sequences are provided in the table below. In some embodiments, the signal peptide has an amino acid sequence that is at least 90% identical to a sequence selected from the group consisting of SEQ ID Nos: 49-53. In some embodiments, the signal peptide differs from a sequence selected from the group consisting of SEQ ID Nos: 49-53 by 5 or fewer, 4 or fewer, 3 or fewer, 2 or fewer, or 1 amino acid(s).

Signal Peptide Sequences

| Signal | | SEQ |
|---|---|---|
| Peptide | Amino Acid Sequence | ID NO: |
| Native human | MKLSLVAAMLLLLSAARA | 49 |
| BiP | | |
| Modified BiP-1 | MKLSLVAAMLLLLSLVAAMLLLLSAARA | 50 |
| Modified BiP-2 | MKLSLVAAMLLLLWVALLLLSAARA | 51 |
| Modified BiP-3 | MKLSLVAAMLLLLSLVALLLLSAARA | 52 |
| Modified BiP-4 | MKLSLVAAMLLLLALVALLLLSAARA | 53 |
| Gaussia | MGVKVLFALICIAVAEA | 54 |

The Gaussia signal peptide is derived from the luciferase from Gaussia princeps and directs increased protein synthesis and secretion of therapeutic proteins fused to this signal peptide. In some embodiments, the Gaussia signal peptide has an amino acid sequence that is at least 90% identical to SEQ ID NO: 54. In some embodiments, the signal peptide differs from SEQ ID NO: 54 by 5 or fewer, 4 or fewer, 3 or fewer, 2 or fewer, or 1 amino acid(s).

### Linkers

Compositions provided herein, in some embodiments, comprise a linker between the targeting peptide and the therapeutic protein. Such linkers, in some embodiments, maintain correct spacing and mitigate steric clash between the vIGF2 peptide and the therapeutic protein. Linkers, in some embodiments, comprise repeated glycine residues, repeated glycine-serine residues, and combinations thereof. In some embodiments, the linker consists of 5-20 amino acids, 5-15 amino acids, 5-10 amino acids, 8-12 amino acids, or about 5, 6, 7, 8, 9, 10, 11, 12 or 13 amino acids. Suitable linkers include but are not limited to those provided in the following table:

Linker Sequences

| Sequence | SEQ ID NO: |
|---|---|
| GGGGSGGGG | 55 |
| GGGGS | 56 |
| GGGSGGGGS | 57 |
| GGGGSGGGS | 58 |
| GGSGSGSTS | 59 |
| GGGGSGGGGS | 60 |

Throughout this specification, various expression cassettes, vector genomes, vectors, and, compositions, are described as containing a hGAA780I coding sequence or a hGAA780I protein or fusion protein. It will be understood that, unless otherwise specified, any of the engineered hGAA780I proteins, including N-terminal truncation, C-terminal truncations, and fusion proteins such as those described herein, or coding sequences therefor, may be similarly engineered into expression cassettes, vector genomes, vectors, and compositions.

Suitably, an expression cassette is provided which comprises the nucleic acid sequences described herein.

### Expression Cassette

As used herein, an “expression cassette” refers to a nucleic acid molecule which comprises a nucleic acid sequence encoding a functional gene product operably linked to regulatory sequences which direct its expression in a target cell (e.g., a hGAA780I fusion protein coding sequence) promoter, and may include other regulatory sequences therefor. The regulatory sequences necessary are operably linked to the hGAA780I fusion protein coding sequence in a manner which permits its transcription, translation and/or expression in a target cell.

In certain embodiments, the expression cassette may include one or more miRNA target sequences in the untranslated region(s). The miRNA target sequences are designed to be specifically recognized by miRNA present in cells in which transgene expression is undesirable and/or reduced levels of transgene expression are desired. In certain embodiments, the expression cassette includes miRNA target sequences that specifically reduce expression of the hGAA780I fusion protein in dorsal root ganglion. In certain embodiments, the miRNA target sequences are located in the 3′ UTR, 5′ UTR, and/or in both 3′ and 5′ UTR. In certain embodiments, the expression cassette comprises at least two tandem repeats of dorsal root ganglion (DRG)-specific miRNA target sequences, wherein the at least two tandem repeats comprise at least a first miRNA target sequence and at least a second miRNA target sequence which may be the same or different. In certain embodiments, the start of the first of the at least two drg-specific miRNA tandem repeats is within 20 nucleotides from the 3′ end of the hGAA780I fusion protein-coding sequence. In certain embodiments, the start of the first of the at least two DRG-specific miRNA tandem repeats is at least 100 nucleotides from the 3′ end of the hGAA780I fusion protein coding sequence. In certain embodiments, the miRNA tandem repeats comprise 200 to 1200 nucleotides in length. In certain embodiment, the inclusion of miR targets does not modify the expression or efficacy of the therapeutic transgene in one or more target tissues, relative to the expression cassette or vector genome lacking the miR target sequences.

In certain embodiments, the vector genome or expression cassette contains at least one miRNA target sequence that is a miR-183 target sequence. In certain embodiments, the vector genome or expression cassette contains a miR-183 target sequence that includes AGTGAATTCTACCAGTGCCATA (SEQ ID NO: 26), where the sequence complementary to the miR-183 seed sequence is underlined. In certain embodiments, the vector genome or expression cassette contains more than one copy (e.g. two or three copies) of a sequence that is 100% complementary to the miR-183 seed sequence. In certain embodiments, a miR-183 target sequence is about 7 nucleotides to about 28 nucleotides in length and includes at least one region that is at least 100% complementary to the miR-183 seed sequence. In certain embodiments, a miR-183 target sequence contains a sequence with partial complementarity to SEQ ID NO: 26 and, thus, when aligned to SEQ ID NO: 26, there are one or more mismatches. In certain embodiments, a miR-183 target sequence comprises a sequence having at least 1, 2, 3, 4, 5, 6, 7, 8, 9, or 10 mismatches when aligned to SEQ ID NO: 26, where the mismatches may be non-contiguous. In certain embodiments, a miR-183 target sequence includes a region of 100% complementarity which also comprises at least 30% of the length of the miR-183 target sequence. In certain embodiments, the region of 100% complementarity includes a sequence with 100% complementarity to the miR-183 seed sequence. In certain embodiments, the remainder of a miR-183 target sequence has at least about 80% to about 99% complementarity to miR-183. In certain embodiments, the expression cassette or vector genome includes a miR-183 target sequence that comprises a truncated SEQ ID NO: 26, i.e., a sequence that lacks at least 1, 2, 3, 4, 5, 6, 7, 8, 9, or 10 nucleotides at either or both the 5′ or 3′ ends of SEQ ID NO: 26. In certain embodiments, the expression cassette or vector genome comprises a transgene and one miR-183 target sequence. In yet other embodiments, the expression cassette or vector genome comprises at least two, three or four miR-183 target sequences.

In certain embodiments, the vector genome or expression cassette contains at least one miRNA target sequence that is a miR-182 target sequence. In certain embodiments, the vector genome or expression cassette contains an miR-182 target sequence that includes AGTGTGAGTTCTACCATTGCCAAA (SEQ ID NO: 27). In certain embodiments, the vector genome or expression cassette contains more than one copy (e.g. two or three copies) of a sequence that is 100% complementary to the miR-182 seed sequence. In certain embodiments, a miR-182 target sequence is about 7 nucleotides to about 28 nucleotides in length and includes at least one region that is at least 100% complementary to the miR-182 seed sequence. In certain embodiments, a miR-182 target sequence contains a sequence with partial complementarity to SEQ ID NO: 27 and, thus, when aligned to SEQ ID NO: 27, there are one or more mismatches. In certain embodiments, a miR-183 target sequence comprises a sequence having at least 1, 2, 3, 4, 5, 6, 7, 8, 9, or 10 mismatches when aligned to SEQ ID NO: 27, where the mismatches may be non-contiguous. In certain embodiments, a miR-182 target sequence includes a region of 100% complementarity which also comprises at least 30% of the length of the miR-182 target sequence. In certain embodiments, the region of 100% complementarity includes a sequence with 100% complementarity to the miR-182 seed sequence. In certain embodiments, the remainder of a miR-182 target sequence has at least about 80% to about 99% complementarity to miR-182. In certain embodiments, the expression cassette or vector genome includes a miR-182 target sequence that comprises a truncated SEQ ID NO: 27, i.e., a sequence that lacks at least 1, 2, 3, 4, 5, 6, 7, 8, 9, or 10 nucleotides at either or both the 5′ or 3′ ends of SEQ ID NO: 27. In certain embodiments, the expression cassette or vector genome comprises a transgene and one miR-182 target sequence. In yet other embodiments, the expression cassette or vector genome comprises at least two, three or four miR-182 target sequences.

The term “tandem repeats” is used herein to refer to the presence of two or more consecutive miRNA target sequences. These miRNA target sequences may be continuous, i.e., located directly after one another such that the 3′ end of one is directly upstream of the 5′ end of the next with no intervening sequences, or vice versa. In another embodiment, two or more of the miRNA target sequences are separated by a short spacer sequence.

As used herein, as “spacer” is any selected nucleic acid sequence, e.g., of 1, 2, 3, 4, 5, 6, 7, 8, 9 or 10 nucleotides in length which is located between two or more consecutive miRNA target sequences. In certain embodiments, the spacer is 1 to 8 nucleotides in length, 2 to 7 nucleotides in length, 3 to 6 nucleotides in length, four nucleotides in length, 4 to 9 nucleotides, 3 to 7 nucleotides, or values which are longer. Suitably, a spacer is a non-coding sequence. In certain embodiments, the spacer may be of four (4) nucleotides. In certain embodiments, the spacer is GGAT. In certain embodiments, the spacer is six (6) nucleotides. In certain embodiments, the spacer is CACGTG or GCATGC.

In certain embodiments, the tandem repeats contain two, three, four or more of the same miRNA target sequence. In certain embodiments, the tandem repeats contain at least two different miRNA target sequences, at least three different miRNA target sequences, or at least four different miRNA target sequences, etc. In certain embodiments, the tandem repeats may contain two or three of the same miRNA target sequence and a fourth miRNA target sequence which is different.

In certain embodiments, there may be at least two different sets of tandem repeats in the expression cassette. For example, a 3′ UTR may contain a tandem repeat immediately downstream of the transgene, UTR sequences, and two or more tandem repeats closer to the 3′ end of the UTR. In another example, the 5′ UTR may contain one, two or more miRNA target sequences. In another example the 3′ may contain tandem repeats and the 5′ UTR may contain at least one miRNA target sequence.

In certain embodiments, the expression cassette contains two, three, four or more tandem repeats which start within about 0 to 20 nucleotides of the stop codon for the transgene. In other embodiments, the expression cassette contains the miRNA tandem repeats at least 100 to about 4000 nucleotides from the stop codon for the transgene.

See, PCT/US19/67872, filed Dec. 20, 2019, which is incorporated by reference herein and which claims priority to US Provisional U.S. Patent Application No. 62/783,956, filed Dec. 21, 2018, which is hereby incorporated by reference.

As used herein, “BiP-vIGF2.hGAAcoV780I.4xmir183” refers to an expression cassette (e.g., as depicted in FIG. 11) that contains a engineered coding sequence for a hGAA780I having a modified BiP-vIGF2 signal sequence under the control of the ubiquitous CAG promoter, and four tandem repeats of miR183 target sequences. As illustrated in the Examples provided herein, both the V780I mutation and the BiP-vIGF2 modifications contribute to improved safety and efficacy. In certain embodiments, the BiP-vIGF2.hGAAcoV780I.4xmir183 includes a sequence encoding a fusion protein of SEQ ID NO: 3, or a sequence at least 95% identical thereto. In certain embodiments, the BiP-vIGF2.hGAAcoV780I.4xmir183 includes the nucleic acid sequence of SEQ ID NO: 7, or a sequence at least 95% to 99% identical thereto. In yet another embodiment, provided herein is a vector genome, wherein BiP-vIGF2.hGAAcoV780I.4xmir183 is flanked by a 5′ ITR and a 3′ ITR. In certain embodiments the vector genome is SEQ ID NO: 30. In yet a further embodiment, a vector genome is provided that included a sequence at least 95% identical to SEQ ID NO: 30 and encodes the fusion protein of SEQ ID NO: 6.

As used herein, “operably linked” sequences include both expression control sequences that are contiguous with the hGAA780I coding sequence and expression control sequences that act in trans or at a distance to control the hGAA780I coding sequence. Such regulatory sequences typically include, e.g., one or more of a promoter, an enhancer, an intron, a Kozak sequence, a polyadenylation sequence, and a TATA signal.

In certain embodiments, the regulatory elements direct expression in multiple cells and tissues affected by Pompe disease, in order to permit construction and delivery of a single expression cassette suitable for treating multiple target cells. For examples, regulatory elements (e.g., a promoter) may be selected which express in two or more of liver, skeletal muscle, heart and central nervous system cells. For example, regulatory elements (e.g., a promoter) may be selected which expresses in central nervous system (e.g., brain) cells, and skeletal muscle). In other embodiments, the regulatory elements express in CNS, skeletal muscle and heart. In other embodiments, the expression cassette permits expression of an encoded hGAA780I in all of liver, skeletal muscle, heart and central nervous system cells. In other embodiments, regulatory elements may be selected for targeting specific tissue and avoiding expression in certain cells or tissue (e.g., by use of the drg-detargeting system described herein and/or by selection of a tissue-specific promoter). In certain embodiments, different expression cassettes provided herein are administered to a patient which preferentially target different tissues.

The regulatory sequences comprise a promoter. Suitable promoters may be selected, including but not limited to a promoter which will express an hGAAV780I protein in the targeted cells.

In certain embodiments, a constitutive promoter or an inducible/regulatory promoter is selected. An example of a constitutive promoter is chicken beta-actin promoter. A variety of chicken beta-actin promoters have been described alone, or in combination with various enhancer elements (e.g., CB7 is a chicken beta-actin promoter with cytomegalovirus enhancer elements; a CAG promoter, which includes the promoter, the first exon and first intron of chicken beta actin, and the splice acceptor of the rabbit beta-globin gene; a CBh promoter, S J Gray et al, Hu Gene Ther, 2011 September; 22(9): 1143-1153). In certain embodiments, a regulatable promoter may be selected. See, e.g., WO 2011/126808B2, which is incorporated by reference herein.

In certain embodiments, a tissue-specific promoter may be selected. Examples of promoters that are tissue-specific are well known for liver (albumin, Miyatake et al., (1997) J. Virol., 71:5124-32; hepatitis B virus core promoter, Sandig et al., (1996) Gene Ther., 3:1002-9; alpha-fetoprotein (AFP), Arbuthnot et al., (1996) Hum. Gene Ther., 7:1503-14), central nervous system, e.g., neuron (such as neuron-specific enolase (NSE) promoter, Andersen et al., (1993) Cell. Mol. Neurobiol., 13:503-15; neurofilament light-chain gene, Piccioli et al., (1991) Proc. Natl. Acad. Sci. USA, 88:5611-5; and the neuron-specific vgf gene, Piccioli et al., (1995) Neuron, 15:373-84), cardiac muscle, skeletal muscle, lung, and other tissues. In another embodiment, a suitable promoter may include without limitation, an elongation factor 1 alpha (EF1 alpha) promoter (see, e.g., Kim D W et al, Use of the human elongation factor 1 alpha promoter as a versatile and efficient expression system. Gene. 1990 Jul. 16; 91(2):217-23), a Synapsin 1 promoter (see, e.g., Kugler S et al, Human synapsin 1 gene promoter confers highly neuron-specific long-term transgene expression from an adenoviral vector in the adult rat brain depending on the transduced area. Gene Ther. 2003 February; 10(4):337-47), a neuron-specific enolase (NSE) promoter (see, e.g., Kim J et al, Involvement of cholesterol-rich lipid rafts in interleukin-6-induced neuroendocrine differentiation of LNCaP prostate cancer cells. Endocrinology. 2004 February; 145(2):613-9. Epub 2003 Oct. 16), or a CB6 promoter (see, e.g., Large-Scale Production of Adeno-Associated Viral Vector Serotype-9 Carrying the Human Survival Motor Neuron Gene, Mol Biotechnol. 2016 January; 58(1):30-6. doi: 10.1007/s12033-015-9899-5). In certain embodiments utilizing tissue-specific promoters, co-therapies may be selected which involve different expression cassettes with tissue-specific promoters which target different cell types.

In one embodiment, the regulatory sequence further comprises an enhancer. In one embodiment, the regulatory sequence comprises one enhancer. In another embodiment, the regulatory sequence contains two or more expression enhancers. These enhancers may be the same or may be different. For example, an enhancer may include an Alpha mic/bik enhancer or a CMV enhancer. This enhancer may be present in two copies which are located adjacent to one another. Alternatively, the dual copies of the enhancer may be separated by one or more sequences.

In one embodiment, the regulatory sequence further comprises an intron. In a further embodiment, the intron is a chicken beta-actin intron. Other suitable introns include those known in the art may by a human β-globulin intron, and/or a commercially available Promega® intron, and those described in WO 2011/126808.

In one embodiment, the regulatory sequence further comprises a Polyadenylation signal (polyA). In a further embodiment, the polyA is a rabbit globin poly A. See, e.g., WO 2014/151341. Alternatively, another polyA, e.g., a human growth hormone (hGH) polyadenylation sequence, an SV40 polyA, or a synthetic polyA may be included in an expression cassette.

It should be understood that the compositions in the expression cassette described herein are intended to be applied to other compositions, regimens, aspects, embodiments and methods described across the Specification.

Expression cassettes can be delivered via any suitable delivery system. Suitable non-viral delivery systems are known in the art (see, e.g., Ramamoorth and Narvekar. J Clin Diagn Res. 2015 January; 9(1):GE01-GE06, which is incorporated herein by reference) and can be readily selected by one of skill in the art and may include, e.g., naked DNA, naked RNA, dendrimers, PLGA, polymethacrylate, an inorganic particle, a lipid particle (e.g., a lipid nanoparticle or LNP), or a chitosan-based formulation.

In one embodiment, the vector is a non-viral plasmid that comprises an expression cassette described thereof, e.g., “naked DNA”, “naked plasmid DNA”, RNA, and mRNA; coupled with various compositions and nano particles, including, e.g., micelles, liposomes, cationic lipid-nucleic acid compositions, poly-glycan compositions and other polymers, lipid and/or cholesterol-based-nucleic acid conjugates, and other constructs such as are described herein. See, e.g., X. Su et al, Mol. Pharmaceutics, 2011, 8 (3), pp 774-787; web publication: Mar. 21, 2011; WO2013/182683, WO 2010/053572 and WO 2012/170930, all of which are incorporated herein by reference.

In certain embodiments, provided herein are nucleic acid molecules having sequences encoding a hGAA780I variant, a fusion protein, or a truncated protein, as described herein. In one desirable embodiment, the hGAA780I is encoded by the engineered sequence of SEQ ID NO: 4 or a sequence at least 95% identical thereto which encodes the hGAA780I variant. In certain embodiments, SEQ ID NO: 4 is modified such that the codon encoding the Ile at position 780I is ATT or ATC. In certain embodiments, a nucleic acid comprising the engineered sequence of SEQ ID NO: 4, or a fragment thereof, is used to express a fusion protein or truncated hGAA780I. Although less desirable, in certain embodiments, the hGAA780I is encoded by SEQ ID NO: 5. In certain embodiments, the nucleic acid encodes a fusion protein having the amino acid sequence of SEQ ID NO: 6, or a sequence at least 95% identical thereto. In certain embodiments, a nucleic acid is provided having the sequence of SEQ ID NO: 7, or a sequence at least 95% identical thereto. In certain embodiments, the nucleic acid molecule is a plasmid.

### Vectors

A “vector” as used herein is a biological or chemical moiety comprising a nucleic acid sequence which can be introduced into an appropriate target cell for replication or expression of the nucleic acid sequence. Examples of a vector include but are not limited to a recombinant virus, a plasmid, Lipoplexes, a Polymersome, Polyplexes, a dendrimer, a cell penetrating peptide (CPP) conjugate, a magnetic particle, or a nanoparticle. In one embodiment, a vector is a nucleic acid molecule having an exogenous or heterologous engineered nucleic acid encoding a functional gene product, which can then be introduced into an appropriate target cell. Such vectors preferably have one or more origins of replication, and one or more site into which the recombinant DNA can be inserted. Vectors often have means by which cells with vectors can be selected from those without, e.g., they encode drug resistance genes. Common vectors include plasmids, viral genomes, and “artificial chromosomes”. Conventional methods of generation, production, characterization, or quantification of the vectors are available to one of skill in the art.

In certain embodiments, the vector described herein is a “replication-defective virus” or a “viral vector” which refers to a synthetic or artificial viral particle in which an expression cassette containing a nucleic acid sequence encoding a functional hGAA780I fusion protein packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”-containing only the nucleic acid sequence encoding flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

As used herein, a recombinant viral vector is any suitable viral vector which targets the desired cell(s). Thus, a recombinant viral vector preferably targets one or more of the cells and tissues affect affected by Pompe disease, including, central nervous system (e.g., brain), skeletal muscle, heart, and/or liver. In certain embodiments, the viral vector targets at least the central nervous system (e.g., brain) cells, lung, cardiac cells, or skeletal muscle. In other embodiments, the viral vector targets CNS (e.g., brain), skeletal muscle and/or heart. In other embodiments, the viral vector targets all of liver, skeletal muscle, heart and central nervous system cells. The examples provide illustrative recombinant adeno-associated viruses (rAAV). However, other suitable viral vectors may include, e.g., a recombinant adenovirus, a recombinant parvovirus such a recombinant bocavirus, a hybrid AAV/bocavirus, a recombinant herpes simplex virus, a recombinant retrovirus, or a recombinant lentivirus. In preferred embodiments, these recombinant viruses are replication-incompetent.

As used herein, the term “host cell” may refer to the packaging cell line in which a vector (e.g., a recombinant AAV) is produced. A host cell may be a prokaryotic or eukaryotic cell (e.g., human, insect, or yeast) that contains exogenous or heterologous DNA that has been introduced into the cell by any means, e.g., electroporation, calcium phosphate precipitation, microinjection, transformation, viral infection, transfection, liposome delivery, membrane fusion techniques, high velocity DNA-coated pellets, viral infection and protoplast fusion. Examples of host cells may include, but are not limited to an isolated cell, a cell culture, an Escherichia coli cell, a yeast cell, a human cell, a non-human cell, a mammalian cell, a non-mammalian cell, an insect cell, an HEK-293 cell, a liver cell, a kidney cell, a cell of the central nervous system, a neuron, a glial cell, or a stem cell.

In certain embodiments, a host cell contains an expression cassette for production of hGAA780I such that the protein is produced in sufficient quantities in vitro for isolation or purification. In certain embodiments, the host cell contains an expression cassette encoding hGAAV780I, or a fragment thereof. As provided herein, hGAA780I may be included in a pharmaceutical composition administered to a subject as a therapeutic (i. e, enzyme replacement therapy).

As used herein, the term “target cell” refers to any target cell in which expression of the functional gene product is desired.

As used herein, a “vector genome” refers to the nucleic acid sequence packaged inside a viral vector. In one example, a “vector genome” contains, at a minimum, from 5′ to 3′, a vector-specific sequence, a nucleic acid sequence encoding a functional gene product (e.g., a hGAAV780I, a fusion protein hGAAV780I, or another protein) operably linked to regulatory control sequences which direct it expression in a target cell, a vector-specific sequence, and optionally, miRNA target sequences in the untranslated region(s) and a vector-specific sequence. A vector-specific sequence may be a terminal repeat sequence which specifically packages of the vector genome into a viral vector capsid or envelope protein. For example, AAV inverted terminal repeats are utilized for packaging into AAV and certain other parvovirus capsids. Lentivirus long terminal repeats may be utilized where packaging into a lentiviral vector is desired. Similarly, other terminal repeats (e.g., a retroviral long terminal repeat), or the like may be selected.

It should be understood that the compositions in the vector described herein are intended to be applied to other compositions, regimens, aspects, embodiments, and methods described across the Specification.

### Adeno-Associated Virus (AAV)

In one aspect, provided herein is a recombinant AAV (rAAV) comprising an AAV capsid and a vector genome packaged therein which encodes an hGAAV780I fusion protein (enzyme) as described herein. In certain embodiments, the AAV capsid selected targets cells of two or more of liver, muscle, kidney, heart and/or a central nervous system cell type. In certain embodiments, it is desirable to express the hGAA780I fusion protein in at least two or more of liver, skeletal muscle, heart, kidney and/or at least one central nervous system cell type. Thus, in one embodiment the AAV capsid selected targets cardiac tissue. In certain embodiments, the AAV capsid selected to target cardiac tissue is selected from AAV 1, 6, 8, and 9 (see, e.g. Katz et al. Hum Gene Ther Clin Dev. 2017 Sep. 1; 28(3): 157-164). In yet other embodiments, the AAV capsid selected target cells of the kidney. In one embodiment, a capsid for targeting kidney cells is selected from AAV1, 2, 6, 8, 9, and Anc80 (see, e.g., Ikeda Y et al. J Am Soc Nephrol. 2018 September; 29(9):2287-2297 and Ascio et al. Biochem Biophys Res Commun. 2018 Feb. 26; 497(1): 19-24). In certain embodiments, the AAV capsid is a natural or engineered clade F capsid. In certain embodiments, the capsid is an AAV9 capsid or an AAVhu68 capsid.

In one embodiment, the vector genome comprises an AAV 5′ inverted terminal repeat (ITR), an expression cassette as described herein, and an AAV 3′ ITR. In one embodiment, the vector genome refers to the nucleic acid sequence packaged inside a rAAV capsid forming an rAAV vector. Such a nucleic acid sequence contains AAV inverted terminal repeat sequences (ITRs) flanking an expression cassette. In one example, a “vector genome” for packaging into an AAV or bocavirus capsid contains, at a minimum, from 5′ to 3′, an AAV 5′ ITR, a nucleic acid sequence encoding a functional hGAA780I fusion protein as described herein operably linked to regulatory control sequences which direct it expression in a target cell and an AAV 3′ ITR. In certain embodiments, the ITRs are from AAV2 and the capsid is from a different AAV. Alternatively, other ITRs may be used. In certain embodiments, the vector genome further comprises miRNA target sequences in the untranslated region(s) which are designed to be specifically recognized by miRNA sequences in cells in which transgene expression is undesirable and/or reduced levels of transgene expression are desired.

The ITRs are the genetic elements responsible for the replication and packaging of the genome during vector production and are the only viral cis elements required to generate rAAV. In one embodiment, the ITRs are from an AAV different than that supplying a capsid. In a preferred embodiment, the ITR sequences from AAV2, or the deleted version thereof (ΔITR), which may be used for convenience and to accelerate regulatory approval. However, ITRs from other AAV sources may be selected. Where the source of the ITRs is from AAV2 and the AAV capsid is from another AAV source, the resulting vector may be termed pseudotyped. Typically, AAV vector genome comprises an AAV 5′ ITR, the hGAA780I coding sequence and any regulatory sequences, and an AAV 3′ ITR. However, other configurations of these elements may be suitable. A shortened version of the 5′ ITR, termed ΔITR, has been described in which the D-sequence and terminal resolution site (trs) are deleted. In other embodiments, the full-length AAV 5′ and 3′ ITRs are used.

The term “AAV” as used herein refers to naturally occurring adeno-associated viruses, adeno-associated viruses available to one of skill in the art and/or in light of the composition(s) and method(s) described herein, as well as artificial AAVs. An adeno-associated virus (AAV) viral vector is an AAV nuclease (e.g., DNase)-resistant particle having an AAV protein capsid into which is packaged expression cassette flanked by AAV inverted terminal repeat sequences (ITRs) for delivery to target cells. A nuclease-resistant recombinant AAV (rAAV) indicates that the AAV capsid has fully assembled and protects these packaged vector genome sequences from degradation (digestion) during nuclease incubation steps designed to remove contaminating nucleic acids which may be present from the production process. In many instances, the rAAV described herein is DNase resistant.

An AAV capsid is composed of 60 capsid (cap) protein subunits, VP1, VP2, and VP3, that are arranged in an icosahedral symmetry in a ratio of approximately 1:1:10 to 1:1:20, depending upon the selected AAV. Various AAVs may be selected as sources for capsids of AAV viral vectors as identified above. See, e.g., US Published Patent Application No. 2007-0036760-A1; US Published Patent Application No. 2009-0197338-A1; EP 1310571. See also, WO 2003/042397 (AAV7 and other simian AAV), U.S. Pat. Nos. 7,790,449 and 7,282,199 (AAV8), WO 2005/033321 and U.S. Pat. No. 7,906,111 (AAV9), and WO 2006/110689, and WO 2003/042397 (rh.10). These documents also describe other AAV which may be selected for generating AAV and are incorporated by reference. Among the AAVs isolated or engineered from human or non-human primates (NHP) and well characterized, human AAV2 is the first AAV that was developed as a gene transfer vector; it has been widely used for efficient gene transfer experiments in different target tissues and animal models. Unless otherwise specified, the AAV capsid, ITRs, and other selected AAV components described herein, may be readily selected from among any AAV, including, without limitation, the AAVs commonly identified as AAV1, AAV2, AAV3, AAV4, AAV5, AAV6, AAV7, AAV8, AAV9, AAV8 bp, AAV7M8 and AAVAnc80. See, e.g., WO 2005/033321, which is incorporated herein by reference. In one embodiment, the AAV capsid is an AAV9 capsid or variant thereof. In certain embodiments, the capsid protein is designated by a number or a combination of numbers and letters following the term “AAV” in the name of the rAAV vector.

The ITRs or other AAV components may be readily isolated or engineered using techniques available to those of skill in the art from an AAV. Such AAV may be isolated, engineered, or obtained from academic, commercial, or public sources (e.g., the American Type Culture Collection, Manassas, Va.). Alternatively, the AAV sequences may be engineered through synthetic or other suitable means by reference to published sequences such as are available in the literature or in databases such as, e.g., GenBank, PubMed, or the like. AAV viruses may be engineered by conventional molecular biology techniques, making it possible to optimize these particles for cell specific delivery of nucleic acid sequences, for minimizing immunogenicity, for tuning stability and particle lifetime, for efficient degradation, for accurate delivery to the nucleus, etc.

As used herein, the terms “rAAV” and “artificial AAV” used interchangeably, mean, without limitation, a AAV comprising a capsid protein and a vector genome packaged therein, wherein the vector genome comprising a nucleic acid heterologous to the AAV. In one embodiment, the capsid protein is a non-naturally occurring capsid. Such an artificial capsid may be generated by any suitable technique, using a selected AAV sequence (e.g., a fragment of a vp1 capsid protein) in combination with heterologous sequences which may be obtained from a different selected AAV, non-contiguous portions of the same AAV, from a non-AAV viral source, or from a non-viral source. An artificial AAV may be, without limitation, a pseudotyped AAV, a chimeric AAV capsid, a recombinant AAV capsid, or a “humanized” AAV capsid. Pseudotyped vectors, wherein the capsid of one AAV is replaced with a heterologous capsid protein, are useful in certain embodiments. In one embodiment, AAV2/5 and AAV2/8 are exemplary pseudotyped vectors. The selected genetic element may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, N.Y. (2012).

In certain embodiments, the AAV capsid is selected from among natural and engineered clade F adeno-associated viruses. In the examples below, the clade F adeno-associated virus is AAVhu68. See, WO 2018/160582, which is incorporated by reference herein in its entirety. However, in other embodiments, an AAV capsid is selected from a different clade, e.g., clade A, B, C, D, or E, or from an AAV source outside of any of these clades.

As used herein, the term “clade” as it relates to groups of AAV refers to a group of AAV which are phylogenetically related to one another as determined using a Neighbor-Joining algorithm by a bootstrap value of at least 75% (of at least 1000 replicates) and a Poisson correction distance measurement of no more than 0.05, based on alignment of the AAV vp1 amino acid sequence. The Neighbor-Joining algorithm has been described in the literature. See, e.g., M. Nei and S. Kumar, Molecular Evolution and Phylogenetics (Oxford University Press, New York (2000). Computer programs are available that can be used to implement this algorithm. For example, the MEGA v2.1 program implements the modified Nei-Gojobori method. Using these techniques and computer programs, and the sequence of an AAV vp1 capsid protein, one of skill in the art can readily determine whether a selected AAV is contained in one of the clades identified herein, in another clade, or is outside these clades. See, e.g., G Gao, et al, J Virol, 2004 June; 7810: 6381-6388, which identifies Clades A, B, C, D, E and F, and provides nucleic acid sequences of novel AAV, GenBank Accession Numbers AY530553 to AY530629. See, also, WO 2005/033321.

As used herein, “AAV9 capsid” refers to the AAV9 having the amino acid sequence of (a) GenBank accession: AAS99264, is incorporated by reference herein and the AAV vp1 capsid protein and/or (b) the amino acid sequence encoded by the nucleotide sequence of GenBank Accession: AY530579.1: (nt 1 . . . 2211). Some variation from this encoded sequence is encompassed by the present invention, which may include sequences having about 99% identity to the referenced amino acid sequence in GenBank accession: AAS99264 and U.S. Pat. No. 7,906,111 (also WO 2005/033321) (i.e., less than about 1% variation from the referenced sequence). Such AAV may include, e.g., natural isolates (e.g., hu31 or hu32), or variants of AAV9 having amino acid substitutions, deletions or additions, e.g., including but not limited to amino acid substitutions selected from alternate residues “recruited” from the corresponding position in any other AAV capsid aligned with the AAV9 capsid; e.g., such as described in U.S. Pat. Nos. 9,102,949, 8,927,514, US2015/349911, WO 2016/049230A1, U.S. Pat. Nos. 9,623,120, and 9,585,971. However, in other embodiments, other variants of AAV9, or AAV9 capsids having at least about 95% identity to the above-referenced sequences may be selected. See, e.g., US 2015/0079038. Methods of generating the capsid, coding sequences therefore, and methods for production of rAAV viral vectors have been described. See, e.g., Gao, et al, Proc. Natl. Acad. Sci. U.S.A. 100 (10), 6081-6086 (2003) and US 2013/0045186A1.

In certain embodiments, an AAVhu68 capsid is as described in WO 2018/160582, entitled “Novel Adeno-associated virus (AAV) Clade F Vector and Uses Therefor”, which is hereby incorporated by reference. In certain embodiments, AAVhu68 capsid proteins comprise: AAVhu68 vp1 proteins produced by expression from a nucleic acid sequence which encodes the predicted amino acid sequence of 1 to 736 of SEQ ID NO: 2, vp1 proteins produced from SEQ ID NO: 2 or vp1 proteins produced from a nucleic acid sequence at least 70% identical to SEQ ID NO: 1 which encodes the predicted amino acid sequence of 1 to 736 of SEQ ID NO: 2; AAVhu68 vp2 proteins produced by expression from a nucleic acid sequence which encodes the predicted amino acid sequence of at least about amino acids 138 to 736 of SEQ ID NO: 2, vp2 proteins produced from a sequence comprising at least nucleotides 412 to 2211 of SEQ ID NO: 1, or vp2 proteins produced from a nucleic acid sequence at least 70% identical to at least nucleotides 412 to 2211 of SEQ ID NO:1 which encodes the predicted amino acid sequence of at least about amino acids 138 to 736 of SEQ ID NO: 2, and/or AAVhu68 vp3 proteins produced by expression from a nucleic acid sequence which encodes the predicted amino acid sequence of at least about amino acids 203 to 736 of SEQ ID NO: 2, vp3 proteins produced from a sequence comprising at least nucleotides 607 to 2211 of SEQ ID NO: 1, or vp3 proteins produced from a nucleic acid sequence at least 70% identical to at least nucleotides 607 to 2211 of SEQ ID NO: 1 which encodes the predicted amino acid sequence of at least about amino acids 203 to 736 of SEQ ID NO: 2.

The AAVhu68 vp1, vp2 and vp3 proteins are typically expressed as alternative splice variants encoded by the same nucleic acid sequence which encodes the full-length vp1 amino acid sequence of SEQ ID NO: 2 (amino acid 1 to 736). Optionally the vp1-encoding sequence is used alone to express the vp1, vp2, and vp3 proteins. Alternatively, this sequence may be co-expressed with one or more of a nucleic acid sequence which encodes the AAVhu68 vp3 amino acid sequence of SEQ ID NO: 2 (about aa 203 to 736) without the vp1-unique region (about aa 1 to about aa 137) and/or vp2-unique regions (about aa 1 to about aa 202), or a strand complementary thereto, the corresponding mRNA (about nt 607 to about nt 2211 of SEQ ID NO: 1), or a sequence at least 70% to at least 99% (e.g., at least 85%, at least 90%, at least 95%, at least 97%, at least 98% or at least 99%) identical to SEQ ID NO: 1 which encodes aa 203 to 736 of SEQ ID NO: 2. Additionally, or alternatively, the vp1-encoding and/or the vp2-encoding sequence may be co-expressed with the nucleic acid sequence which encodes the AAVhu68 vp2 amino acid sequence of SEQ ID NO: 2 (about aa 138 to 736) without the vp1-unique region (about aa 1 to about 137), or a strand complementary thereto, the corresponding mRNA (nt 412 to 2211 of SEQ ID NO: 1), or a sequence at least 70% to at least 99% (e.g., at least 85%, at least 90%, at least 95%, at least 97%, at least 98% or at least 99%) identical to nt 412 to 2211 of SEQ ID NO: 1 which encodes about aa 138 to 736 of SEQ ID NO: 2.

As described herein, a rAAVhu68 has a rAAVhu68 capsid produced in a production system expressing capsids from an AAVhu68 nucleic acid which encodes the vp1 amino acid sequence of SEQ ID NO: 2, and optionally additional nucleic acid sequences, e.g., encoding a vp3 protein free of the vp1 and/or vp2-unique regions. The rAAVhu68 resulting from production using a single nucleic acid sequence vp1 produces the heterogenous populations of vp1 proteins, vp2 proteins and vp3 proteins. More particularly, the AAVhu68 capsid contains subpopulations within the vp1 proteins, within the vp2 proteins and within the vp3 proteins which have modifications from the predicted amino acid residues in SEQ ID NO: 2. These subpopulations include, at a minimum, deamidated asparagine (N or Asn) residues. For example, asparagines in asparagine-glycine pairs are highly deamidated.

In one embodiment, the AAVhu68 vp1 nucleic acid sequence has the sequence of SEQ ID NO: 1, or a strand complementary thereto, e.g., the corresponding mRNA. In certain embodiments, the vp2 and/or vp3 proteins may be expressed additionally or alternatively from different nucleic acid sequences than the vp1, e.g., to alter the ratio of the vp proteins in a selected expression system. In certain embodiments, also provided is a nucleic acid sequence which encodes the AAVhu68 vp3 amino acid sequence of SEQ ID NO: 2 (about aa 203 to 736) without the vp1-unique region (about aa 1 to about aa 137) and/or vp2-unique regions (about aa 1 to about aa 202), or a strand complementary thereto, the corresponding mRNA (about nt 607 to about nt 2211 of SEQ ID NO: 2). In certain embodiments, also provided is a nucleic acid sequence which encodes the AAVhu68 vp2 amino acid sequence of SEQ ID NO: 2 (about aa 138 to 736) without the vp1-unique region (about aa 1 to about 137), or a strand complementary thereto, the corresponding mRNA (nt 412 to 2211 of SEQ ID NO: 1).

However, other nucleic acid sequences which encode the amino acid sequence of SEQ ID NO: 2 may be selected for use in producing rAAVhu68 capsids. In certain embodiments, the nucleic acid sequence has the nucleic acid sequence of SEQ ID NO: 1 or a sequence at least 70% to 99% identical, at least 75%, at least 80%, at least 85%, at least 90%, at least 95%, at least 97%, at least 99%, identical to SEQ ID NO: 1 which encodes SEQ ID NO: 2. In certain embodiments, the nucleic acid sequence has the nucleic acid sequence of SEQ ID NO: 1 or a sequence at least 70% to 99%, at least 75%, at least 80%, at least 85%, at least 90%, at least 95%, at least 97%, or at least 99% identical to about nt 412 to about nt 2211 of SEQ ID NO: 1 which encodes the vp2 capsid protein (about aa 138 to 736) of SEQ ID NO: 2. In certain embodiments, the nucleic acid sequence has the nucleic acid sequence of about nt 607 to about nt 2211 of SEQ ID NO:1 or a sequence at least 70% to 99.%, at least 75%, at least 80%, at least 85%, at least 90%, at least 95%, at least 97%, or at least 99% identical to nt 412 to about nt 2211 of SEQ ID NO: 1 which encodes the vp3 capsid protein (about aa 203 to 736) of SEQ ID NO: 1.

It is within the skill in the art to design nucleic acid sequences encoding this AAVhu68 capsid, including DNA (genomic or cDNA), or RNA (e.g., mRNA). In certain embodiments, the nucleic acid sequence encoding the AAVhu68 vp1 capsid protein is provided in SEQ ID NO: 2. In certain embodiments, the AAVhu68 capsid is produced using a nucleic acid sequence of SEQ ID NO: 1 or a sequence at least 70%, at least 75%, at least 80%, at least 85%, at least 90%, at least 95%, at least 97%, or at least 99% which encodes the vp1 amino acid sequence of SEQ ID NO: 2 with a modification (e.g., deamidated amino acid) as described herein. In certain embodiments, the vp1 amino acid sequence is reproduced in SEQ ID NO: 2.

In certain embodiments, AAV capsids having reduced capsid deamidation may be selected. See, e.g., PCT/US19/19804 and PCT/US18/19861, both filed Feb. 27, 2019 and incorporated by reference in their entireties.

As used herein when used to refer to vp capsid proteins, the term “heterogenous” or any grammatical variation thereof, refers to a population consisting of elements that are not the same, for example, having vp1, vp2 or vp3 monomers (proteins) with different modified amino acid sequences. SEQ ID NO: 2 provides the encoded amino acid sequence of the AAVhu68 vp1 protein. The term “heterogenous” as used in connection with vp1, vp2 and vp3 proteins (alternatively termed isoforms), refers to differences in the amino acid sequence of the vp1, vp2 and vp3 proteins within a capsid. The AAV capsid contains subpopulations within the vp1 proteins, within the vp2 proteins and within the vp3 proteins which have modifications from the predicted amino acid residues. These subpopulations include, at a minimum, certain deamidated asparagine (N or Asn) residues. For example, certain subpopulations comprise at least one, two, three or four highly deamidated asparagines (N) positions in asparagine-glycine pairs and optionally further comprising other deamidated amino acids, wherein the deamidation results in an amino acid change and other optional modifications.

As used herein, a “subpopulation” of vp proteins refers to a group of vp proteins which has at least one defined characteristic in common and which consists of at least one group member to less than all members of the reference group, unless otherwise specified. For example, a “subpopulation” of vp1 proteins is at least one (1) vp1 protein and less than all vp1 proteins in an assembled AAV capsid, unless otherwise specified. A “subpopulation” of vp3 proteins may be one (1) vp3 protein to less than all vp3 proteins in an assembled AAV capsid, unless otherwise specified. For example, vp1 proteins may be a subpopulation of vp proteins; vp2 proteins may be a separate subpopulation of vp proteins, and vp3 are yet a further subpopulation of vp proteins in an assembled AAV capsid. In another example, vp1, vp2 and vp3 proteins may contain subpopulations having different modifications, e.g., at least one, two, three or four highly deamidated asparagines, e.g., at asparagine-glycine pairs.

Unless otherwise specified, highly deamidated refers to at least 45% deamidated, at least 50% deamidated, at least 60% deamidated, at least 65% deamidated, at least 70%, at least 75%, at least 80%, at least 85%, at least 90%, at least 95%, at least 97%, at least 99%, or up to about 100% deamidated at a referenced amino acid position, as compared to the predicted amino acid sequence at the reference amino acid position (e.g., at least 80% of the asparagines at amino acid 57 based on the numbering of SEQ ID NO: 2 [AAVhu68] may be deamidated based on the total vp1 proteins may be deamidated based on the total vp1, vp2 and vp3 proteins). Such percentages may be determined using 2D-gel, mass spectrometry techniques, or other suitable techniques.

Thus, an rAAV includes subpopulations within the rAAV capsid of vp1, vp2, and/or vp3 proteins with deamidated amino acids, including at a minimum, at least one subpopulation comprising at least one highly deamidated asparagine. In addition, other modifications may include isomerization, particularly at selected aspartic acid (D or Asp) residue positions. In still other embodiments, modifications may include an amidation at an Asp position.

In certain embodiments, an AAV capsid contains subpopulations of vp1, vp2 and vp3 having at least 4 to at least about 25 deamidated amino acid residue positions, of which at least 1 to 10% are deamidated as compared to the encoded amino acid sequence of the vp proteins. The majority of these may be N residues. However, Q residues may also be deamidated.

In certain embodiments, a rAAV has an AAV capsid having vp1, vp2 and vp3 proteins having subpopulations comprising combinations of two, three, four or more deamidated residues at the positions set forth in the table provided in Example 1 and incorporated herein by reference. Deamidation in the rAAV may be determined using 2D gel electrophoresis, and/or mass spectrometry, and/or protein modelling techniques. Online chromatography may be performed with an Acclaim PepMap column and a Thermo UltiMate 3000 RSLC system (Thermo Fisher Scientific) coupled to a Q Exactive HF with a NanoFlex source (Thermo Fisher Scientific). MS data is acquired using a data-dependent top-20 method for the Q Exactive HF, dynamically choosing the most abundant not-yet-sequenced precursor ions from the survey scans (200-2000 m/z). Sequencing is performed via higher energy collisional dissociation fragmentation with a target value of 1e5 ions determined with predictive automatic gain control and an isolation of precursors was performed with a window of 4 m/z. Survey scans were acquired at a resolution of 120,000 at m/z 200. Resolution for HCD spectra may be set to 30,000 at m/z200 with a maximum ion injection time of 50 ms and a normalized collision energy of 30. The S-lens RF level may be set at 50, to give optimal transmission of the m/z region occupied by the peptides from the digest. Precursor ions may be excluded with single, unassigned, or six and higher charge states from fragmentation selection. BioPharma Finder 1.0 software (Thermo Fischer Scientific) may be used for analysis of the data acquired. For peptide mapping, searches are performed using a single-entry protein FASTA database with carbamidomethylation set as a fixed modification; and oxidation, deamidation, and phosphorylation set as variable modifications, a 10-ppm mass accuracy, a high protease specificity, and a confidence level of 0.8 for MS/MS spectra. Examples of suitable proteases may include, e.g., trypsin or chymotrypsin. Mass spectrometric identification of deamidated peptides is relatively straightforward, as deamidation adds to the mass of intact molecule+0.984 Da (the mass difference between —OH and —NH² groups). The percent deamidation of a particular peptide is determined by the mass area of the deamidated peptide divided by the sum of the area of the deamidated and native peptides. Considering the number of possible deamidation sites, isobaric species which are deamidated at different sites may co-migrate in a single peak. Consequently, fragment ions originating from peptides with multiple potential deamidation sites can be used to locate or differentiate multiple sites of deamidation. In these cases, the relative intensities within the observed isotope patterns can be used to specifically determine the relative abundance of the different deamidated peptide isomers. This method assumes that the fragmentation efficiency for all isomeric species is the same and independent on the site of deamidation. It is understood by one of skill in the art that a number of variations on these illustrative methods can be used. For example, suitable mass spectrometers may include, e.g., a quadrupole time of flight mass spectrometer (QTOF), such as a Waters Xevo or Agilent 6530 or an orbitrap instrument, such as the Orbitrap Fusion or Orbitrap Velos (Thermo Fisher). Suitably liquid chromatography systems include, e.g., Acquity UPLC system from Waters or Agilent systems (1100 or 1200 series). Suitable data analysis software may include, e.g., MassLynx (Waters), Pinpoint and Pepfinder (Thermo Fischer Scientific), Mascot (Matrix Science), Peaks DB (Bioinformatics Solutions). Still other techniques may be described, e.g., in X. Jin et al, Hu Gene Therapy Methods, Vol. 28, No. 5, pp. 255-267, published online Jun. 16, 2017.

In addition to deamidations, other modifications may occur do not result in conversion of one amino acid to a different amino acid residue. Such modifications may include acetylated residues, isomerizations, phosphorylations, or oxidations.

Modulation of Deamidation: In certain embodiments, the AAV is modified to change the glycine in an asparagine-glycine pair, to reduce deamidation. In other embodiments, the asparagine is altered to a different amino acid, e.g., a glutamine which deamidates at a slower rate; or to an amino acid which lacks amide groups (e.g., glutamine and asparagine contain amide groups); and/or to an amino acid which lacks amine groups (e.g., lysine, arginine and histidine contain amine groups). As used herein, amino acids lacking amide or amine side groups refer to, e.g., glycine, alanine, valine, leucine, isoleucine, serine, threonine, cystine, phenylalanine, tyrosine, or tryptophan, and/or proline. Modifications such as described may be in one, two, or three of the asparagine-glycine pairs found in the encoded AAV amino acid sequence. In certain embodiments, such modifications are not made in all four of the asparagine-glycine pairs. Thus, a method for reducing deamidation of AAV and/or engineered AAV variants having lower deamidation rates. Additionally, or alternative one or more other amide amino acids may be changed to a non-amide amino acid to reduce deamidation of the AAV. In certain embodiments, a mutant AAV capsid as described herein contains a mutation in an asparagine-glycine pair, such that the glycine is changed to an alanine or a serine. A mutant AAV capsid may contain one, two or three mutants where the reference AAV natively contains four NG pairs. In certain embodiments, an AAV capsid may contain one, two, three or four such mutants where the reference AAV natively contains five NG pairs. In certain embodiments, a mutant AAV capsid contains only a single mutation in an NG pair. In certain embodiments, a mutant AAV capsid contains mutations in two different NG pairs. In certain embodiments, a mutant AAV capsid contains mutation is two different NG pairs which are located in structurally separate location in the AAV capsid. In certain embodiments, the mutation is not in the VP1-unique region. In certain embodiments, one of the mutations is in the VP1-unique region. Optionally, a mutant AAV capsid contains no modifications in the NG pairs, but contains mutations to minimize or eliminate deamidation in one or more asparagines, or a glutamine, located outside of an NG pair. In the AAVhu68 capsid protein, 4 residues (N57, N329, N452, N512) routinely display levels of deamidation>70% and it most cases >90% across various lots. Additional asparagine residues (N94, N253, N270, N304, N409, N477, and Q599) also display deamidation levels up to −20% across various lots. The deamidation levels were initially identified using a trypsin digest and verified with a chymotrypsin digestion.

The AAVhu68 capsid contains subpopulations within the vp1 proteins, within the vp2 proteins and within the vp3 proteins which have modifications from the predicted amino acid residues in SEQ ID NO: 2. These subpopulations include, at a minimum, certain deamidated asparagine (N or Asn) residues. For example, certain subpopulations comprise at least one, two, three or four highly deamidated asparagines (N) positions in asparagine-glycine pairs in SEQ ID NO: 2 and optionally further comprising other deamidated amino acids, wherein the deamidation results in an amino acid change and other optional modifications. The various combinations of these and other modifications are described herein.

In certain embodiments, the rAAV as described herein is a self-complementary AAV. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

The recombinant adeno-associated virus (AAV) described herein may be generated using techniques which are known. See, e.g., WO 2003/042397; WO 2005/033321, WO 2006/110689; U.S. Pat. No. 7,588,772 B2. Such a method involves culturing a host cell which contains a nucleic acid sequence encoding an AAV capsid; a functional rep gene; an expression cassette as described herein flanked by AAV inverted terminal repeats (ITRs); and sufficient helper functions to permit packaging of the expression cassette into the AAV capsid protein. Also provided herein is the host cell which contains a nucleic acid sequence encoding an AAV capsid; a functional rep gene; a vector genome as described; and sufficient helper functions to permit packaging of the vector genome into the AAV capsid protein. In one embodiment, the host cell is a HEK 293 cell. These methods are described in more detail in WO2017160360 A2, which is incorporated by reference herein.

Other methods of producing rAAV available to one of skill in the art may be utilized. Suitable methods may include without limitation, baculovirus expression system or production via yeast. See, e.g., Robert M. Kotin, Large-scale recombinant adeno-associated virus production. Hum Mol Genet. 2011 Apr. 15; 20(R1): R2-R6. Published online 2011 Apr. 29. doi: 10.1093/hmg/ddr141; Aucoin M G et al., Production of adeno-associated viral vectors in insect cells using triple infection: optimization of baculovirus concentration ratios. Biotechnol Bioeng. 2006 Dec. 20; 95(6):1081-92; SAMI S. THAKUR, Production of Recombinant Adeno-associated viral vectors in yeast. Thesis presented to the Graduate School of the University of Florida, 2012; Kondratov O et al. Direct Head-to-Head Evaluation of Recombinant Adeno-associated Viral Vectors Manufactured in Human versus Insect Cells, Mol Ther. 2017 Aug. 10. pii: S1525-0016(17)30362-3. doi: 10.1016/j.ymthe.2017.08.003. [Epub ahead of print]; Mietzsch M et al, OneBac 2.0: Sf9 Cell Lines for Production of AAV1, AAV2, and AAV8 Vectors with Minimal Encapsidation of Foreign DNA. Hum Gene Ther Methods. 2017 February; 28(1):15-22. doi: 10.1089/hgtb.2016.164.; Li L et al. Production and characterization of novel recombinant adeno-associated virus replicative-form genomes: a eukaryotic source of DNA for gene transfer. PLoS One. 2013 Aug. 1; 8(8):e69879. doi: 10.1371/journal.pone.0069879. Print 2013; Galibert L et al, Latest developments in the large-scale production of adeno-associated virus vectors in insect cells toward the treatment of neuromuscular diseases. J Invertebr Pathol. 2011 July; 107 Suppl:580-93. doi: 10.1016/j.jip.2011.05.008; and Kotin R M, Large-scale recombinant adeno-associated virus production. Hum Mol Genet. 2011 Apr. 15; 20(R1):R2-6. doi: 10.1093/hmg/ddr141. Epub 2011 Apr. 29.

A two-step affinity chromatography purification at high salt concentration followed by anion exchange resin chromatography are used to purify the vector drug product and to remove empty capsids. These methods are described in more detail in WO 2017/160360 entitled “Scalable Purification Method for AAV9”, which is incorporated by reference herein. In brief, the method for separating rAAV9 particles having packaged genomic sequences from genome-deficient AAV9 intermediates involves subjecting a suspension comprising recombinant AAV9 viral particles and AAV 9 capsid intermediates to fast performance liquid chromatography, wherein the AAV9 viral particles and AAV9 intermediates are bound to a strong anion exchange resin equilibrated at a pH of 10.2, and subjected to a salt gradient while monitoring eluate for ultraviolet absorbance at about 260 and about 280. Although less optimal for rAAV9, the pH may be in the range of about 10.0 to 10.4. In this method, the AAV9 full capsids are collected from a fraction which is eluted when the ratio of A260/A280 reaches an inflection point. In one example, for the Affinity Chromatography step, the diafiltered product may be applied to a Capture Select™ Poros-AAV2/9 affinity resin (Life Technologies) that efficiently captures the AAV2/9 serotype. Under these ionic conditions, a significant percentage of residual cellular DNA and proteins flow through the column, while AAV particles are efficiently captured.

Conventional methods for characterization or quantification of rAAV are available to one of skill in the art. To calculate empty and full particle content, VP3 band volumes for a selected sample (e.g., in examples herein an iodixanol gradient-purified preparation where # of GC=# of particles) are plotted against GC particles loaded. The resulting linear equation (y=mx+c) is used to calculate the number of particles in the band volumes of the test article peaks. The number of particles (pt) per 20 μl loaded is then multiplied by 50 to give particles (pt)/mL. Pt/mL divided by GC/mL gives the ratio of particles to genome copies (pt/GC). Pt/mL−GC/mL gives empty pt/mL. Empty pt/mL divided by pt/mL and ×100 gives the percentage of empty particles. Generally, methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Viral. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e. SYPRO ruby or Coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is used which utilizes a broad-spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2 fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000 fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April; 25(2):115-25. doi: 10.1089/hgtb.2013.131. Epub 2014 Feb. 14.

Methods for determining the ratio among vp1, vp2, and vp3 of capsid protein are also available. See, e.g., Vamseedhar Rayaprolu et al, Comparative Analysis of Adeno-Associated Virus Capsid Stability and Dynamics, J Virol. 2013 December; 87(24): 13150-13160; Buller R M, Rose J A. 1978. Characterization of adenovirus-associated virus-induced polypeptides in KB cells. J. Virol. 25:331-338; and Rose J A, Maizel J V, Inman J K, Shatkin A J. 1971. Structural proteins of adenovirus-associated viruses. J. Virol. 8:766-770.

It should be understood that the compositions in the rAAV described herein are intended to be applied to other compositions, regimens, aspects, embodiments, and methods described across the Specification.

### Pharmaceutical Composition

A pharmaceutical composition comprising an hGAA780I fusion protein or an expression cassette comprising the hGAA780I fusion protein transgene may be a liquid suspension, a lyophilized or frozen composition, or another suitable formulation. In certain embodiments, the composition comprises hGAA780I fusion protein or an expression cassette and a physiologically compatible liquid (e.g., a solution, diluent, carrier) which forms a suspension. Such a liquid is preferably aqueous based and may contain one or more: buffering agent(s), surfactant(s), pH adjuster(s), preservative(s), or other suitable excipients. Suitable components are discussed in more detail below. The pharmaceutical composition comprises the aqueous suspending liquid and any selected excipients, and a hGAA780I fusion protein or the expression cassette.

In certain embodiments, the pharmaceutical composition comprises the expression cassette comprising the transgene and a non-viral delivery system. This may include, e.g., naked DNA, naked RNA, an inorganic particle, a lipid or lipid-like particle, a chitosan-based formulation and others known in the art and described for example by Ramamoorth and Narvekar, as cited above). In other embodiments, the pharmaceutical composition is a suspension comprising the expression cassette comprising the transgene engineered in a viral vector system. In certain embodiments, the pharmaceutical composition comprises a non-replicating viral vector. Suitable viral vectors may include any suitable delivery vector, such as, e.g., a recombinant adenovirus, a recombinant lentivirus, a recombinant bocavirus, a recombinant adeno-associated virus (AAV), or another recombinant parvovirus. In certain embodiments, the viral vector is a recombinant AAV for delivery of a gene product to a patient in need thereof.

In one embodiment, the pharmaceutical composition comprises a hGAA780I fusion protein or an expression cassette comprising the coding sequences for the hGAA780I fusion protein and a formulation buffer suitable for delivery via intracerebroventricular (ICV), intrathecal (IT), intracisternal, or intravenous (IV) injection. In one embodiment, the expression cassette is part of a vector genome packaged a recombinant viral vector (i.e., an rAAV.hGAA780I carrying a fusion protein).

In one embodiment, the pharmaceutical composition comprises a hGAA780I fusion protein, or a functional fragment thereof, for delivery to a subject as an enzyme replacement therapy (ERT). Such pharmaceutical compositions are usually administered intravenously, however intradermal, intramuscular or oral administration is also possible in some circumstances. The compositions can be administered for prophylactic treatment of individuals suffering from, or at risk of, Pompe disease. For therapeutic applications, the pharmaceutical compositions are administered to a patient suffering from established disease in an amount sufficient to reduce the concentration of accumulated metabolite and/or prevent or arrest further accumulation of metabolite. For individuals at risk of lysosomal enzyme deficiency disease, the pharmaceutical compositions are administered prophylactically in an amount sufficient to either prevent or inhibit accumulation of metabolite. The modified GAA compositions described herein are administered in a therapeutically effective amount. In general, a therapeutically effective amount can vary depending on the severity of the medical condition in the subject, as well as the subject's age, general condition, and gender. Dosages can be determined by the physician and can be adjusted as necessary to suit the effect of the observed treatment. In one aspect, provided herein is a pharmaceutical composition for ERT formulated to contain a unit dosage of a hGAA780I fusion protein, or functional fragment thereof.

In one embodiment, a composition includes a final formulation suitable for delivery to a subject, e.g., is an aqueous liquid suspension buffered to a physiologically compatible pH and salt concentration. Optionally, one or more surfactants are present in the formulation. In another embodiment, the composition may be transported as a concentrate which is diluted for administration to a subject. In other embodiments, the composition may be lyophilized and reconstituted at the time of administration.

In one embodiment, a composition as provided herein comprises a surfactant, preservative, excipients, and/or buffer dissolved in the aqueous suspending liquid. In one embodiment, the buffer is PBS. In another embodiment, the buffer is an artificial cerebrospinal fluid (aCSF), e.g., Eliott's formulation buffer; or Harvard apparatus perfusion fluid (an artificial CSF with final Ion Concentrations (in mM): Na 150; K 3.0; Ca 1.4; Mg 0.8; P 1.0; Cl 155). Various suitable solutions are known including those which include one or more of: buffering saline, a surfactant, and a physiologically compatible salt or mixture of salts adjusted to an ionic strength equivalent to about 100 mM sodium chloride (NaCl) to about 250 mM sodium chloride, or a physiologically compatible salt adjusted to an equivalent ionic concentration.

Suitably, the formulation is adjusted to a physiologically acceptable pH, e.g., in the range of pH 6 to 8, or pH 6.5 to 7.5, pH 7.0 to 7.7, or pH 7.2 to 7.8. As the pH of the cerebrospinal fluid is about 7.28 to about 7.32, for intrathecal delivery, a pH within this range may be desired; whereas for intravenous delivery, a pH of 6.8 to about 7.2 may be desired. However, other pHs within the broadest ranges and these subranges may be selected for other route of delivery.

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly (propylene oxide)) flanked by two hydrophilic chains of polyoxyethylene (poly (ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

In one example, the formulation may contain, e.g., buffered saline solution comprising one or more of sodium chloride, sodium bicarbonate, dextrose, magnesium sulfate (e.g., magnesium sulfate.7H2O), potassium chloride, calcium chloride (e.g., calcium chloride.2H2O), dibasic sodium phosphate, and mixtures thereof, in water. Suitably, for intrathecal delivery, the osmolarity is within a range compatible with cerebrospinal fluid (e.g., about 275 to about 290); see, e.g., emedicine.medscape.com/article/2093316-overview. Optionally, for intrathecal delivery, a commercially available diluent may be used as a suspending agent, or in combination with another suspending agent and other optional excipients. See, e.g., Elliotts B® solution [Lukare Medical].

In other embodiments, the formulation may contain one or more permeation enhancers. Examples of suitable permeation enhancers may include, e.g., mannitol, sodium glycocholate, sodium taurocholate, sodium deoxycholate, sodium salicylate, sodium caprylate, sodium caprate, sodium lauryl sulfate, polyoxyethylene-9-laurel ether, or EDTA.

Additionally provided is a pharmaceutical composition comprising a pharmaceutically acceptable carrier and a vector comprising a nucleic acid sequence as described herein. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. Delivery vehicles such as liposomes, nanocapsules, microparticles, microspheres, lipid particles, vesicles, and the like, may be used for the introduction of the compositions of described herein into suitable host cells. In particular, the rAAV vector may be formulated for delivery either encapsulated in a lipid particle, a liposome, a vesicle, a nanosphere, or a nanoparticle or the like. In one embodiment, a therapeutically effective amount of the vector is included in the pharmaceutical composition. The selection of the carrier is not a limitation of the present invention. Other conventional pharmaceutically acceptable carrier, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The phrase “pharmaceutically acceptable” refers to molecular entities and compositions that do not produce an allergic or similar untoward reaction when administered to a host.

As used herein, the term “dosage” or “amount” can refer to the total dosage or amount delivered to the subject in the course of treatment, or the dosage or amount delivered in a single unit (or multiple unit or split dosage) administration.

The aqueous suspension or pharmaceutical compositions described herein are designed for delivery to subjects in need thereof by any suitable route or a combination of different routes. In one embodiment, the pharmaceutical composition is formulated for delivery via intracerebroventricular (ICV), intrathecal (IT), or intracisternal injection. In one embodiment, the compositions described herein are designed for delivery to subjects in need thereof by intravenous injection. Alternatively, other routes of administration may be selected (e.g., oral, inhalation, intranasal, intratracheal, intraarterial, intraocular, intramuscular, and other parenteral routes).

As used herein, the terms “intrathecal delivery” or “intrathecal administration” refer to a route of administration for drugs via an injection into the spinal canal, more specifically into the subarachnoid space so that it reaches the cerebrospinal fluid (CSF). Intrathecal delivery may include lumbar puncture, intraventricular, suboccipital/intracisternal, and/or C1-2 puncture. For example, material may be introduced for diffusion throughout the subarachnoid space by means of lumbar puncture. In another example, injection may be into the cisterna magna. Intracisternal delivery may increase vector diffusion and/or reduce toxicity and inflammation caused by the administration. See, e.g., Christian Hinderer et al, Widespread gene transfer in the central nervous system of cynomolgus macaques following delivery of AAV9 into the cisterna magna, Mol Ther Methods Clin Dev. 2014; 1: 14051. Published online 2014 Dec. 10. doi: 10.1038/mtm.2014.51.

As used herein, the terms “intracisternal delivery” or “intracisternal administration” refer to a route of administration for drugs directly into the cerebrospinal fluid of the brain ventricles or within the cisterna magna cerebellomedularis, more specifically via a suboccipital puncture or by direct injection into the cisterna magna or via permanently positioned tube.

In one aspect, provided herein is a pharmaceutical composition comprising a vector as described herein in a formulation buffer. In certain embodiments, the replication-defective virus compositions can be formulated in dosage units to contain an amount of replication-defective virus that is in the range of about 1.0×10⁹ GC to about 1.0×10¹⁶ GC (to treat an average subject of 70 kg in body weight) including all integers or fractional amounts within the range, and preferably 1.0×10¹² GC to 1.0×10¹⁴ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 1×10¹⁰ to about 1×10¹² GC per dose including all integers or fractional amounts within the range.

In one embodiment, provided is a pharmaceutical composition comprising a rAAV as described herein in a formulation buffer. In one embodiment, the rAAV is formulated at about 1×10⁹ genome copies (GC)/mL to about 1×10¹⁴ GC/mL. In a further embodiment, the rAAV is formulated at about 3×10⁹ GC/mL to about 3×10¹³ GC/mL. In yet a further embodiment, the rAAV is formulated at about 1×10⁹ GC/mL to about 1×10¹³ GC/mL. In one embodiment, the rAAV is formulated at least about 1×10¹¹ GC/mL.

In one embodiment, the pharmaceutical composition comprising a rAAV as described herein is administrable at a dose of about 1×10⁹ GC per gram of brain mass to about 1×10¹⁴ GC per gram of brain mass.

It should be understood that the compositions in the pharmaceutical compositions described herein are intended to be applied to other compositions, regimens, aspects, embodiments, and methods described across the Specification.

### Method of Treatment

A therapeutic regimen for treating a patient having Pompe disease is provided which comprises an expression cassette, an rAAV, and/or hGAA780I fusion protein as described herein, optionally in combination with an immunomodulator. In certain embodiments, the patient has late onset Pompe disease. In other embodiments, the patient has childhood onset Pompe disease. In certain embodiments, a co-therapeutic is delivered with the expression cassette, rAAV, or hGAA780I fusion protein such as an immunomodulatory regimen. Additionally, or alternatively, the co-therapy may include one or more of a bronchodilator, an acetylcholinesterase inhibitor, respiratory muscle strength training (RMST), enzyme replacement therapy, and/or diaphragmatic pacing therapy. In certain embodiments, the patient receives a single administration of an rAAV. In certain embodiments, the patient receives a single administration of a composition comprising an expression cassette and/or an rAAV as described herein. In certain embodiments, this single administration of a composition comprising an effective amount of an expression cassette involves at least one co-therapeutic. In certain embodiments, a patient is administered an expression cassette, rAAV, and/or hGAA780I fusion protein or as described herein via two different routes at substantially the same time. In certain embodiments, the two different routes of injection are intravenous and intrathecal administration. In one embodiment, the composition is a suspension is delivered to the subject intracerebroventricularly, intrathecally, intracisternally, or intravenously. In certain embodiments, a patient having a deficiency in alpha-glucosidase is administered a composition as provided herein to improve one or more of cardiac, respiratory, and/or skeletal muscle function. In certain embodiments, there is reduced glycogen storage and/or autophagic buildup in one or more of the heart, CNS (brain), and/or skeletal muscle as a result of treatment.

In certain embodiments, an expression cassette, rAAV, viral or non-viral vector is used in preparing a medicament. In certain embodiments, use of a composition for treating Pompe disease is provided.

These compositions may be used in combination with other therapies, including, e.g., immunotherapies, enzyme replacement therapy (e.g., Lumizyme, marketed by Genzyme, a Sanofi Corporation, and as Myozyme outside the United States). Additional treatment of Pompe disease is symptomatic and supportive. For example, respiratory support may be required; physical therapy may be helpful to strengthen respiratory muscles; some patients may need respiratory assistance through mechanical ventilation (i.e. bipap or volume ventilators) during the night and/or periods of the day. In addition, it may be necessary for additional support during respiratory tract infections. Orthopedic devices including braces may be recommended for some patients. Surgery may be required for certain orthopedic symptoms such as contractures or spinal deformity. Some infants may require the insertion of a feeding tube that is run through the nose, down the esophagus and into the stomach (nasogastric tube). In some children, a feeding tube may need to be inserted directly into the stomach through a small surgical opening in the abdominal wall. Some individuals with late onset Pompe disease may require a soft diet, but few require feeding tubes.

As described herein, the terms “increase” (e.g., increasing hGAA levels following treatment with hGAA780I fusion protein as measured in tissue, blood, etc.) or “decrease”, “reduce”, “ameliorate”, “improve”, “delay”, or any grammatical variation thereof, or any similar terms indicating a change, mean a variation of about 5 fold, about 2 fold, about 1 fold, about 90%, about 80%, about 70%, about 60%, about 50%, about 40%, about 30%, about 20%, about 10%, or about 5% compared to the corresponding reference (e.g., untreated control or a subject in normal condition without Pompe), unless otherwise specified.

“Patient” or “subject”, as used herein interchangeably, means a male or female mammalian animal, including a human, a veterinary or farm animal, a domestic animal or pet, and animals normally used for clinical research. In one embodiment, the subject of these methods and compositions is a human patient. In one embodiment, the subject of these methods and compositions is a male or female human.

In one embodiment, the suspension has a pH of about 7.28 to about 7.32.

Suitable volumes for delivery of these doses and concentrations may be determined by one of skill in the art. For example, volumes of about 1 μL to 150 mL may be selected, with the higher volumes being selected for adults. Typically, for newborn infants a suitable volume is about 0.5 mL to about 10 mL, for older infants, about 0.5 mL to about 15 mL may be selected. For toddlers, a volume of about 0.5 mL to about 20 mL may be selected. For children, volumes of up to about 30 mL may be selected. For pre-teens and teens, volumes up to about 50 mL may be selected. In still other embodiments, a patient may receive an intrathecal administration in a volume of about 5 mL to about 15 mL are selected, or about 7.5 mL to about 10 mL. Other suitable volumes and dosages may be determined. The dosage will be adjusted to balance the therapeutic benefit against any side effects and such dosages may vary depending upon the therapeutic application for which the recombinant vector is employed.

In one embodiment, the composition comprising an rAAV as described herein is administrable at a dose of about 1×10⁹ GC per gram of brain mass to about 1×10¹⁴ GC per gram of brain mass. In certain embodiments, the rAAV is co-administered systemically at a dose of about 1×10⁹ GC per kg body weight to about 1×10¹³ GC per kg body weight.

In one embodiment, the subject is delivered a therapeutically effective amount of the expression cassette, rAAV or hGAA780I fusion protein described herein. As used herein, a “therapeutically effective amount” refers to the amount of the expression cassette, rAAV, or hGAA780I fusion protein, or a combination thereof. Thus, in certain embodiments, the method comprises administering to a subject a rAAV or expression cassette for delivery of an hGAA780I fusion protein-encoding nucleic acid sequence in combination with administering a composition comprising an hGAA780I fusion protein enzyme provided herein.

In one embodiment, the expression cassette is in a vector genome delivered in an amount of about 1×10⁹ GC per gram of brain mass to about 1×10¹³ genome copies (GC) per gram (g) of brain mass, including all integers or fractional amounts within the range and the endpoints. In another embodiment, the dosage is 1×10¹⁰ GC per gram of brain mass to about 1×10¹³ GC per gram of brain mass. In specific embodiments, the dose of the vector administered to a patient is at least about 1.0×10⁹ GC/g, about 1.5×10⁹ GC/g, about 2.0×10⁹ GC/g, about 2.5×10⁹ GC/g, about 3.0×10⁹ GC/g, about 3.5×10⁹ GC/g, about 4.0×10⁹ GC/g, about 4.5×10⁹ GC/g, about 5.0×10⁹ GC/g, about 5.5×10⁹ GC/g, about 6.0×10⁹ GC/g, about 6.5×10⁹ GC/g, about 7.0×10⁹ GC/g, about 7.5×10⁹ GC/g, about 8.0×10⁹ GC/g, about 8.5×10⁹ GC/g, about 9.0×10⁹ GC/g, about 9.5×10⁹ GC/g, about 1.0×10¹⁰ GC/g, about 1.5×10¹⁰ GC/g, about 2.0×10¹⁰ GC/g, about 2.5×10¹⁰ GC/g, about 3.0×10¹⁰ GC/g, about 3.5×10¹⁰ GC/g, about 4.0×10¹⁰ GC/g, about 4.5×10¹⁰ GC/g, about 5.0×10¹⁰ GC/g, about 5.5×10¹⁰ GC/g, about 6.0×10¹⁰ GC/g, about 6.5×10¹⁰ GC/g, about 7.0×10¹⁰ GC/g, about 7.5×10¹⁰ GC/g, about 8.0×10¹⁰ GC/g, about 8.5×10¹⁰ GC/g, about 9.0×10¹⁰ GC/g, about 9.5×10¹⁰ GC/g, about 1.0×10¹¹ GC/g, about 1.5×10¹¹ GC/g, about 2.0×10¹¹ GC/g, about 2.5×10¹¹ GC/g, about 3.0×10¹¹ GC/g, about 3.5×10¹¹ GC/g, about 4.0×10¹¹ GC/g, about 4.5×10₁₁ GC/g, about 5.0×10¹¹ GC/g, about 5.5×10¹¹ GC/g, about 6.0×10¹¹ GC/g, about 6.5×10¹¹ GC/g, about 7.0×10¹¹ GC/g, about 7.5×10¹¹ GC/g, about 8.0×10¹¹ GC/g, about 8.5×10¹¹ GC/g, about 9.0×10¹¹ GC/g, about 9.5×10¹¹ GC/g, about 1.0×10¹² GC/g, about 1.5×10¹² GC/g, about 2.0×10¹² GC/g, about 2.5×10¹² GC/g, about 3.0×10¹² GC/g, about 3.5×10¹² GC/g, about 4.0×10¹² GC/g, about 4.5×10¹² GC/g, about 5.0×10¹² GC/g, about 5.5×10¹² GC/g, about 6.0×10¹² GC/g, about 6.5×10¹² GC/g, about 7.0×10¹² GC/g, about 7.5×10¹² GC/g, about 8.0×10¹² GC/g, about 8.5×10¹² GC/g, about 9.0×10¹² GC/g, about 9.5×10¹² GC/g, about 1.0×10¹³ GC/g, about 1.5×10¹³ GC/g, about 2.0×10¹³ GC/g, about 2.5×10¹³ GC/g, about 3.0×10¹³ GC/g, about 3.5×10¹³ GC/g, about 4.0×10¹³ GC/g, about 4.5×10¹³ GC/g, about 5.0×10¹³ GC/g, about 5.5×10¹³ GC/g, about 6.0×10¹³ GC/g, about 6.5×10¹³ GC/g, about 7.0×10¹³ GC/g, about 7.5×10¹³ GC/g, about 8.0×10¹³ GC/g, about 8.5×10¹³ GC/g, about 9.0×10¹³ GC/g, about 9.5×10¹³ GC/g, or about 1.0×10¹⁴ GC/g brain mass.

In one embodiment, the method of treatment comprises delivery of the hGAA780I fusion protein as an enzyme replacement therapy. In certain embodiments, hGAA780I fusion protein is delivered as an ERT in combination with a gene therapy (including but not limited to an expression cassette or an rAAV as provided herein). In certain embodiments, the method comprises administering to a subject more than one ERT (e.g. a composition comprising hGAA780I fusion protein in combination with another therapeutic protein, such as Lumizyme). A composition comprising a hGAA780I fusion protein described herein may be administered to a subject every 1, 2, 3, 4, 5, 6, 7, 8, 9, 10, or more days. Administration may be by intravenous infusion to an outpatient, prescribed weekly, monthly, or bimonthly administration. Appropriate therapeutically effective dosages of the compounds are selected by the treating clinician and include from about 1 μg/kg to about 500 mg/kg, from about 10 mg/kg to about 100 mg/kg, from about 20 mg/kg to about 100 mg/kg and approximately 20 mg/kg to approximately 50 mg/kg. In some embodiments, a suitable therapeutic dose is selected from, for example, 0.1, 0.25, 0.5, 0.75, 1, 5, 10, 15, 20, 30, 40, 50, 60, 70, and 100 mg/kg.

In certain embodiments, the method comprises administering hGAA780I fusion protein to a subject at a dosage of 10 mg/kg patient body weight or more per week to a patient. Often dosages are greater than 10 mg/kg per week. Dosages regimes can range from 10 mg/kg per week to at least 1000 mg/kg per week. Typically dosage regimes are 10 mg/kg per week, 15 mg/kg per week, 20 mg/kg per week, 25 mg/kg per week, 30 mg/kg per week, 35 mg/kg per week, 40 mg/kg week, 45 mg/kg per week, 60 mg/kg week, 80 mg/kg per week and 120 mg/kg per week. In preferred regimes, 10 mg/kg, 15 mg/kg, 20 mg/kg, 30 mg/kg or 40 mg/kg is administered once, twice, or three times weekly. Treatment is typically continued for at least 4 weeks, sometimes 24 weeks, and sometimes for the life of the patient. Optionally, levels of human alpha-glucosidase are monitored following treatment (e.g., in the plasma or muscle) and a further dosage is administered when detected levels fall substantially below (e.g., less than 20%) of values in normal persons. In one embodiment, hGAA780I is administered at an initially “high” dose (i.e., a “loading dose”), followed by administration of a lower doses (i.e., a “maintenance dose”). An example of a loading dose is at least about 40 mg/kg patient body weight 1 to 3 times per week (e.g., for 1, 2, or 3 weeks). An example of a maintenance dose is at least about 5 to at least about 10 mg/kg patient body weight per week, or more, such as 20 mg/kg per week, 30 mg/kg per week, 40 mg/kg week. In certain embodiments, a dosage is administered at increasing rate during the dosage period. Such can be achieved by increasing the rate of flow intravenous infusion or by using a gradient of increasing concentration of hGAA780I fusion protein administered at constant rate. Administration in this manner may reduce the risk of immunogenic reaction. In certain embodiments, the intravenous infusion occurs over a period of several hours (e.g., 1-10 hours and preferably 2-8 hours, more preferably 3-6 hours), and the rate of infusion is increased at intervals during the period of administration.

In one embodiment, the method further comprises the subject receives an immunosuppressive co-therapy. Immunosuppressants for such co-therapy include, but are not limited to, a glucocorticoid, steroids, antimetabolites, T-cell inhibitors, a macrolide (e.g., a rapamycin or rapalog), and cytostatic agents including an alkylating agent, an anti-metabolite, a cytotoxic antibiotic, an antibody, or an agent active on immunophilin. The immune suppressant may include a nitrogen mustard, nitrosourea, platinum compound, methotrexate, azathioprine, mercaptopurine, fluorouracil, dactinomycin, an anthracycline, mitomycin C, bleomycin, mithramycin, IL-2 receptor- or CD3-directed antibodies, anti-IL-2 antibodies, ciclosporin, tacrolimus, sirolimus, IFN-(3, IFN-γ, an opioid, or TNF-α (tumor necrosis factor-alpha) binding agent. In certain embodiments, the immunosuppressive therapy may be started 0, 1, 2, 7, or more days prior to the gene therapy administration. One or more of these drugs may be continued after gene therapy administration, at the same dose or an adjusted dose. Such therapy may be for about 1 week (7 days), about 60 days, or longer, as needed.

In one embodiment, a composition comprising the expression cassette as described herein is administrated once to the subject in need. In certain embodiments, the expression cassette is delivered via an rAAV. It should be understood that the compositions and the method described herein are intended to be applied to other compositions, regimens, aspects, embodiments and methods described across the specification.

The compositions and methods provided herein may be used to treat infantile onset-Pompe disease or late-onset Pompe disease and/or the symptoms associated therewith. In certain embodiments, efficacy can be determined by improvement of one or more symptoms of the disease or a slowing of disease progression. Symptoms of infantile onset-Pompe disease include, but are not limited to, hypotonia, respiratory/breathing problems, hepatomegaly, hypertrophic cardiomyopathy, as well as glycogen storage in heart, muscles, CNS (especially motor neurons). Symptoms of late onset-Pompe disease include, but are not limited to, proximal muscle weakness, respiratory/breathing problems, as well as glycogen storage in muscles and motor neurons. The route of administration may be determined based on a patient's condition and/or diagnosis. In certain embodiments, a method is provided for treatment of a patient diagnosed with infantile-onset Pompe disease or late-onset Pompe disease that includes administering a rAAV described herein for delivery of hGAA780I fusion protein via a combination of IV and ICM routes. In some embodiments, a patient identified as having late-onset Pompe disease is administered a treatment that includes only systemic delivery of a rAAV (e.g., only IV). As described herein, delivery of a composition comprising a rAAV can be in combination with enzyme replacement therapy (ERT). In certain embodiments, a method is provided for treating a subject diagnosed with Pompe disease that includes ICM delivery a rAAV described herein in combination with ERT. In certain embodiments, a subject identified as having infantile-onset Pompe disease is administered a rAAV described herein via ICM injection and also receives ERT for treatment of aspects of peripheral disease.

A “nucleic acid”, as described herein, can be RNA, DNA, or a modification thereof, and can be single or double stranded, and can be selected, for example, from a group including: nucleic acid encoding a protein of interest, oligonucleotides, nucleic acid analogues, for example peptide-nucleic acid (PNA), pseudocomplementary PNA (pc-PNA), locked nucleic acid (LNA) etc. Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, micro RNAi (mRNAi), antisense oligonucleotides etc.

Methods for “backtranslating” a protein, peptide, or polypeptide are known to those of skill in the art. Once the sequence of a protein is known, there are web-based and commercially available computer programs, as well as service-based companies which back translate the amino acids sequences to nucleic acid coding sequences. See, e.g., backtranseq by EMBOSS, (available online at ebi.ac.uk/Tools/st); Gene Infinity (available online at geneinfinity.org/sms/sms_-backtranslation.html); ExPasy (available online expasy.org/tools/). In one embodiment, the RNA and/or cDNA coding sequences are designed for optimal expression in human cells.

The term “percent (%) identity”, “sequence identity”, “percent sequence identity”, or “percent identical” in the context of nucleic acid sequences refers to the residues in the two sequences which are the same when aligned for correspondence. The length of sequence identity comparison may be over the full-length of the genome, the full-length of a gene coding sequence, or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired.

Percent identity may be readily determined for amino acid sequences over the full-length of a protein, polypeptide, about 32 amino acids, about 330 amino acids, or a peptide fragment thereof or the corresponding nucleic acid sequence coding sequences. A suitable amino acid fragment may be at least about 8 amino acids in length, and may be up to about 700 amino acids. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence.

Alignments are performed using any of a variety of publicly or commercially available Multiple Sequence Alignment Programs. Sequence alignment programs are available for amino acid sequences, e.g., the “Clustal X”, “Clustal Omega” “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thompson et al, Nucl. Acids. Res., 27(13):2682-2690 (1999).

Multiple sequence alignment programs are also available for nucleic acid sequences. Examples of such programs include, “Clustal W”, “Clustal Omega”, “CAP Sequence Assembly”, “BLAST”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference.

As used herein, the term “regulatory sequence”, or “expression control sequence” refers to nucleic acid sequences, such as initiator sequences, enhancer sequences, and promoter sequences, which induce, repress, or otherwise control the transcription of protein encoding nucleic acid sequences to which they are operably linked.

The term “exogenous” as used to describe a nucleic acid sequence or protein means that the nucleic acid or protein does not naturally occur in the position in which it exists in a chromosome, or host cell. An exogenous nucleic acid sequence also refers to a sequence derived from and inserted into the same host cell or subject, but which is present in a non-natural state, e.g. a different copy number, or under the control of different regulatory elements.

The term “heterologous” as used to describe a nucleic acid sequence or protein means that the nucleic acid or protein was derived from a different organism or a different species of the same organism than the host cell or subject in which it is expressed. The term “heterologous” when used with reference to a protein or a nucleic acid in a plasmid, expression cassette, or vector, indicates that the protein or the nucleic acid is present with another sequence or subsequence which with which the protein or nucleic acid in question is not found in the same relationship to each other in nature.

“Comprising” is a term meaning inclusive of other components or method steps. When “comprising” is used, it is to be understood that related embodiments include descriptions using the “consisting of” terminology, which excludes other components or method steps, and “consisting essentially of” terminology, which excludes any components or method steps that substantially change the nature of the embodiment or invention. It should be understood that while various embodiments in the specification are presented using “comprising” language, under various circumstances, a related embodiment is also described using “consisting of” or “consisting essentially of” language.

As used herein, the term “e” followed by a numerical (nn) value refers to an exponent and this term is used interchangeably with “×10 nn”. For example, 3e13 is equivalent to 3×10¹³.

It is to be noted that the term “a” or “an”, refers to one or more, for example, “a vector”, is understood to represent one or more vector(s). As such, the terms “a” (or “an”), “one or more,” and “at least one” is used interchangeably herein.

As used herein, the term “about” means a variability of plus or minus 10% from the reference given, unless otherwise specified.

### EXAMPLES

The invention is now described with reference to the following examples. These examples are provided for the purpose of illustration only and the invention should in no way be construed as being limited to these examples but rather should be construed to encompass any and all variations that become evident as a result of the teaching provided herein.

### Example 1: Materials and Methods

### Vector Production

The reference GAA sequence with a Val at 780, and the sequence with the V780I mutation were back-translated and the nucleotide sequence was engineered to generate cis-plasmids for AAV production with the expression cassettes under the CAG promoter. In addition, the cDNA sequence for the natural hGAA (reference sequence) was cloned into the same AAV-cis backbone for comparison with the non-engineered sequence. AAVhu68 vectors were produced and titrated by the Penn Vector Core as described before. (Lock, et al. 2010, Hum Gene Ther 21(10): 1259-1271). Briefly, HEK293 cells were triple-transfected and the culture supernatant was harvested, concentrated, and purified with an iodixanol gradient. The purified vectors were titrated with droplet digital PCR using primers targeting the rabbit Beta-globin polyA sequence as previously described (Lock, et al. (2014). Hum Gene Ther Methods 25(2): 115-125).

### Animals

### Mice

Pompe mice (Gaa knock-out (−/−), C57BL/6/129 background) founders were purchased from Jackson Labs (stock #004154, also known as 6neo mice). The breeding colony was maintained at the Gene Therapy Program AAALAC accredited barrier mouse facility, using heterozygote to heterozygote mating in order to produce null and WT controls within the same litters. Gaa knock-out mice are a widely used model for Pompe disease. They exhibit a progressive accumulation of lysosomal glycogen in heart, central nervous system, skeletal muscle, and diaphragm, with reduced mobility and progressive muscle weakness. The small size, reproducible phenotype, and efficient breeding allow for quick studies that are optimal for preclinical candidate in vivo screening.

Animal holding rooms were maintained at a temperature range of 64−79° F. (18-26° C.) with a humidity range of 30-70%.

Animals were housed with their parents and littermates until weaning and then in standard caging of two to five animals per cage in the Translational Research Laboratories (TRL) GTP vivarium. All cage sizes and housing conditions are in compliance with the Guide for the Care and Use of Laboratory Animals. Cages, water bottles, and bedding substrates are autoclaved into the barrier facility.

An automatically controlled 12-hour light/dark cycle was maintained. Each dark period began at 1900 hours (±30 minutes). Food was provided ad libitum (Purina, LabDiet®, 5053, Irradiated, PicoLab®, Rodent Diet 20, 251b). Water was accessible to all animals ad libitum via individually placed water bottle in each housing cage. At a minimum, water bottles were replaced once per week during weekly cage changing. The water supply was drawn from the City of Philadelphia and was chlorinated using a Getinge water purifier. Chlorination levels are tested daily by ULAR and maintained at 2-4 parts per million (ppm).

Nestlets™ were provided to each housing cage as enrichment.

### In Vivo Studies and Histology

Mice were administered a dose of 5×10¹¹ GCs (approximately 2.5×10¹³ GC/kg) or a dose of 5×10¹⁰ GCs (approximately 2.5×10¹² GC/kg) of AAVhu68.CAG.hGAA (various hGAA constructs) in 0.1 mL via the lateral tail vein (IV), were bled on Day 7 and Day 21 post vector dosing for serum isolation, and were terminally bled (for plasma isolation) and euthanized by exsanguination 28 days post-injection. Tissues were promptly collected, starting with the brain.

Organ list, necropsy

| | Flash frozen | |
|---|---|---|
| | (for protein | Formalin immersion |
| Tissue | extraction) | (for histology) |
| Plasma | X | |
| Left brain | | X |
| Right brain | X | |
| Cervical spinal cord | | X |
| Thoracic + Lumbar | X | |
| spinal cord | | |
| Heart | X | X |
| Liver | X | X |
| Diaphragm | X Right | X Left |
| Triceps muscle | X Right | X Left |
| Quadriceps muscle | X Right | X Left |
| Gastrocnemian muscle | X Right | X Left |
| Tibialis anterior muscle | X Right | X Left |

Tissues for histology were formalin-fixed and paraffin embedded using standard methods. Brain and spinal cord sections were stained with luxol fast blue (luxol fast blue stain kit, Abcam ab150675) and peripheral organs were stained with PAS (Periodic Acid-Schiff) using standard methods to detect polysaccharides such as glycogen in tissues. Immunostaining for hGAA was performed on formalin-fixed paraffin-embedded samples. Sections were deparaffinized, boiled in 10 mM citrate buffer (pH 6.0) for antigen retrieval, blocked with 1% donkey serum in PBS+0.2% Triton for 15 min, and then sequentially incubated with primary (Sigma HPA029126 anti-hGAA antibody) and biotinylated secondary antibodies diluted in blocking buffer; an HRP based colorimetric reaction was used to detect the signal.

Slides were reviewed in a blinded fashion by a board-certified Veterinary Pathologist. A semi-quantitative scoring system was established to measure the severity of the Pompe-related histological lesions in muscles (glycogen storage and autophagic buildup), as determined by the total percentage of cells presenting storage and/or vacuoles:

Histo scoring storage

| | |
|---|---|
| 0 | 0% |
| 1 | 1 to9% |
| 2 | 10 to 49% |
| 3 | 50 to 74% |
| 4 | 75 to 100% |

Vector related histopathological lesions were also estimated when applicable.

### Non-Human Primates

For vector administration, rhesus macaques were sedated with intramuscular dexmedetomidine and ketamine, and administered a single intra-cisterna magna (ICM) injection or intravenous injection. Needle placement for ICM injection was verified via myelography using a fluoroscope (OEC9800 C-Arm, GE), as previously described (Katz N, et al. Hum Gene Ther Methods. 2018 October; 29(5):212-219). Animals were euthanized by barbiturate overdose. Collected tissues were immediately frozen on dry ice or fixed in 10% formalin for histology.

Characterization of hGAA 780I Enzyme Performance In Vitro

### GAA Activity

Plasma or supernatant of homogenized tissues are mixed with 5.6 mM 4-MU-α-glucopyranoside pH 4.0 and incubated for three hours at 37° C. The reaction is stopped with 0.4 M sodium carbonate, pH 11.5. Relative fluorescence units, RFUs are measured using a Victor3 fluorimeter, ex 355 nm and emission at 460 nm. Activity in units of nmol/mL/hr are calculated by interpolation from a standard curve of 4-MU. Activity levels in individual tissue samples are normalized for total protein content in the homogenate supernatant. Equal volumes are used for plasma samples.

### GAA Signature Peptide by LC/MS

Plasma are precipitated in 100% methanol and centrifuged. Supernatants are discarded. The pellet is spiked with a stable isotope-labeled peptide unique to hGAA as an internal standard and resuspended with trypsin and incubated at 37° C. for one hour. The digestion is stopped with 10% formic acid. Peptides are separated by C-18 reverse phase chromatography and identified and quantified by ESI-mass spectroscopy. The total GAA concentration in plasma is calculated from the signature peptide concentration.

### Cell Surface Receptor Binding Assay

A 96-well plate is coated with receptor, washed, and blocked with BSA. CHO culture conditioned media or plasma containing equal activities of either rhGAA or engineered GAA is serially diluted three-fold to give a series of nine decreasing concentrations and incubated with co-coupled receptor. After incubation the plate is washed to remove any unbound GAA and 4-MU-α-glucopyranoside added for one hour at 37° C. The reaction is stopped with 1.0 M glycine, pH 10.5 and RFUs were read by a Spectramax fluorimeter; ex 370, emission 460. RFU's for each sample and are converted to nmol/mL/hr by interpolation from a standard curve of 4-MU. Nonlinear regression is done using GraphPad Prism.

### Glycogen-TFA Hydrolysis

Tissue homogenate is hydrolyzed with 4N TFA at 100° C. for four hours, dried and reconstituted in water. Hydrolyzed material is injected onto a CarboPac PA-10 2×250 mm column for glucose determination by high pH anion exchange chromatography with pulsed amerometric detection (HPAEC-PAD). The concentration of free glucose in each sample is calculated by interpolation from a glucose standard curve. Final data is reported as μg glycogen/mg protein.

### Example 2: Evaluation of rAAVhu68.hGAA Vectors in Pompe Mice

AAV vectors were diluted in sterile PBS for IV delivery to Pompe mice. Test articles included: AAVhu68.CAG.hGAAco.rBG, AAVhu68.CAG.hGAAcoV780LrBG, AAVhu68.CAG.BiP-vIGF2.hGAAco.rBG, AAVhu68.CAG.BiP-vIGF2.hGAAcoV780I.rBG, and AAVhu68.CAG.sp7co.Δ8.hGAAcoV780I.rBG. Wildtype and vehicle controls were included in the studies.

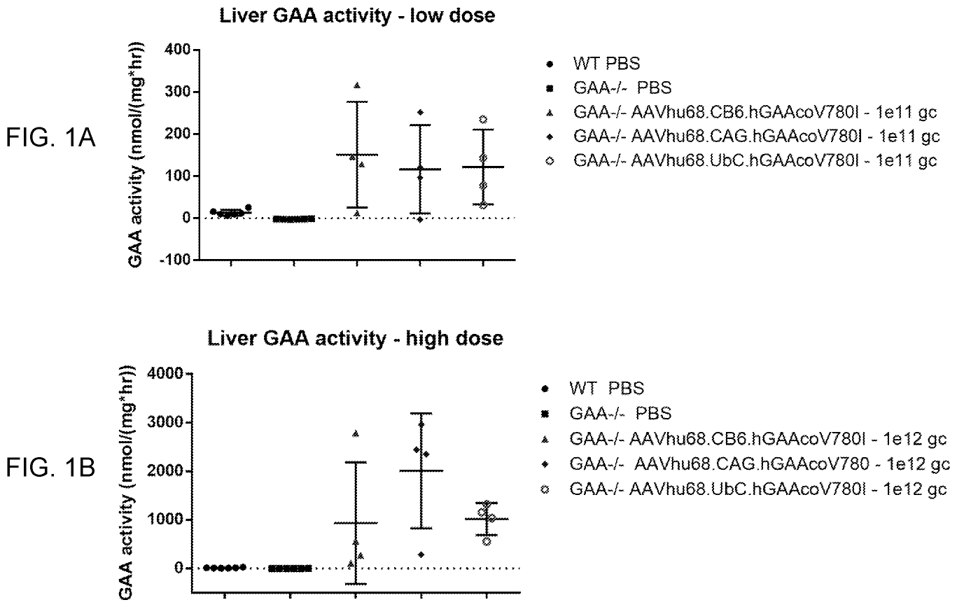

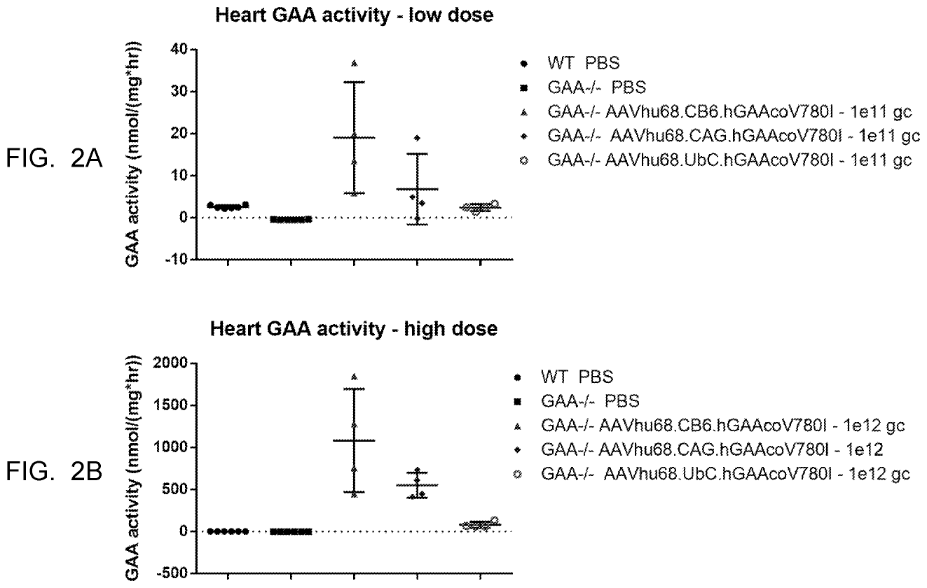

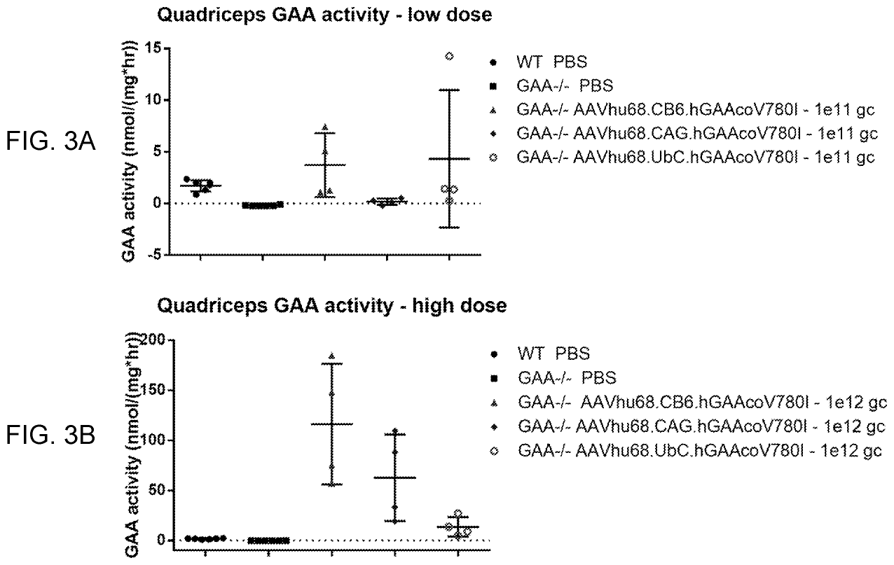

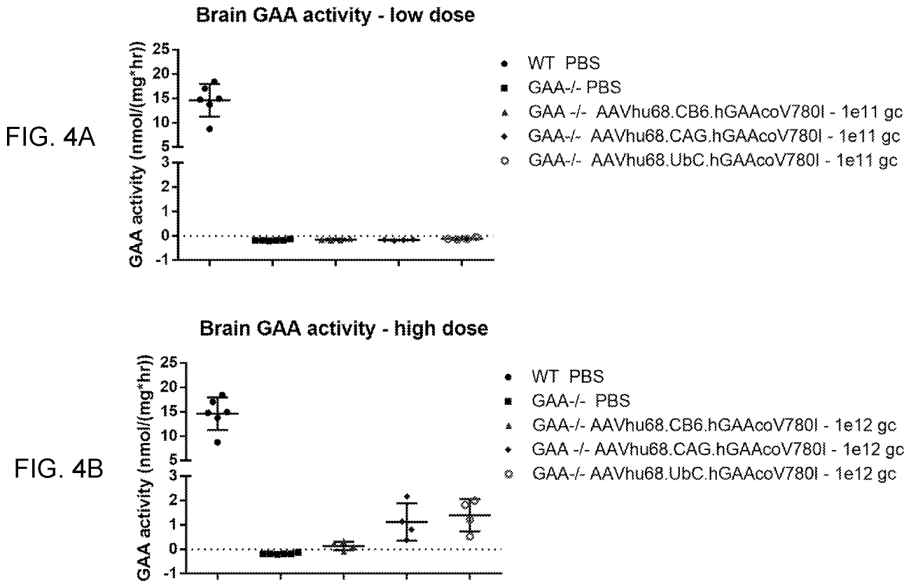

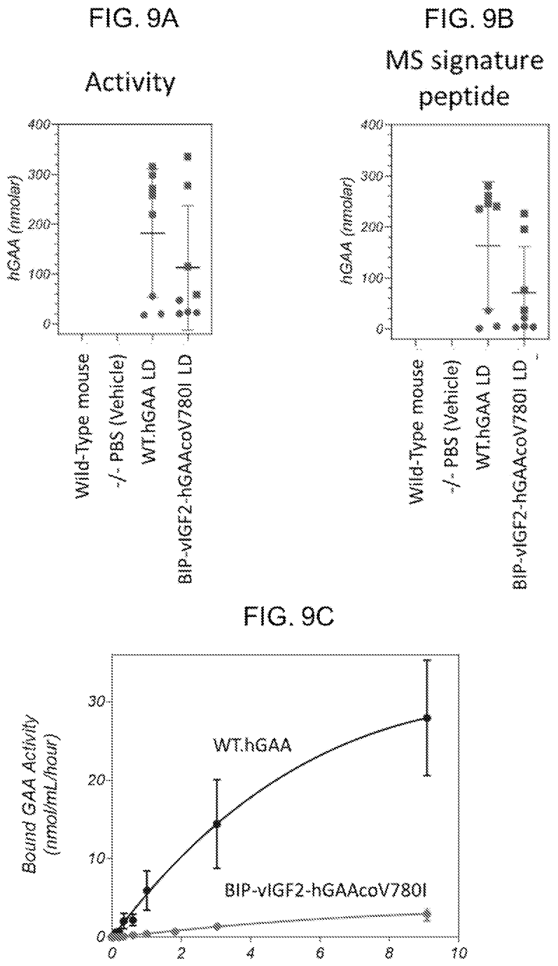

hGAA protein expression and activity were measured in various tissues collected from treated mice, including liver (FIG. 1A, FIG. 1B), heart (FIG. 2A, FIG. 2B), quadricep muscle (FIG. 3A, FIG. 3B), brain (FIG. 4A, FIG. 4B), plasma (FIG. 9A). All promoters performed equally well in the liver at both low and high doses. Administration of the vector expressing under the UbC promoter resulted in lower activity in skeletal muscle at both doses, and the vector with the CAG promoter had the best overall activity. The vector with the UbC promoter also had lower activity in the heart at both doses. Pompe mice vehicle (PBS) controls (FIG. 5D) displayed marked glycogen storage (dark staining on PAS stained sections) in the heart. Wildtype mice and all vector treated mice had near complete to complete clearance of storage. The two groups that received vectors encoding the hGAA reference sequence (V780), however, displayed moderate to marked fibrosing lymphocytic myocarditis (FIG. 5B and FIG. 5C), which was present in seven out of eight animals that received the hGAA native transgene and in three out of eight animals that received the engineered hGAA with BiP and vIGF2 modifications. Because none of the mice receiving the hGAAcoV780I enzyme had myocarditis (FIG. 5E, FIG. 5F, and FIG. 5G), this lesion was considered to be vector related and, more specifically, hGAA reference sequence specific.

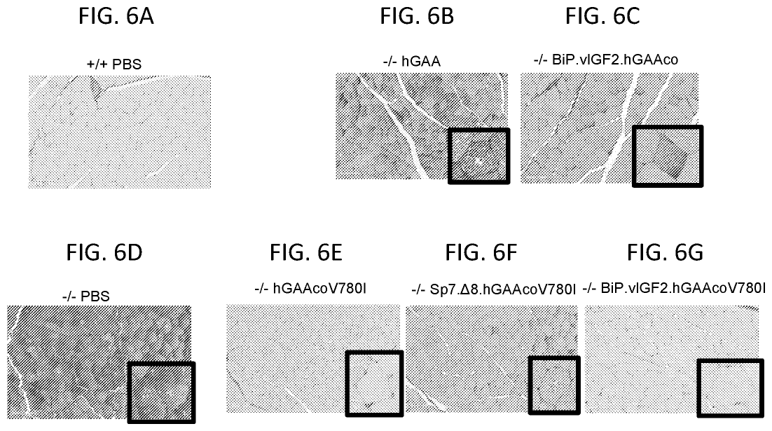

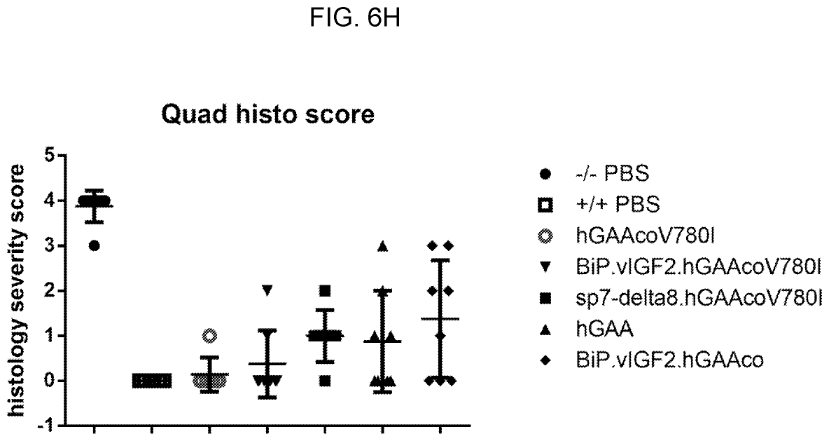

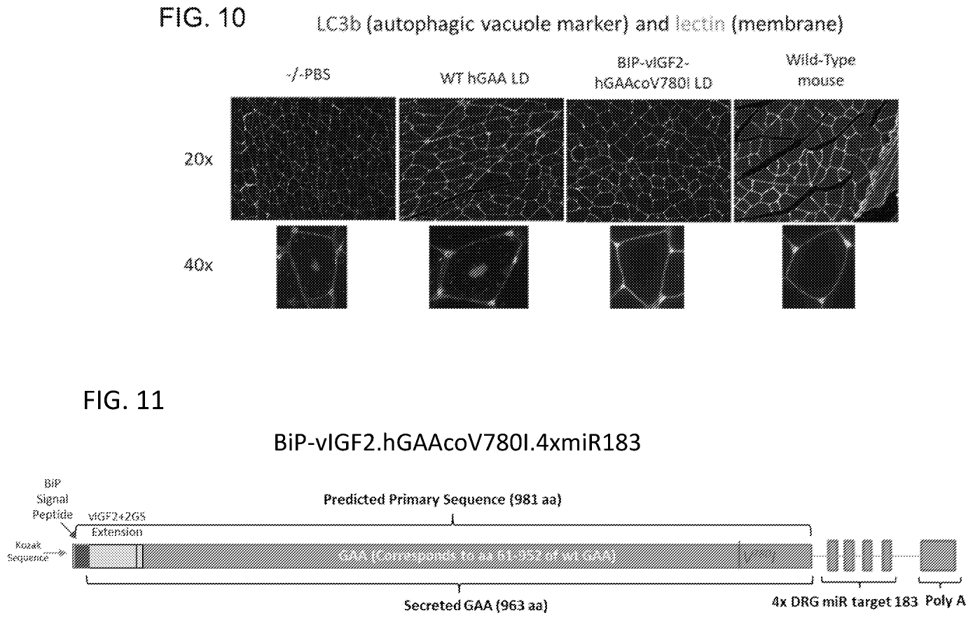

Analysis of quadricep tissue revealed that wildtype mice and all mice treated with vectors encoding the V780I variant, with or without further modification, had near complete to complete clearance of storage and autophagic buildup (FIG. 6A-FIG. 6H). The two groups receiving vectors encoding the reference sequence of hGAAV780 however displayed minimal to moderate glycogen storage remaining as well as autophagic buildup (FIG. 10), together demonstrating suboptimal correction of the two main hallmarks of Pompe disease. The best outcome was observed from delivery of the two vectors encoding the V780I variant, either in its native form or with the BiP-vIGF2 modifications. The sp7-delta8 modifications appeared to cause inconsistent correction of histological lesions attributed to Pompe disease. Both constructs encoding the reference hGAAV780 sequence were suboptimal at clearing glycogen storage and buildup.

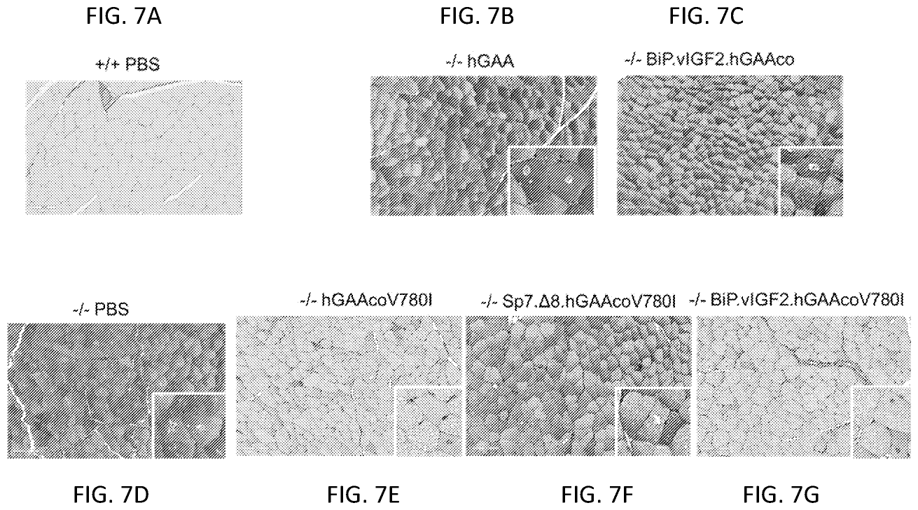

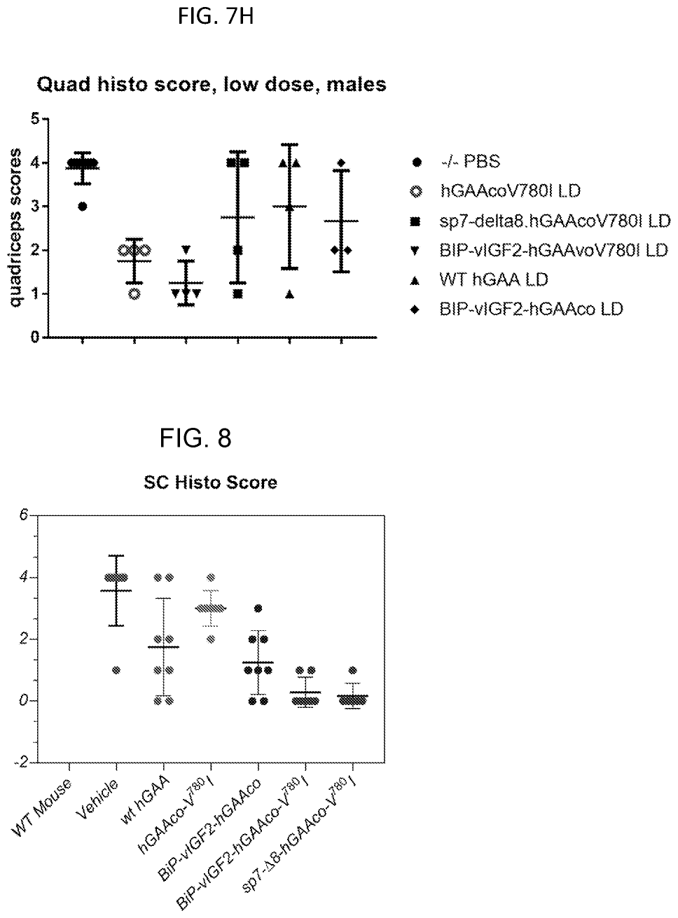

At high dose IV administration (5e11=2.5e13 GC/kg), hGAAcoV780I and BiP-vIGF2.hGAAcoV780I demonstrated near normal glycogen levels in quadriceps muscle and had markedly better hGAA uptake into cells (FIG. 7A-FIG. 7H). Evaluation of other skeletal muscles, including tibialis anterior (TA) and gastrocnemius, showed similar results (variant with V780I and cleared both glycogen and central autophagic vacuoles). All constructs reduced glycogen storage in heart, with BiP-vIGF2.hGAAcoV780I administration resulting in the lowest levels. Although glycogen levels in quadriceps muscle were near normal, PAS staining illustrated some differences, with hGAAcoV780I and BiP-vIGF2.hGAAcoV780I showing the best results.

At low dose IV administration (5e10=2.5e12 GC/kg), BiP-vIGF2.hGAAcoV780I demonstrated better glycogen reduction in heart and quadriceps muscle than hGAAcoV780I. Glycogen levels in brain and spinal cord were near normal with BiP-vIGF2.hGAAcoV780I, even with tissue levels of −15%, presumably due to better targeting. In the CNS, potent synergistic effects between the engineered construct and the V780I variant were observed. Only BiP-vIGF2.hGAAcoV780I cleared CNS glycogen.

As shown in FIG. 8, evaluation of spinal cord histology showed that mice treated with AAVhu68.BiP-vIGF2.hGAAcoV780I had near complete to complete clearance of glycogen storage, while mice treated with vectors encoding the reference hGAAV780 enzyme had remaining glycogen storage. Staining of brain sections also revealed correction with BiP-vIGF2.hGAAcoV780I, but not with the native hGAAV780 enzyme. The results demonstrate the contributions of both the V780I mutation and the BiP-vIGF2 modifications.

### Example 3: Effects of DRG-Detargeting on hGAA Expression in Pompe Mice

BiP-vIGF2.hGAAcoV780I was modified to include four mir183 target sites (BiP-vIGF2.hGAAcoV780I.4xmir183, SEQ ID NO: 30) (FIG. 11), packaged in an AAVhu68 capsid.

The vector genome contains the following sequence elements:

Inverted Terminal Repeats (ITRs): The ITRs are identical, reverse complementary sequences derived from AAV2 (130 bp, GenBank: NC_001401) that flank all components of the vector genome. The ITRs function as both the origin of vector DNA replication and the packaging signal for the vector genome when AAV and adenovirus helper functions are provided in trans. As such, the ITR sequences represent the only cis sequences required for vector genome replication and packaging.

CAG Promoter: Hybrid construct consisting of the cytomegalovirus (CMV) enhancer, the chicken beta-actin (CB) promoter (282 bp, GenBank: X00182.1), and a rabbit beta-globin intron.

Coding sequence: An engineered cDNA (nt 1141 to 4092 of SEQ ID NO: 30) encoding BiP-vIGF2.hGAAcoV780I (SEQ ID NO: 31).

miR target sequences: Four tandem miR-183 target sequences (SEQ ID NO: 26)

Rabbit β-Globin Polyadenylation Signal (rBG PolyA): The rBG PolyA signal (127 bp, GenBank: V00882.1) facilitates efficient polyadenylation of the transgene mRNA in cis. This element functions as a signal for transcriptional termination, a specific cleavage event at the 3′ end of the nascent transcript and the addition of a long polyadenyl tail.

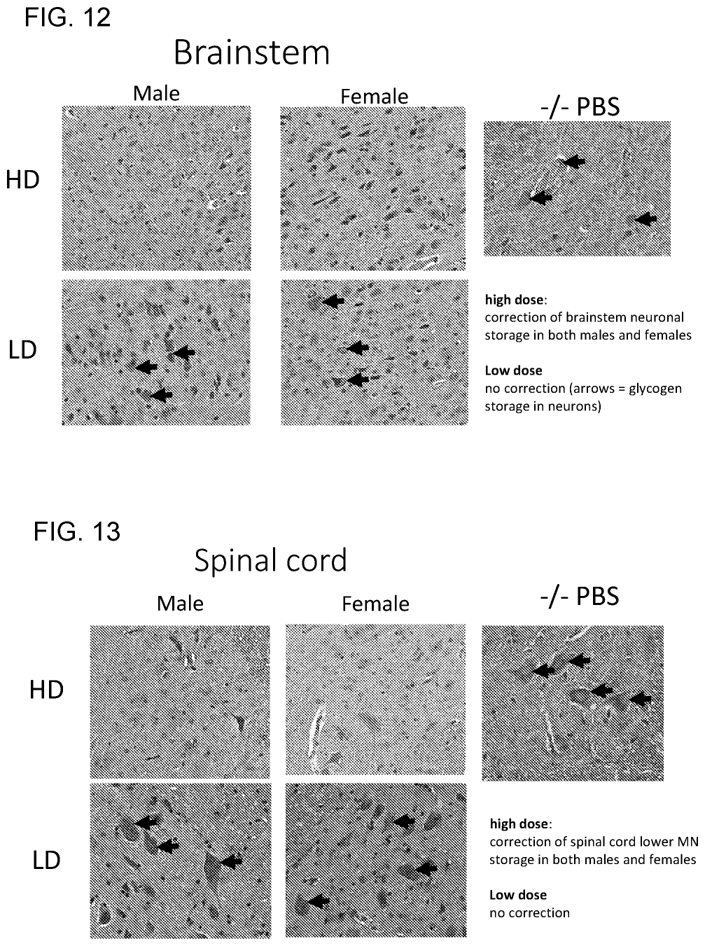

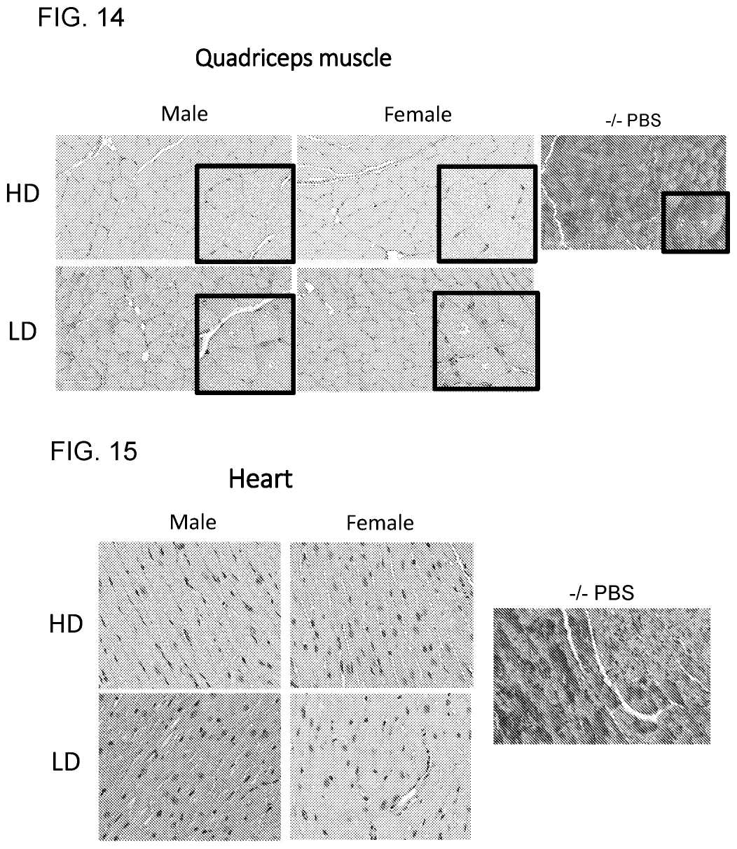

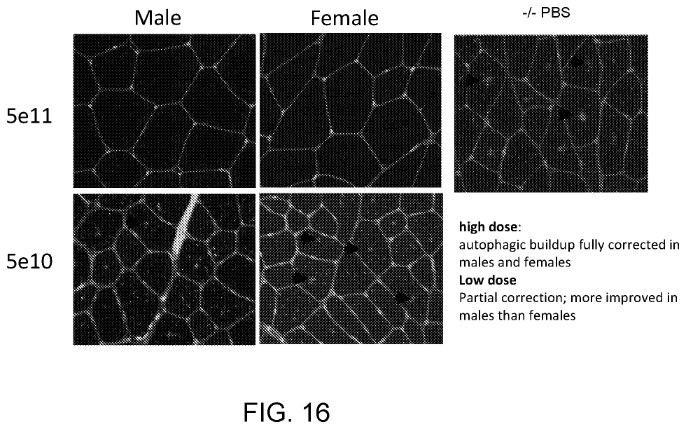

The effect of introducing miR183 target sites into the BiP-vIGF2-hGAAcoV780I vector genome was evaluated following IV delivery of AAVhu68 to Pompe mice. As was observed with the BiP-vIGF2.hGAAcoV780I construct (without miR183 targets), glycogen storage was corrected in the CNS after high dose intravenous administration of the vector including mir183 target sequences (FIG. 12 and FIG. 13). Glycogen storage and autophagic buildup in quadriceps were fully corrected after high dose intravenous administration, while glycogen storage correction and a partial correction of autophagic buildup were observed following low dose administration (FIG. 14). Correction of glycogen storage was also observed in the heart with both low and high doses (FIG. 15). Similar to what was observed with administration of CAG.BiP-vIGF2.hGAAcoV780I, autophagic buildup was fully resolved at high dose and markedly decreased at low dose (FIG. 16). The results confirmed that the addition of miR183 targets did not modify the efficacy of the therapeutic transgene compared to the corresponding vector lacking the miR target sequences.

### Example 4: Route of Administration and Dose Studies in Post-Symptomatic Aged Pompe Mice

The effects of route of administration and dose were evaluated in Pompe mice (as well as wildtype and vehicle controls) administered hGAA-encoding AAVhu68 vectors (including, e.g., AAVhu68.CAG.BiP-vIGF2.hGAAcoV780I.rBG) intravenously (IV) and/or via intracerebroventricular (ICV) injection. A dual-route of administration approach (intravenous and injection into the cerebrospinal fluid) using the same vector should correct both peripheral and neurological manifestations of the disease. Because a significant proportion of patients that will be eligible for gene therapy will already have advanced pathology, we elected to treat post-symptomatic Pompe mice (seven months of age) and to follow them for at least six months post treatment. Mice received two dose levels (low dose or high dose) of vector using either intravenous (IV), intracerebroventricular (ICV), or dual routes of administration. The doses used in this study (1×10¹¹ or 5×10¹⁰ GC ICV and 1×10¹³ GC/kg or 5×10¹³ GC/kg IV) correspond to the low and high doses used in the NHP study described in Example 6 and doses suitable for administration to humans (1×10¹³ GC/kg and 5×10¹³ GC/kg).

During the course of the study, mice were tested for locomotor activity using rotarod, wirehang, and grip strength evaluations, and plethysmography was performed. hGAA protein expression/activity and glycogen storage was measured in various tissues collected from treated mice, including plasma, quadricep muscle, gastrocnemius, diaphragm, and brain. Histology was performed to evaluate, for example, PAS (via Luxol fast blue staining), hGAA expression, and neuroinflammation (astrocytosis). Tissue sections were stained to evaluate autophagic buildup or clearance (for example, using antibodies that label LC3B).

A study design is provided in the table below.

| | | | | |
|---|---|---|---|---|
| −7 | | Day | | 90 |
| Baseline | | 30 | 60 | Blood |
| Blood collection | | Blood | Blood | Rotarod |
| Rotarod | C | Rotarod | Rotarod | Wirehang |
| WireHang | Vector | Wirehang | Wirehang | Grip Strength |
| Grip strength | dosing | Grip strength | Grip strength | Plethysinography |

| Group +190 | N | Geno. | ROA/Dose |
|---|---|---|---|
| 1 | 4M/4F | WT | ICV PBS |
| 2 | 4M/4F | KO | ICV HD (1e11 GC) |
| 3 | 4M/4F | KO | ICV LD (Se10 GC) |
| 4 | 4M/4F | KO | IV LD (1e13 GC/kg) |
| S | 4M/4F | KO | IV HD (5e13 GC/kg) |
| 6 | 4M/4F | KO | ICV LD + IV ID |
| 7 | 4M/4F | KO | ICV HD + IV HD |

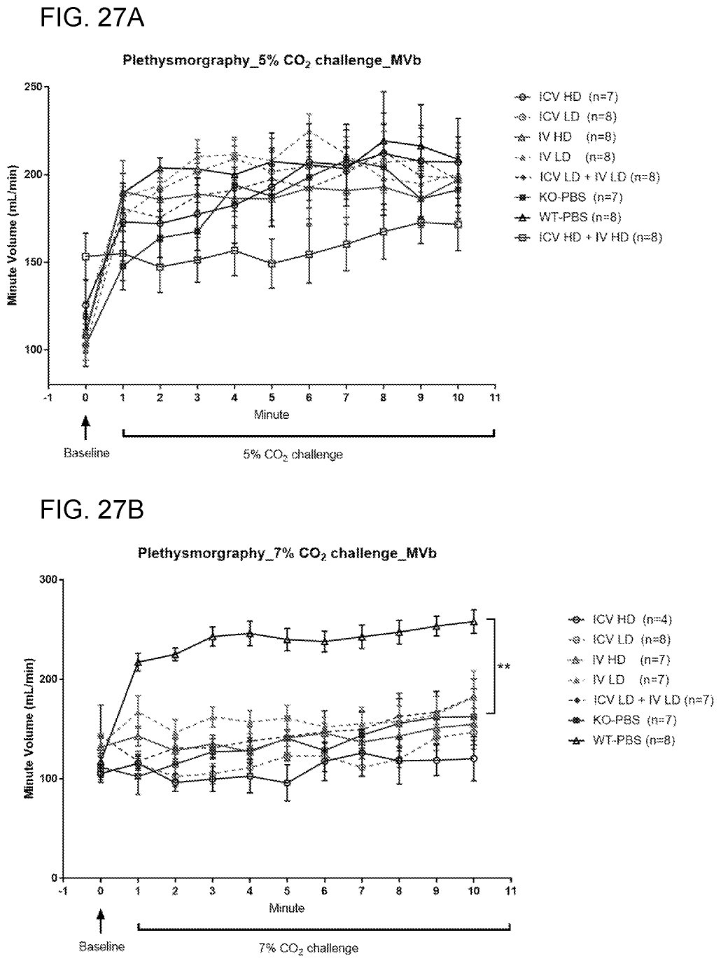

The results indicate that respiratory function, assessed by whole body plethysmography, was significantly ameliorated by treatment in mice receiving central nervous system-directed (ICV) vector. Respiratory function impairment in Pompe mice (and patients) is believed to be directly related to storage lesions in the motor neurons that innervate respiratory muscles. Improvement in respiratory function was observed in high-dose ICV treated Pompe mice, but not in IV-treated mice (FIG. 27A and FIG. 27B).

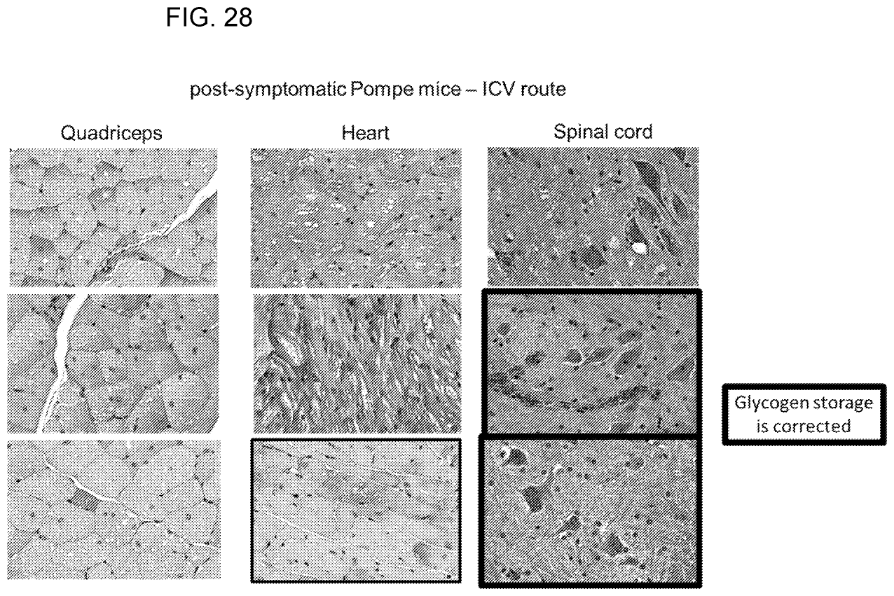

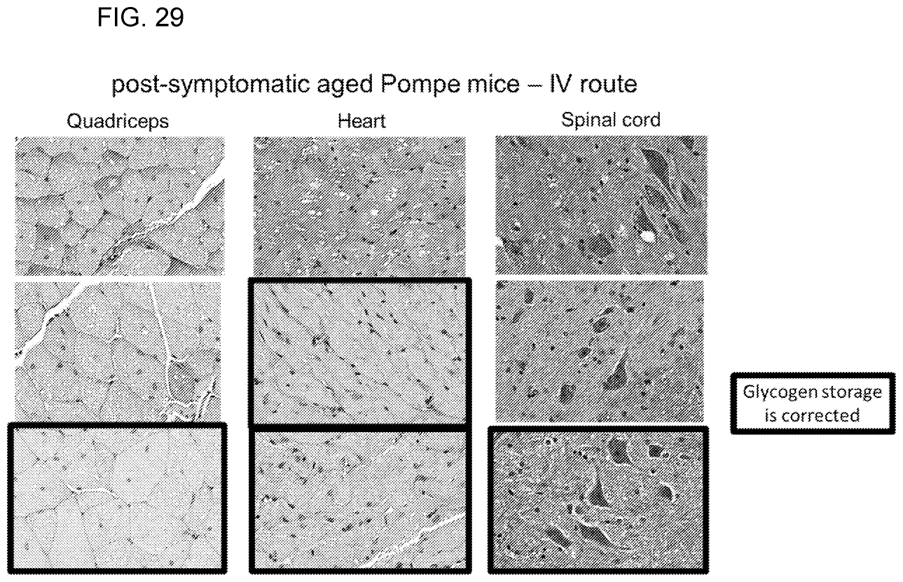

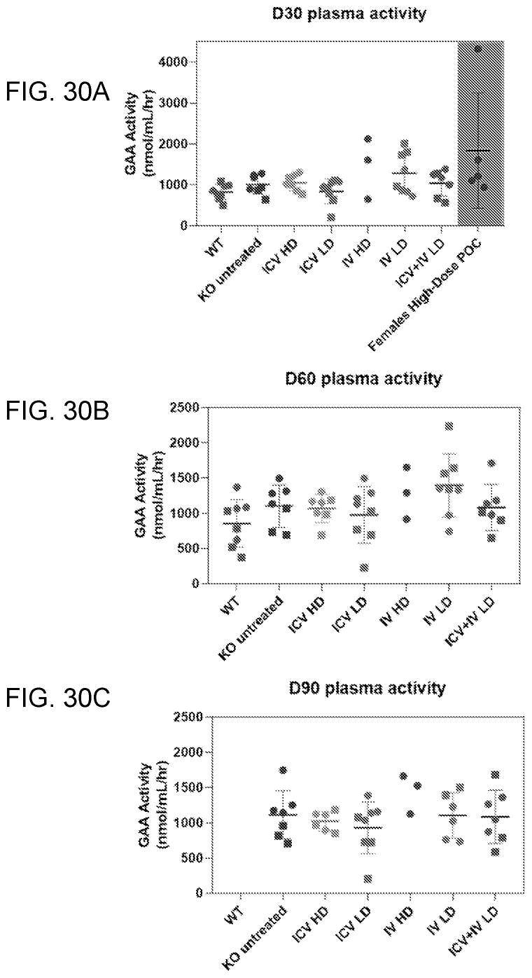

Histological studies were performed on quadriceps muscle, heart, and spinal cord samples from high dose and low dose ICV treated (FIG. 28) and high dose and low dose IV treated (FIG. 29) mice. Glycogen storage was corrected in spinal cord of mice that received a low or high vector dose via the ICV route. High dose IV administration was effective to correct glycogen storage in quadriceps muscle, heart, and spinal cord.

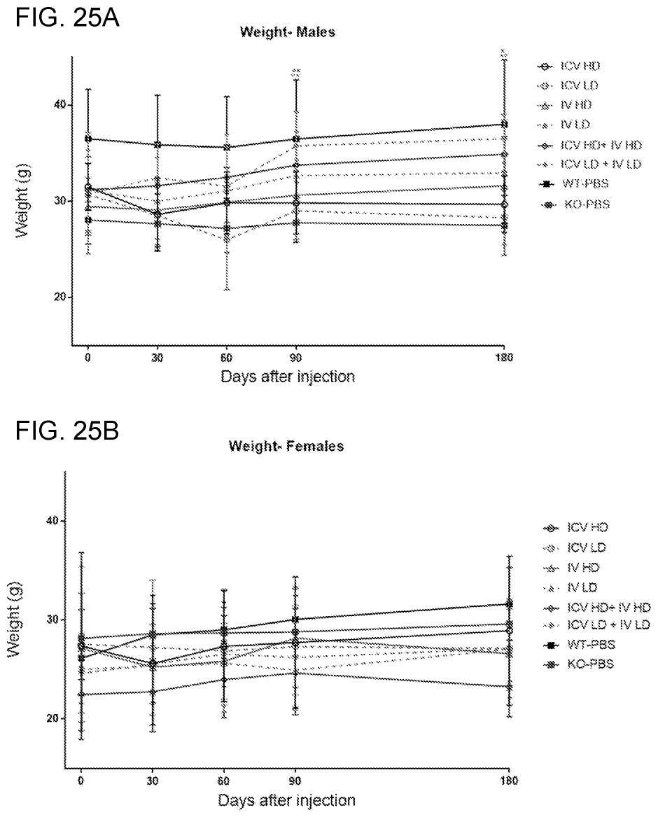

Body weight was significantly corrected in males treated with combinations of ICV and IV vectors (dual routes of administration) at both low doses and high doses (FIG. 25A). Single routes (IV alone or ICV alone) did not significantly correct body weights. Body weights did not differ between female Pompe and WT mice (FIG. 25B).

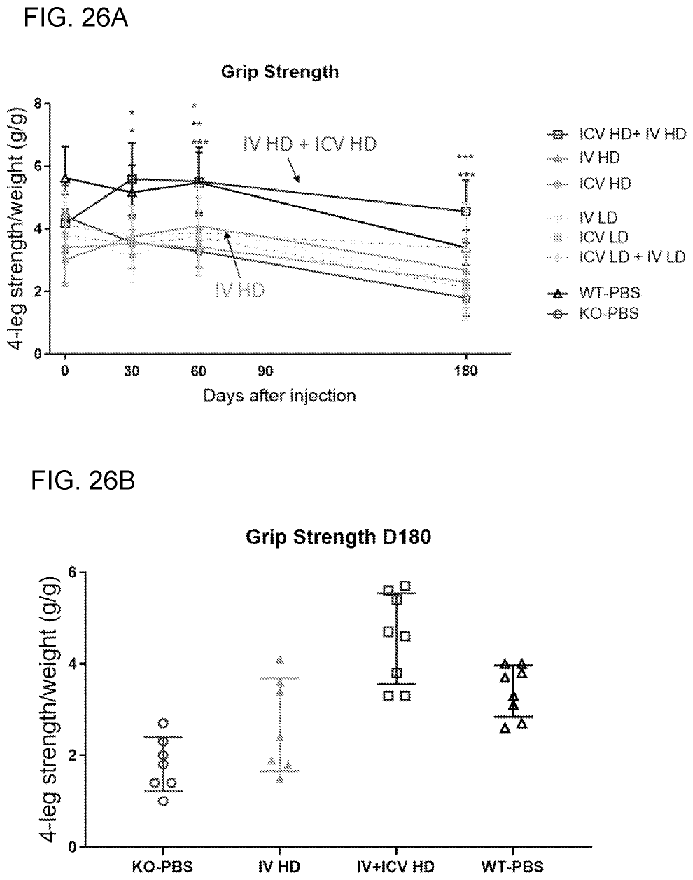

Grip strength was significantly improved for mice that received a high dose IV (compared to baseline and compared to PBS controls) (FIG. 26A). There was no significant benefit for low doses of vector administered ICV and IV or dual route administration (ICV LD+IV LC). However, administration of a combination of high doses IV and ICV rescued strength to wildtype levels as early as day 30 post injection and there was an incremental benefit of the combination at day 180 (FIG. 26B).

The findings support that a dual route of administration is preferable to target all aspects of the disease.

### Example 5: Administration of a DRG-Detargeting Gene Therapy Vector to Non-Human Primates

NHP primate studies were conducted to assess toxicity and to evaluate ICM delivery of CAG.BiP-IGF2-hGAAcoV780I or CAG.BiP-IGF2-hGAAcoV780I-4xmir183 in AAVhu68 capsids. The vectors were injected ICM at 3×10¹³ GC/kg and animals were sacrificed at day 35.

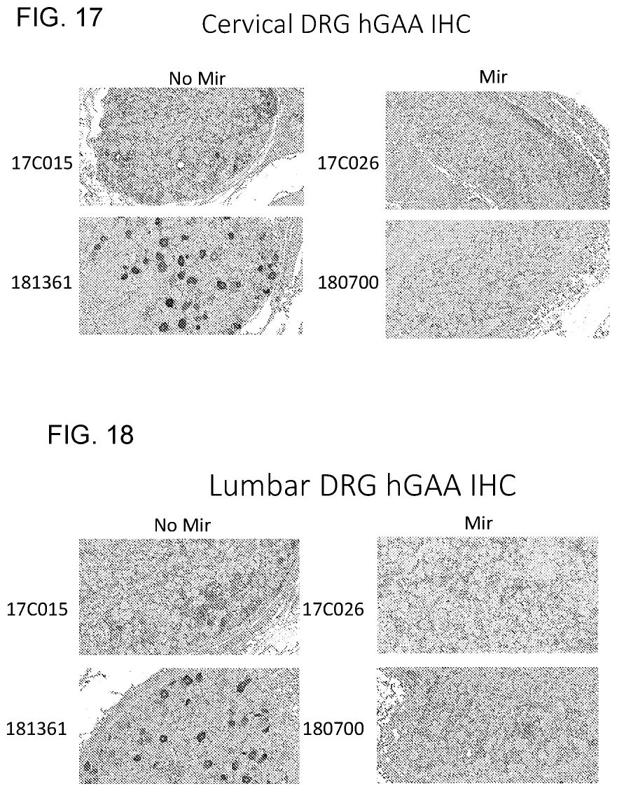

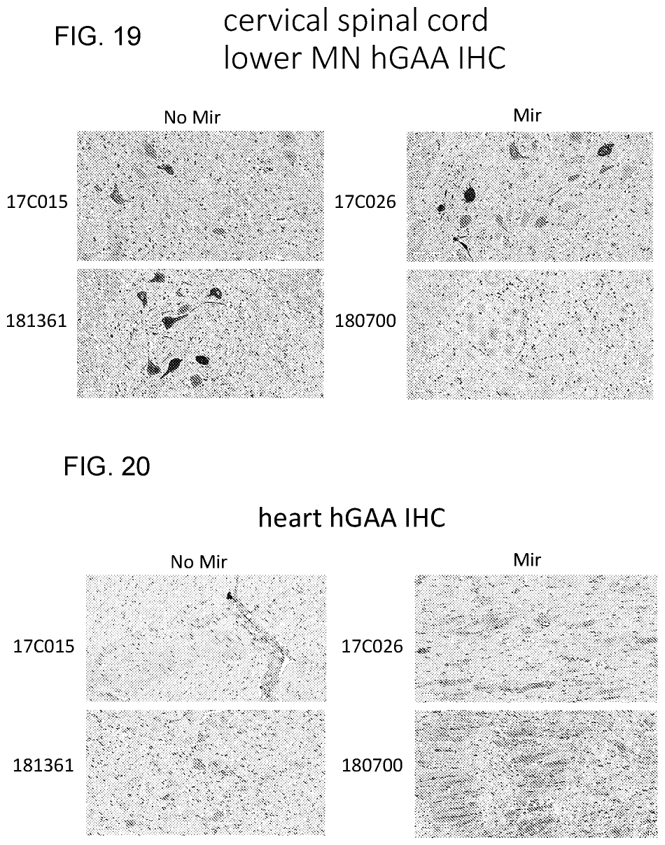

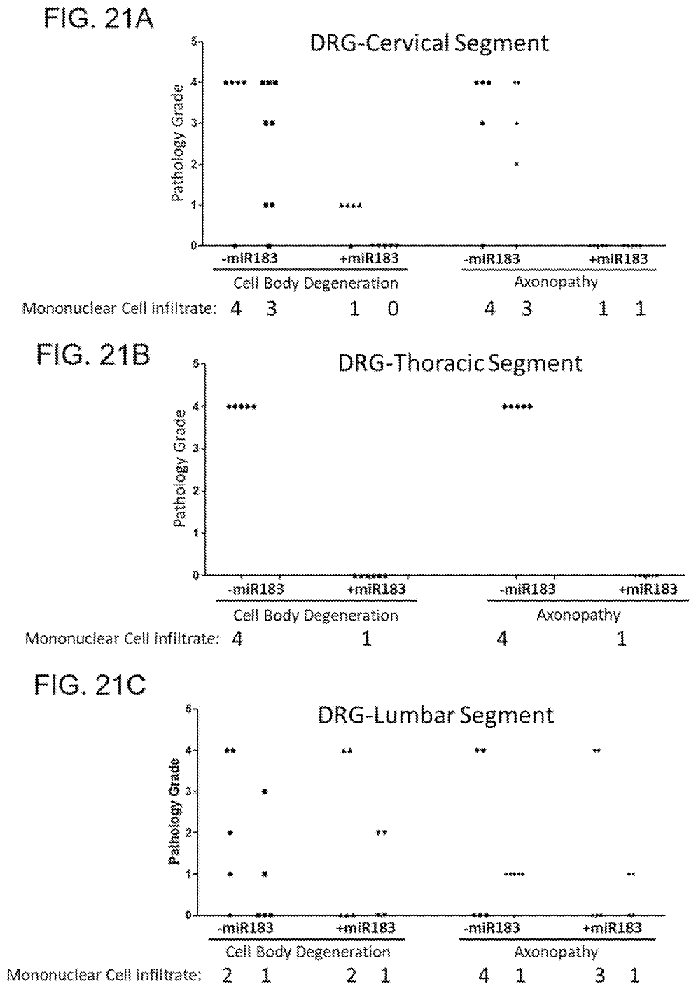

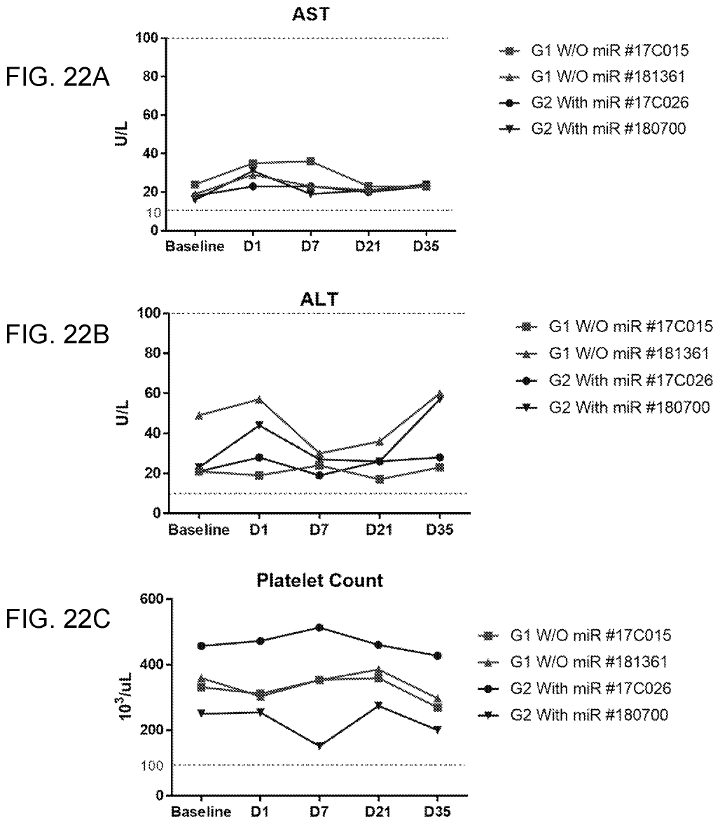

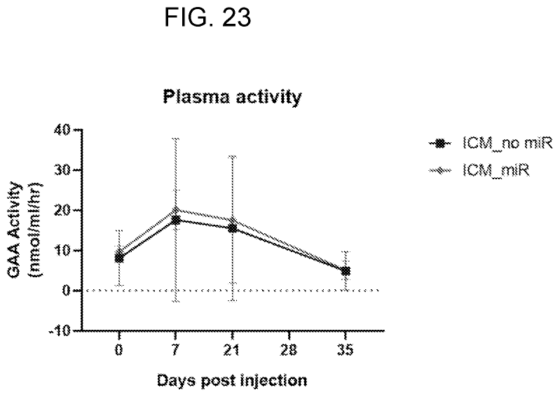

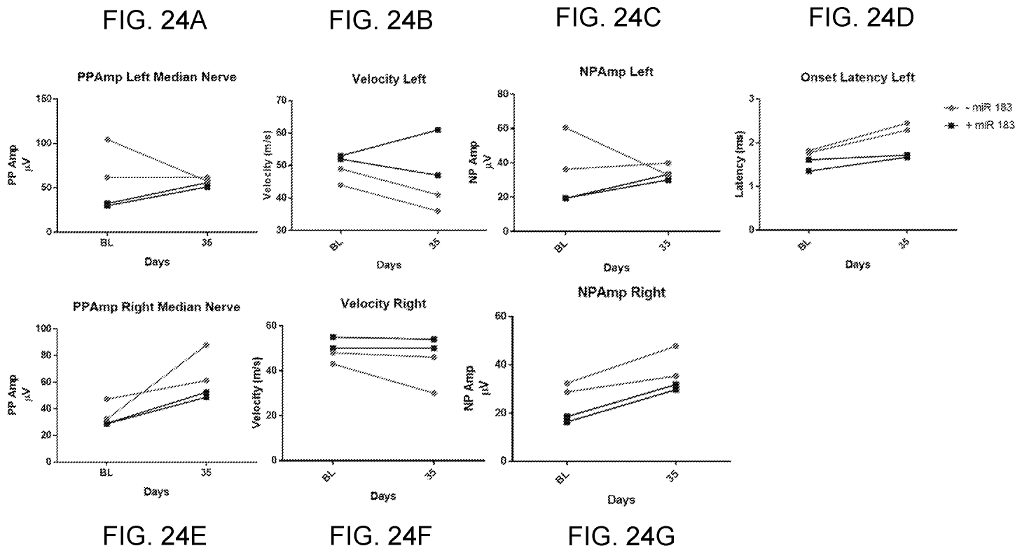

The addition of four tandem repeats of miR183 suppressed expression of the hGAA transgene in sensory neurons of the cervical DRG (FIG. 17). Markedly reduced expression of the hGAA transgene was also observed in sensory neurons of the lumbar DRG for the mir183 vector, but some expression remained (FIG. 18). Surprisingly, the presence of miR183 did not modify expression of the transgene in motor neurons (FIG. 19), which suggests that administration of the vector will be beneficial to reduce glycogen storage in the motor neurons of Pompe disease patients. In addition, there was no reduction in transgene expression in the heart following delivery of the miR183-containing construct (FIG. 20). In fact, there appeared to be increased expression in the heart, suggesting efficacy will be enhanced for cardiac disease treatment in Pompe disease patients. Notably, the tandem repeats of miR183 reduced toxicity in sensory neurons of the DRG from cervical and thoracic segments (FIG. 21A and FIG. 21B). There was no reduction in toxicity in the lumbar segment at this dose level (FIG. 21C), which is likely due to residual protein expression at the lumbar level as depicted in FIG. 18.

### Example 6: Route of Administration Studies in Non-Human Primates

NHP primate studies are conducted to assess toxicity and to evaluate alternative or combined routes of vector administration. For example, AAVhu68.CAG.BiP-IGF2-hGAAcoV780I or AAVhu68.CAG.BiP-IGF2-hGAAcoV780I-4xmir183 is injected IV at 5×10¹³ GC/kg (high dose) or 1×10¹³ GC/kg (low dose) or ICM at 3×10¹³ GC (high dose) or 1×10¹³ GC (low dose). The feasibility and toxicity of dual routes of administration is evaluated, for example, by administering the indicated IV high dose and ICM high dose or the IV low dose and ICM low dose. The combination of IV low dose and ICM low dose can reveal synergistic effects that will be beneficial in the treatment of Pompe patients.

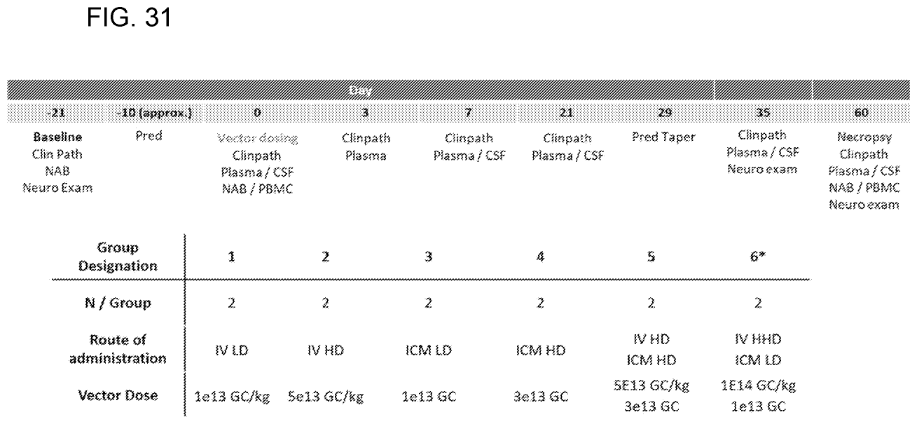

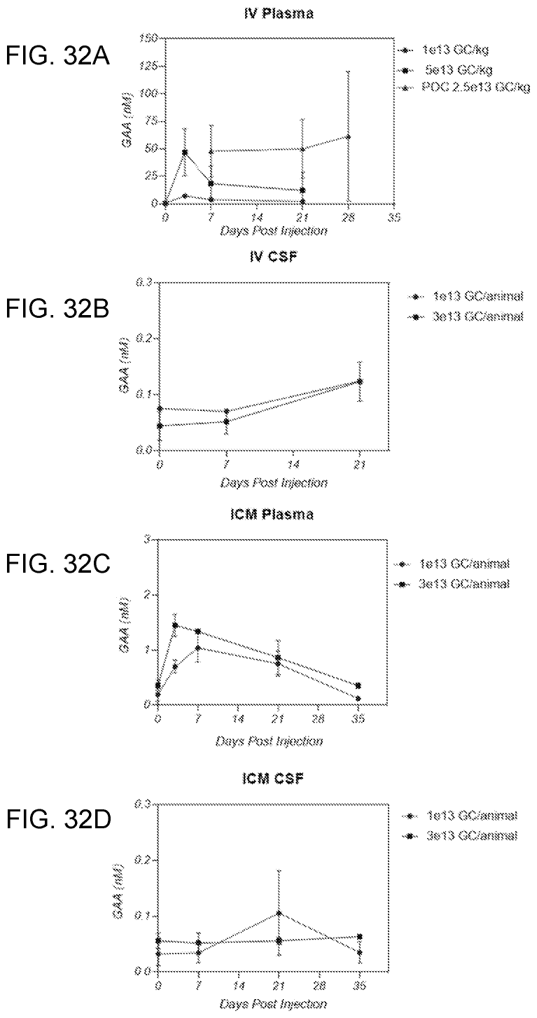

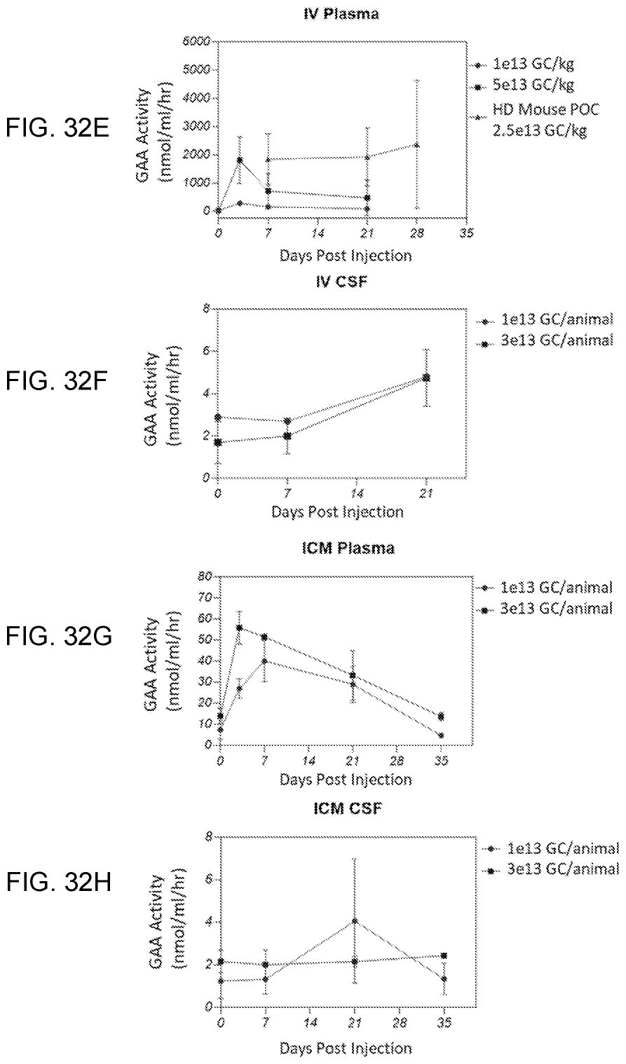

Throughout the study various readouts are used to detect hGAA signature peptide (plasma and CSF), to evaluate hGAA enzyme activity (serum and target tissues), and to measure anti-hGAA antibody titers (blood and CSF). Hisotopathology is performed to evaluate target tissues for hGAA expression and toxicity (e.g., H&E staining of CNS, heart, and muscle). A study design showing routes of administration and dosages is provided in FIG. 31.

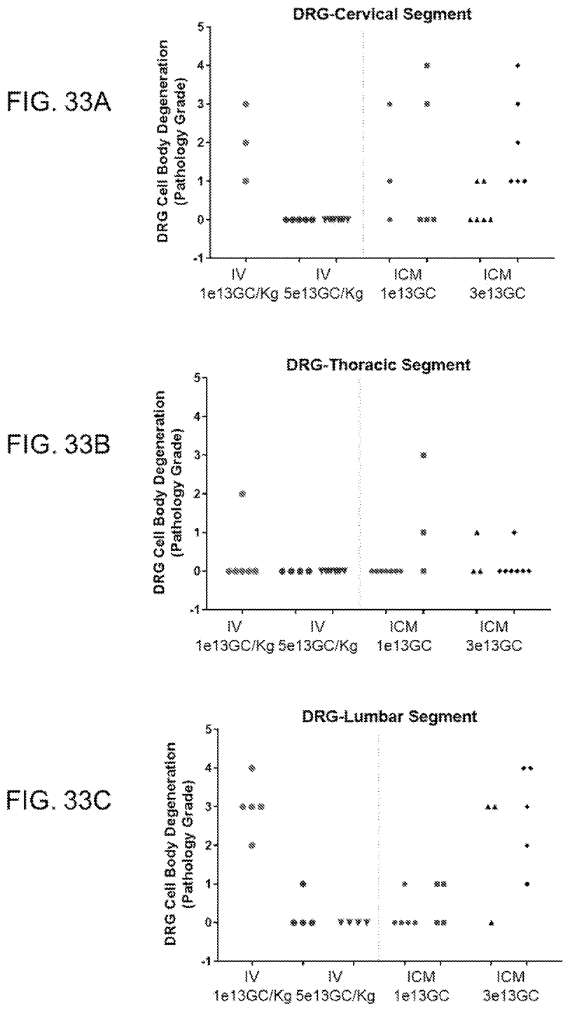

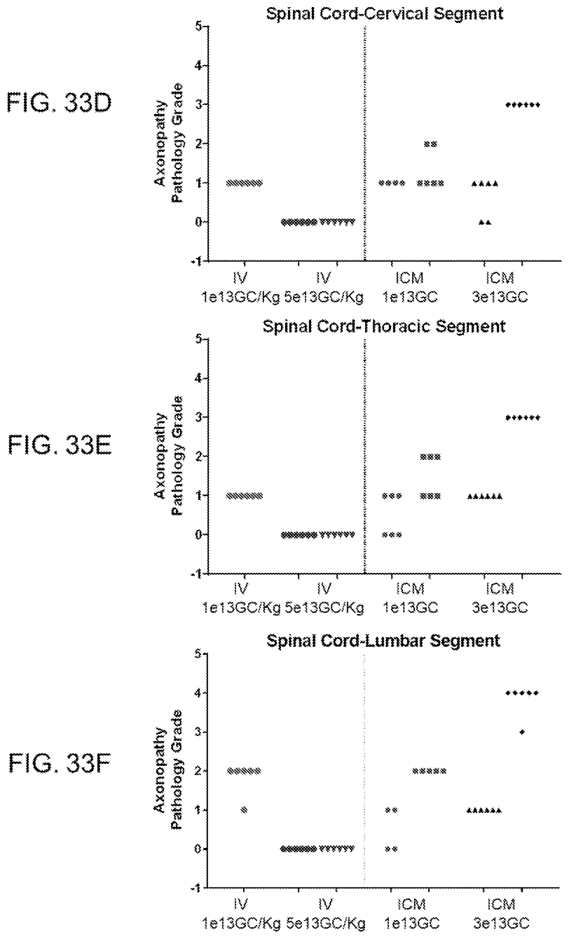

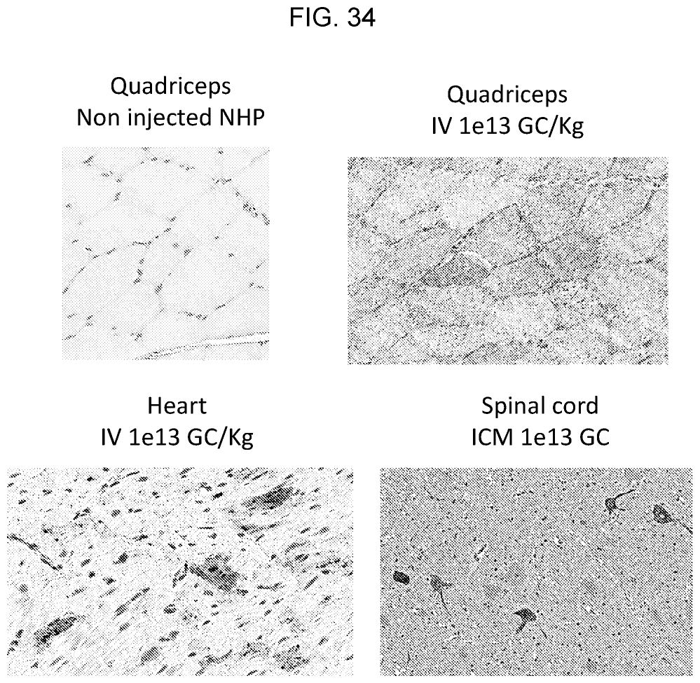

Preliminary studies evaluating single routes of administration revealed that low dose IV injected animals had expression of hGAA in quadriceps and heart (FIG. 34). IV injected animals also exhibited lower grades of spinal cord axonopathy than ICM injected animals (FIG. 33D-FIG. 33F). Expression of hGAA also observed by histology in the spinal cord of low dose ICM injected animals (FIG. 34). DRG degeneration and spinal cord axonopathy in ICM injected animals was not dose-dependent (FIG. 33A-FIG. 33F). In addition, one IV low dose animal (RA3607: 1e13 GC/Kg) had higher DRG degeneration, spinal cord axonopathy, and higher heart inflammatory responses than the IV high dose-injected animals.

(Sequence Listing Free Text)

| SEQ ID NO: | |
|---|---|
| (containing | |
| free text) | Free text under <223> |
| 3 | <223> synthetic construct |
| | <220> |
| | <221> MISC_FEATURE |
| | <222> (1)..(27) |
| | <223> Signal peptide |
| | <220> |
| | <221> MISC_FEATURE |
| | <222> (70)..(952) |
| | <220> |
| | <221> MISC_FEATURE |
| | <222> (123)..(952) |
| | <223> 76 kD GAA Protein with V780I |
| | <220> |
| | <221> MISC_FEATURE |
| | <222> (204)..(952) |
| | <223> 70 kD GAA Protein with V780I |
| 4 | <223> Engineered hGAAI Coding sequence |
| 6 | <223> Fusion Protein comprising |
| | hGAA780I |
| 7 | <223> Engineered sequence encoding |
| | fusion protein comprising GAAV780I |
| | <220> |
| | <221> misc_feature |
| | <222> (810)..(810) |
| | <223> V810I |
| 8 | <223> CAG promoter |
| | <220> |
| | <221> misc_feature |
| | <222> (1)..(243) |
| | <223> CMV early enhancer element |
| | <220> |
| | <221> misc_feature |
| | <222> (244)..(525) |
| | <223> Chicken Beta actin promoter |
| | <220> |
| | <221> misc_feature |
| | <222> (526)..(934) |
| | <223> hybrid intron |
| 9 | <223> Rabbit globin polyA |
| 12 | <223> Engineered hGAAV780I signal |
| | peptide |
| | <220> |
| | <221> sig_peptide |
| | <222> (1)..(81) |
| | <220> |
| | <221> CDS |
| | <222> (1)..(81) |
| 13 | <223> Synthetic Construct |
| 14 | <223> engineered hGAAV780I mature |
| | protein |
| | <220> |
| | <221> CDS |
| | <222> (1)..(2649) |
| 15 | <223> Synthetic Construct |
| 16 | <223> Engineered DNA for hGAA780I |
| | 123-890 |
| | <220> |
| | <221> CDS |
| | <222> (1)..(2304) |
| 17 | <223> Synthetic Construct |
| 18 | <223> Engineered hGAA 70 kD cDNA |
| | <220> |
| | <221> CDS |
| | <222> (1)..(2247) |
| 19 | <223> Synthetic Construct |
| 20 | <223> Engineered DNA for hGAAV780I 76 |
| | kD protein |
| | <220> |
| | <221> CDS |
| | <222> (1)..(2490) |
| 21 | <223> Synthetic Construct |
| 22 | <223> synthetic construct |
| | <220> |
| | <221> CDS |
| | <222> (1)..(2952) |
| | <220> |
| | <221> misc_feature |
| | <222> (1)..(270) |
| | <223> BiP signal peptide + vIGF2 + 2GS |
| | extension |
| | <220> |
| | <221> misc_feature |
| | <222> (271)..(2952) |
| | <223> engineered DNA for hGAA 61-952 |
| | 780I |
| | <220> |
| | <221> misc_feature |
| | <222> (2428)..(2430) |
| | <223> Ile codon |
| 23 | <223> Synthetic Construct |
| 24 | <223> synthetic construct |
| | <220> |
| | <221> CDS |
| | <222> (1)..(2952) |
| | <220> |
| | <221> misc_feature |
| | <222> (1)..(270) |
| | <223> BiP-vIGF peptide |
| | <220> |
| | <221> misc_feature |
| | <222> (1)..(270) |
| | <223> BiP signal peptide + vIGF2 + 2GS |
| | extension |
| | <220> |
| | <221> misc_feature |
| | <222> (271)..(2952) |
| | <223> hGAA 61-952 V780 DNA |
| | <220> |
| | <221> misc_feature |
| | <222> (2428)..(2430) |
| | <223> codon for hGAA 780 Valine |
| 25 | <223> Synthetic Construct |
| 26 | <223> miRNA target sequence |
| 27 | <223> miRNA target sequence |
| 28 | <223> synthetic construct |
| | <220> |
| | <221> misc_feature |
| | <222> (1)..(130) |
| | <223> 5′ ITR |
| | <220> |
| | <221> enhancer |
| | <222> (195)..(437) |
| | <223> CMV IE Enhancer |
| | <220> |
| | <221> promoter |
| | <222> (440)..(721) |
| | <223> chicken beta-actin promoter |
| | <220> |
| | <221> Intron |
| | <222> (721)..(1128) |
| | <223> hybrid intron in CAG |
| | <220> |
| | <221> CDS |
| | <222> (1141)..(4092) |
| | <223> BiP-vIGF2-hGAAco |
| | <220> |
| | <221> misc_feature |
| | <222> (3568)..(3570) |
| | <223> Ile codon |
| | <220> |
| | <221> polyA_signal |
| | <222> (4161)..(4287) |
| | <223> rabbit beta-globin poly a |
| | <220> |
| | <221> misc_feature |
| | <222> (4452)..(4581) |
| | <223> 3′ ITR |
| 29 | <223> Synthetic Construct |
| 30 | <223> synthetic construct |
| | <220> |
| | <221> misc_feature |
| | <222> (1)..(130) |
| | <223> 5′ ITR |
| | <220> |
| | <221> enhancer |
| | <222> (195)..(437) |
| | <223> CMV IE Enhancer |
| | <220> |
| | <221> promoter |
| | <222> (440)..(721) |
| | <223> chicken beta-actin promoter |
| | <220> |
| | <221> Intron |
| | <222> (721)..(1128) |
| | <223> Hybrid intron in CAG |
| | <220> |
| | <221> CDS |
| | <222> (1141)..(4092) |
| | <223> BiP-vIGF2-hGAAco |
| | <220> |
| | <221> misc_feature |
| | <222> (3568)..(3570) |
| | <223> Ile codon |
| | <220> |
| | <221> misc_feature |
| | <222> (4113)..(4134) |
| | <223> miR-183 target |
| | <220> |
| | <221> misc_feature |
| | <222> (4139)..(4160) |
| | <223> miR-183 target |
| | <220> |
| | <221> misc_feature |
| | <222> (4167)..(4188) |
| | <223> miR-183 target |
| | <220> |
| | <221> misc_feature |
| | <222> (4195)..(4216) |
| | <223> miR-183 target |
| | <220> |
| | <221> polyA_signal |
| | <222> (4267)..(4393) |
| | <223> rabbit beta-globin poly a |
| | <220> |
| | <221> misc_feature |
| | <222> (4558)..(4687) |
| | <223> 3′ ITR |
| 31 | <223> Synthetic Construct |
| 32 | <223> IGF2 F26S |
| 33 | <223> IGF2 Y27L |
| 35 | <223> V43L |
| 36 | <223> IGF2 F48T |
| 37 | <223> IGF2 R49S |
| 38 | <223> IGF2 S50I |
| 39 | <223> IGF2 A54R |
| 40 | <223> IGF2 L55R |
| 41 | <223> IGF2 F26S, Y27L, V43L, F48T, |
| | R49S, S50I, A54R, L55 |
| 42 | <223> IGF2 delta1-6, Y27L, K65R |
| 43 | <223> IGF2 delta1-7, Y27L, K65R |
| 44 | <223> IGF2 delta1-4, E6R, Y27L, K65R |
| 45 | <223> IGF2 delta1-4, E6R, Y27L |
| 46 | <223> IGF2 E6R |
| 48 | <223> vIGF2 delta1-4, E6R, Y27L, K65R |
| 20 | <223> Modified BiP-1 |
| 51 | <223> Modified BiP-2 |
| 52 | <223> Modified BiP-3 |
| 53 | <223> Modified BiP-4 |
| 55 | <223> linker sequence |
| 57 | <223> linker sequence |
| 58 | <223> linker sequence |
| 59 | <223> linker sequence |
| 60 | <223> linker sequence |

The following information is provided for sequences containing free text under numeric identifier <223>.

All documents cited in this specification are incorporated herein by reference. U.S. Provisional Patent Application No. 62/913,401, filed Oct. 10, 2019, and U.S. Provisional Patent Application No. 62/840,911, filed Apr. 30, 2019, are incorporated by reference in their entireties, together with their sequence listings. The sequence listing filed herewith named “19-8856PCT_ST25.txt” and the sequences and text therein are incorporated by reference. While the invention has been described with reference to particular embodiments, it will be appreciated that modifications can be made without departing from the spirit of the invention. Such modifications are intended to fall within the scope of the appended claims.

1. An expression cassette comprising a nucleic acid sequence encoding a chimeric fusion protein comprising a signal peptide and a vIGF2 peptide fused to a human acid-α-glucosidase (hGAA) comprising at least the active site of hGAA780I under the control of a regulatory sequences which direct its expression, wherein position 780 is based on the numbering of the positions of the amino acid sequence in SEQ ID NO: 3.

2. The expression cassette according to claim 1, wherein
(a) the hGAA comprises at least amino acids 204 to amino acids 890 of SEQ ID NO: 3 (hGAA780I), or a sequence at least 95% identical thereto which has an Ile at position 780;
(b) the hGAA comprises at least amino acids 204 to amino acids 952 of SEQ ID NO: 3, or a sequence at least 95% identical thereto which has an Ile at position 780;
(c) the hGAA comprises at least amino acids 123 to amino acids 890 of SEQ ID NO: 3, or a sequence at least 95% identical thereto which has an Ile at position 780;
(d) the hGAA comprises at least amino acids 70 to amino acids 952 of SEQ ID NO: 3, or a sequence at least 95% identical thereto which has an Ile at position 780; or
(e) the hGAA comprises at least amino acids 70 to amino acids 890 of SEQ ID NO: 3, or a sequence at least 95% identical thereto which has an Ile at position 780.

3-6. (canceled)

7. The expression cassette according to any one of claims 1 to 6, wherein the hGAA780I is encoded by SEQ ID NO: 4, or a sequence at least 95% identical thereto, or wherein the hGAA780I is encoded by SEQ ID NO: 5, or a sequence at least 95% identical thereto.

8. (canceled)

9. The expression cassette according to any claim 1, wherein the fusion protein comprises SEQ ID NO: 6, or a sequence at least 95% identical thereto and/or wherein the fusion protein is encoded by SEQ ID NO: 7, or a sequence at least 95% identical thereto.

10. (canceled)

11. The expression cassette according to claim 1, further comprising at least two tandem repeats of miR target sequences, wherein the at least two tandem repeats comprise at least a first miRNA target sequence and at least a second miRNA target sequence which may be the same or different and are operably linked 3′ to the sequence encoding the fusion protein.

12-13. (canceled)

14. The expression cassette according to claim 1, wherein the vIGF2 peptide comprises an amino acid sequence that is at least 90% identical to SEQ ID NO: 32 and having at least one substitution at one or more positions selected from positions 6, 26, 27, 43, 48, 49, 50, 54, 55, and 65 of SEQ ID NO: 32.

15-17. (canceled)

18. The expression cassette according to claim 1, wherein the vIGF2 peptide comprises an N-terminal deletion at position 1 of SEQ ID NO: 32 or positions 1 to 4 of SEQ ID NO: 32.

19-21. (canceled)

22. The expression cassette according to claim 1, wherein the nucleic acid sequence further comprises a linker sequence encoding a linker peptide between the vIGF2 nucleotide sequence and the nucleic acid sequence encoding hGAA780I.

23. (canceled)

24. The expression cassette according to claim 1, wherein the signal peptide is a binding immunoglobulin protein (BiP) signal peptide or a Gaussia signal peptide,
wherein the BiP signal peptide comprises an amino acid sequence at least 90% identical to any one of SEQ ID NOs: 49-53, and
wherein the Gaussia signal peptide comprises an amino acid sequence at least 90% identical to SEQ ID NO: 54.

25-29. (canceled)

30. The expression cassette according to claim 1, wherein the expression cassette is carried by a viral vector selected from a recombinant parvovirus, a recombinant lentivirus, a recombinant retrovirus, and a recombinant adenovirus.

31-32. (canceled)

33. The expression cassette according to claim 1, wherein the expression cassette is carried by a non-viral vector selected from naked DNA, naked RNA, an inorganic particle, a lipid particle, a polymer-based vector, or a chitosan-based formulation.

34. A recombinant adeno-associated virus (rAAV) comprising:
(a) an AAV capsid which targets cells of at least one of muscle, heart, and the central nervous system; and
(b) a vector genome packaged in the AAV capsid, said vector genome comprising a nucleic acid sequence encoding a chimeric fusion protein comprising a signal peptide and a vIGF2 peptide fused to a hGAA comprising at least the active site of hGAA780I under control of a regulatory sequences which direct its expression, wherein position 780 is based on the numbering of the positions of the amino acid sequence in SEQ ID NO: 3.

35. The rAAV according to claim 34, wherein
(a) the hGAA comprises at least amino acids 204 to amino acids 890 of SEQ ID NO: 3 (hGAA780I), or a sequence at least 95% identical thereto which has an Ile at position 780;
(b) the hGAA comprises at least amino acids 204 to amino acids 952 of SEQ ID NO: 3, or a sequence at least 95% identical thereto which has an Ile at position 780;
(c) the hGAA comprises at least amino acids 123 to amino acids 890 of SEQ ID NO: 3, or a sequence at least 95% identical thereto which has an Ile at position 780;
(d) the hGAA comprises at least amino acids 70 to amino acids 952 of SEQ ID NO: 3, or a sequence at least 95% identical thereto which has an Ile at position 780; or
(e) the hGAA comprises at least amino acids 70 to amino acids 890 of SEQ ID NO: 3, or a sequence at least 95% identical thereto which has an Ile at position 780.

36-39. (canceled)

40. The rAAV according to claim 34, wherein the hGAA780I is encoded by SEQ ID NO: 4, or a sequence at least 95% identical thereto, or wherein the hGAA780I is encoded by SEQ ID NO: 5, or a sequence at least 95% identical thereto.

41. (canceled)

42. The rAAV according to claim 34, wherein the fusion protein comprises SEQ ID NO: 6, or a sequence at least 95% identical thereto and/or wherein the fusion protein is encoded by SEQ ID NO: 7, or a sequence at least 95% identical thereto.

43. (canceled)

44. The rAAV according to claim 34, wherein the vector genome further comprises at least two tandem repeats of dorsal root ganglion (DRG)-specific miR-183 target sequences, wherein the at least two tandem repeats comprise at least a first miRNA target sequence and at least a second miRNA target sequence which may be the same or different and are operably linked 3′ to the sequence encoding the fusion protein.

45-46. (canceled)

47. The rAAV according to claim 34, wherein the vIGF2 peptide comprises an amino acid sequence that is at least 90% identical to SEQ ID NO: 32 and having at least one substitution at one or more positions selected from positions 6, 26, 27, 43, 48, 49, 50, 54, 55, and 65 of SEQ ID NO: 32.

48-50. (canceled)

51. The rAAV according to claim 34, wherein the vIGF2 peptide comprises an N-terminal deletion at position 1 of SEQ ID NO: 32 or positions 1 to 4 of SEQ ID NO: 32.

52-54. (canceled)

55. The rAAV according to claim 34, wherein the nucleic acid sequence further comprises a linker sequence encoding a linker peptide between the vIGF2 nucleotide sequence and the nucleic acid sequence encoding hGAA780I.

56. (canceled)

57. The rAAV according to claim 34, wherein the signal peptide is selected from a binding immunoglobulin protein (BiP) signal peptide and a Gaussia signal peptide,
wherein the BiP signal peptide comprises an amino acid sequence at least 90% identical to any one of SEQ ID NOs: 49-53, and
wherein the Gaussia signal peptide comprises an amino acid sequence at least 90% identical to SEQ ID NO: 54.

58-62. (canceled)

63. The rAAV according to claim 34, wherein the vector genome comprises SEQ ID NO: 30, or a sequence at least 95% identical thereto.

64. The rAAV according to claim 34, wherein the capsid is a clade F capsid, optionally an AAVhu68 capsid.

65. (canceled)

66. A plasmid comprising a sequence encoding the expression cassette according to claim 1.

67-68. (canceled)

69. A host cell containing the plasmid according to claim 66.

70. (canceled)

71. A composition comprising the rAAV according to claim 3 and at least one of a pharmaceutically acceptable carrier, an excipient, and/or a suspending agent.

72-73. (canceled)

74. A method for treating a patient having Pompe disease comprising delivering to the patient the rAAV according to claim 34.

75-76. (canceled)

77. A method for improving cardiac, respiratory and/or skeletal muscle function in a patient having a deficiency in alpha-glucosidase (GAA), said method comprising delivering to the patient the rAAV according to claim 34.

78-83. (canceled)


SEQUENCE LISTING (64 sequences)

SEQ ID NO: 1 - DNA, 2211 bp, adeno-associated virus hu.68
  CDS (1)..(2211)
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctaacgcttaatgatggaagccaagccgtgggtcgttcgtccttttactgcctggaatatttcccgtcgcaaatgctaagaacgggtaacaacttccagttcagctacgagtttgagaacgtacctttccatagcagctatgctcacagccaaagcctggaccgactcatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccagtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtttattctgaaccccgccccattggcaccagatacctgactcgtaatctgtaa

SEQ ID NO: 2 - protein, 736 aa, adeno-associated virus hu.68
MAADGYLPDWLEDNLSEGIREWWALKPGAPQPKANQQHQDNARGLVLPGYKYLGPGNGLDKGEPVNEADAAALEHDKAYDQQLKAGDNPYLKYNHADAEFQERLKEDTSFGGNLGRAVFQAKKRLLEPLGLVEEAAKTAPGKKRPVEQSPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3 - protein, 952 aa, Artificial Sequence [sequence omitted]

SEQ ID NO: 4 - DNA, 2856 bp, Artificial sequence
  Engineered hGAAI Coding sequence
atgggcgttagacaccctccttgctctcacagactgctggccgtgtgtgctctggtgtctctggctacagctgccctgctgggacatatcctgctgcacgactttctgctggtgcccagagagctgtctggcagctctcctgtgctggaagagacacaccctgcacatcagcagggcgcctctagacctggacctagagacgctcaggcccatcctggcagacctagagctgtgcccacacagtgtgacgtgccacctaacagcagattcgactgcgcccctgacaaggccatcacacaagagcagtgtgaagccagaggctgctgctacatccctgccaaacaaggactgcagggcgctcagatgggacagccctggtgcttcttcccaccatcttaccccagctacaagctggaaaacctgagcagcagcgagatgggctacaccgccacactgaccagaaccacacctacattcttcccgaaggacatcctgacactgcggctggacgtgatgatggaaaccgagaaccggctgcacttcaccatcaaggaccccgccaatcggagatacgaggtgccactggaaacccctcacgtgcactctagagccccatctccactgtacagcgtggaattttccgaggaacccttcggcgtgatcgtgcggagacagctggatggaagagtgctgctgaacaccacagtggcccctctgttcttcgccgaccagtttctgcagctgagcaccagcctgcctagccagtatatcacaggcctggccgagcacctgtctccactgatgctgagcacatcctggaccagaatcaccctgtggaacagagatctggcccctacacctggcgccaacctgtacggctctcaccccttttatctggccctggaagacggcggatctgcccacggtgtttttctgctgaactccaacgccatggacgtggtgctgcagccatctcctgctctgtcttggagaagcacaggcggcatcctggacgtgtacatctttctgggccccgagcctaagagcgtggtgcagcagtatctggacgtcgtgggctaccccttcatgcctccttattggggcctgggcttccacctgtgcaggtggggatacagcagcaccgccatcaccagacaggtggtggaaaacatgacccgggctcacttcccactggacgtgcagtggaacgacctggactacatggacagcagacgggacttcaccttcaacaaggacggcttcagagacttccccgccatggtgcaagaactgcaccaaggcggcagacggtacatgatgattgtggacccagccatcagctctagcggccctgccggaagctacagaccttacgatgagggcctgagaagaggcgtgttcatcaccaacgagacaggccagcctctgatcggcaaagtgtggcctggcagcacagcctttccagacttcacaaaccccaccgctctggcttggtgggaagatatggtggccgagttccacgatcaggtgcccttcgatggcatgtggatcgacatgaacgagcccagcaacttcatccggggcagcgaggacggctgccccaacaacgaactggaaaatcctccttacgtgcccggcgttgtcggcggaacacttcaggccgctacaatctgtgccagcagccaccagttcctcagcacccactacaacctgcacaacctgtacggcctgaccgaggccattgcctctcatagagccctggttaaggccagaggcacccggccttttgtgatcagcagaagcacattcgccggccacggcagatacgccggacattggacaggcgacgtgtggtctagttgggagcagctggctagcagcgtgccagagatcctgcagttcaatctgctgggcgtgccactcgtgggagccgacgtttgtggcttcctgggcaacaccagcgaggaactgtgtgtgcgttggacacagctgggcgccttctatcccttcatgagaaaccacaacagcctgctgagcctgcctcaagagccctacagctttagcgagcctgcacagcaggccatgagaaaggccctgactctgagatacgctctgctgccccacctgtacaccctgtttcaccaggctcacgtggccggggagacagtggctagacctctgttcctggaatttcccaaggacagctccacctggaccgtggatcatcagctgctgtggggagaagccctgctcatcacacctgttctgcaggccggaaaggccgaagtgaccggctattttcctctcggcacttggtacgacctgcagaccgtgcctattgaggccctgggatctcttcctccacctcctgctgctcctagagagcctgccatccactctgaaggccagtgggttacactgcccgctcctctggacaccatcaacgtgcacctgagagctggctacatcatcccactgcaaggccctggcctgaccacaaccgaatctagacagcagcccatggctctggccgtggctcttacaaaaggcggagaggctagaggcgagctgttctgggacgacggcgagtctctggaagtgctggaacggggcgcttatacccaagtgatcttcctggccagaaacaacaccatcgtgaacgaactcgtgcgcgtgaccagtgaaggtgctggactgcaactgcagaaagtgaccgtgctcggagtggccacagctcctcaacaggtgctgtctaacggcgtgcccgtgtccaacttcacatacagccccgacaccaaggtcctggacatctgtgtgtcactgctgatgggcgagcagttcctggtgtcctggtgc

SEQ ID NO: 5 - DNA, 2859 bp, Homo sapiens
atgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagtagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagcttttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 6 - protein, 982 aa, Artificial sequence
  Fusion Protein comprising hGAA780I
MKLSLVAAMLLLLSAARASRTLCGGELVDTLQFVCGDRGFLFSRPASRVSRRSRGIVEECCFRSCDLALLETYCATPARSEGGGGSGGGGSRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 7 - DNA, 2952 bp, Artificial sequence
  Engineered sequence encoding fusion protein comprising GAAV780I
  misc_feature (810)..(810): V810I
atgaagctgtctctggtggctgctatgctgctgctcctgtctgccgccagagccagcagaacactttgtggcggagagctggtggacaccctgcagtttgtgtgtggcgacagaggcttcctgttcagcagacctgccagccgggtttccagacggtctagaggaatcgtggaagagtgctgcttcagaagctgcgatctggccctgctggaaacctactgtgccacaccagccagatctgaaggcggcggaggatctggcggaggcggatctagacctggacctagagacgcccaggctcaccctggtagacctagagctgtgcctacacagtgcgacgtgccacctaacagcagattcgactgcgcccctgacaaggccatcacacaagagcagtgtgaagccagaggctgctgctacatccctgccaaacaaggactgcagggcgcccagatgggacagccttggtgcttcttcccaccatcttaccccagctacaagctggaaaacctgagcagctccgagatgggctacaccgccacactgaccagaaccacacctacattcttcccgaaggacatcctgacactgcggctggacgtgatgatggaaaccgagaaccggctgcacttcaccatcaaggaccccgccaatcggagatacgaggtgcccctggaaacaccccacgtgcactctagagcaccctctccactgtacagcgtggaattttccgaggaacccttcggcgtgatcgtgcggagacagctggatggcagagtgctcctgaataccacagtggcccctctgttcttcgccgaccagtttctgcagctgagcaccagcctgcctagccagtatatcacaggcctggccgagcatctgagccctctgatgctgagcacatcctggaccagaatcaccctgtggaaccgcgacctggctcctacacctggcgccaatctgtacggctctcaccccttctacctggcactggaagacggtggatctgcccacggtgtctttctgctgaatagcaacgccatggacgtggtgctgcagccctctcctgcactgtcttggagatctacaggcggcatcctggacgtgtacatctttctgggccccgagcctaagagcgtggtgcagcagtatctggacgtcgtgggctaccccttcatgcctccttattggggcctgggcttccacctgtgtaggtggggctacagcagcaccgccatcaccagacaggtggtggaaaacatgacccgggctcacttcccactggacgtgcagtggaacgacctggactacatggacagcagacgggacttcaccttcaacaaggacggcttcagagacttccccgccatggtgcaagagctgcatcaaggcggacggcggtacatgatgattgtggaccctgccatcagcagctctggaccagccggcagctacagaccttacgatgagggactgagaagaggcgtgttcatcaccaacgagacaggccagcctctgatcggcaaagtgtggcctggcagcacagcctttccagacttcacaaaccccaccgctctggcttggtgggaagatatggtggccgagttccacgatcaggtgcccttcgatggcatgtggatcgacatgaacgagcccagcaacttcatccggggcagcgaggatggctgccccaacaacgaactagaaaatcctccttacgtgcccggcgttgtcggcggaacacttcaggccgctacaatctgtgccagcagccatcagtttctgagcacccactacaacctgcacaacctgtacggcctgaccgaggccattgcctctcatagagccctggttaaggccagaggcacccggccttttgtgatcagcagaagcacattcgccggccacggcagatacgcaggacattggacaggcgacgtgtggtctagttgggagcagctggctagcagcgtgccagagatcctgcagttcaatctgctgggcgtgccactcgtgggagccgacgtttgtggcttcctgggcaacaccagcgaggaactgtgtgtgcgttggacacagctgggcgccttctatcccttcatgagaaaccacaacagcctgctgagcctgcctcaagagccctacagctttagcgagcctgcacagcaggccatgagaaaggccctgactctgagatacgccctgctgcctcacctgtacaccctgtttcatcaggcccacgtggcaggcgagacagtggctagacctctgttcctggaatttcccaaggacagctccacctggaccgtggatcatcagctgctgtggggagaagccctgctgattacaccagtgctgcaggccggaaaggccgaagtgacaggctatttccctctcggcacttggtacgacctgcagaccgtgcctatcgaggctctgggatctcttcctccacctcctgccgctcctagagagcctgccattcactctgaaggccagtgggttaccctgcctgctcctctggacaccatcaacgtgcacctgagagccggctacatcatccctctgcaaggccctggcctgaccacaaccgaatctagacagcagcccatggcactggccgtggctcttacaaaaggcggagaggctagaggcgagctgttctgggatgatggcgagagcctagaagtgctggaacggggcgcttatacccaagtgatcttcctggccagaaacaacaccatcgtgaacgaactcgtgcgcgtgaccagtgaaggtgctggactgcaactgcagaaagtgaccgtgctcggagtggccacagctcctcagcaggttctgtctaatggcgtgcccgtgtccaacttcacatacagccccgacaccaaggtcctggacatctgtgtgtccctgcttatgggcgagcagttcctggtgtcctggtgctgataa

SEQ ID NO: 8 - DNA, 934 bp, Artificial sequence
  CAG promoter
  misc_feature (1)..(243): CMV early enhancer element
  misc_feature (244)..(525): Chicken Beta actin promoter
  misc_feature (526)..(934): hybrid intron in CAG (b-actin intron and rabbit beta 1-globin
  misc_feature (526)..(934): hybrid intron
attgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 9 - DNA, 127 bp, Artificial sequence
  Rabbit globin polyA
gatctttttccctctgccaaaaattatggggacatcatgaagccccttgagcatctgacttctggctaataaaggaaatttattttcattgcaatagtgtgttggaattttttgtgtctctcactcg

SEQ ID NO: 10 - DNA, 188 bp, adeno-associated virus 2
gctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaaga

SEQ ID NO: 11 - DNA, 188 bp, adeno-associated virus 2
tcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagc

SEQ ID NO: 12 - DNA, 81 bp, Artificial sequence
  Engineered hGAAV780I signal peptide
  sig_peptide (1)..(81)
  CDS (1)..(81)
atgggcgttagacaccctccttgctctcacagactgctggccgtgtgtgctctggtgtctctggctacagctgccctgctg

SEQ ID NO: 13 - protein, 27 aa, Artificial sequence
  Synthetic Construct
MGVRHPPCSHRLLAVCALVSLATAALL

SEQ ID NO: 14 - DNA, 2649 bp, Artificial Sequence
  engineered hGAAV780I mature protein
  CDS (1)..(2649)
gcccatcctggcagacctagagctgtgcccacacagtgtgacgtgccacctaacagcagattcgactgcgcccctgacaaggccatcacacaagagcagtgtgaagccagaggctgctgctacatccctgccaaacaaggactgcagggcgctcagatgggacagccctggtgcttcttcccaccatcttaccccagctacaagctggaaaacctgagcagcagcgagatgggctacaccgccacactgaccagaaccacacctacattcttcccgaaggacatcctgacactgcggctggacgtgatgatggaaaccgagaaccggctgcacttcaccatcaaggaccccgccaatcggagatacgaggtgccactggaaacccctcacgtgcactctagagccccatctccactgtacagcgtggaattttccgaggaacccttcggcgtgatcgtgcggagacagctggatggaagagtgctgctgaacaccacagtggcccctctgttcttcgccgaccagtttctgcagctgagcaccagcctgcctagccagtatatcacaggcctggccgagcacctgtctccactgatgctgagcacatcctggaccagaatcaccctgtggaacagagatctggcccctacacctggcgccaacctgtacggctctcaccccttttatctggccctggaagacggcggatctgcccacggtgtttttctgctgaactccaacgccatggacgtggtgctgcagccatctcctgctctgtcttggagaagcacaggcggcatcctggacgtgtacatctttctgggccccgagcctaagagcgtggtgcagcagtatctggacgtcgtgggctaccccttcatgcctccttattggggcctgggcttccacctgtgcaggtggggatacagcagcaccgccatcaccagacaggtggtggaaaacatgacccgggctcacttcccactggacgtgcagtggaacgacctggactacatggacagcagacgggacttcaccttcaacaaggacggcttcagagacttccccgccatggtgcaagaactgcaccaaggcggcagacggtacatgatgattgtggacccagccatcagctctagcggccctgccggaagctacagaccttacgatgagggcctgagaagaggcgtgttcatcaccaacgagacaggccagcctctgatcggcaaagtgtggcctggcagcacagcctttccagacttcacaaaccccaccgctctggcttggtgggaagatatggtggccgagttccacgatcaggtgcccttcgatggcatgtggatcgacatgaacgagcccagcaacttcatccggggcagcgaggacggctgccccaacaacgaactggaaaatcctccttacgtgcccggcgttgtcggcggaacacttcaggccgctacaatctgtgccagcagccaccagttcctcagcacccactacaacctgcacaacctgtacggcctgaccgaggccattgcctctcatagagccctggttaaggccagaggcacccggccttttgtgatcagcagaagcacattcgccggccacggcagatacgccggacattggacaggcgacgtgtggtctagttgggagcagctggctagcagcgtgccagagatcctgcagttcaatctgctgggcgtgccactcgtgggagccgacgtttgtggcttcctgggcaacaccagcgaggaactgtgtgtgcgttggacacagctgggcgccttctatcccttcatgagaaaccacaacagcctgctgagcctgcctcaagagccctacagctttagcgagcctgcacagcaggccatgagaaaggccctgactctgagatacgctctgctgccccacctgtacaccctgtttcaccaggctcacgtggccggggagacagtggctagacctctgttcctggaatttcccaaggacagctccacctggaccgtggatcatcagctgctgtggggagaagccctgctcatcacacctgttctgcaggccggaaaggccgaagtgaccggctattttcctctcggcacttggtacgacctgcagaccgtgcctattgaggccctgggatctcttcctccacctcctgctgctcctagagagcctgccatccactctgaaggccagtgggttacactgcccgctcctctggacaccatcaacgtgcacctgagagctggctacatcatcccactgcaaggccctggcctgaccacaaccgaatctagacagcagcccatggctctggccgtggctcttacaaaaggcggagaggctagaggcgagctgttctgggacgacggcgagtctctggaagtgctggaacggggcgcttatacccaagtgatcttcctggccagaaacaacaccatcgtgaacgaactcgtgcgcgtgaccagtgaaggtgctggactgcaactgcagaaagtgaccgtgctcggagtggccacagctcctcaacaggtgctgtctaacggcgtgcccgtgtccaacttcacatacagccccgacaccaaggtcctggacatctgtgtgtcactgctgatgggcgagcagttcctggtgtcctggtgc

SEQ ID NO: 15 - protein, 883 aa, Artificial Sequence
  Synthetic Construct
AHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 16 - DNA, 2304 bp, Artificial sequence
  Engineered DNA for hGAA780I 123-890
  CDS (1)..(2304)
ggacagccctggtgcttcttcccaccatcttaccccagctacaagctggaaaacctgagcagcagcgagatgggctacaccgccacactgaccagaaccacacctacattcttcccgaaggacatcctgacactgcggctggacgtgatgatggaaaccgagaaccggctgcacttcaccatcaaggaccccgccaatcggagatacgaggtgccactggaaacccctcacgtgcactctagagccccatctccactgtacagcgtggaattttccgaggaacccttcggcgtgatcgtgcggagacagctggatggaagagtgctgctgaacaccacagtggcccctctgttcttcgccgaccagtttctgcagctgagcaccagcctgcctagccagtatatcacaggcctggccgagcacctgtctccactgatgctgagcacatcctggaccagaatcaccctgtggaacagagatctggcccctacacctggcgccaacctgtacggctctcaccccttttatctggccctggaagacggcggatctgcccacggtgtttttctgctgaactccaacgccatggacgtggtgctgcagccatctcctgctctgtcttggagaagcacaggcggcatcctggacgtgtacatctttctgggccccgagcctaagagcgtggtgcagcagtatctggacgtcgtgggctaccccttcatgcctccttattggggcctgggcttccacctgtgcaggtggggatacagcagcaccgccatcaccagacaggtggtggaaaacatgacccgggctcacttcccactggacgtgcagtggaacgacctggactacatggacagcagacgggacttcaccttcaacaaggacggcttcagagacttccccgccatggtgcaagaactgcaccaaggcggcagacggtacatgatgattgtggacccagccatcagctctagcggccctgccggaagctacagaccttacgatgagggcctgagaagaggcgtgttcatcaccaacgagacaggccagcctctgatcggcaaagtgtggcctggcagcacagcctttccagacttcacaaaccccaccgctctggcttggtgggaagatatggtggccgagttccacgatcaggtgcccttcgatggcatgtggatcgacatgaacgagcccagcaacttcatccggggcagcgaggacggctgccccaacaacgaactggaaaatcctccttacgtgcccggcgttgtcggcggaacacttcaggccgctacaatctgtgccagcagccaccagttcctcagcacccactacaacctgcacaacctgtacggcctgaccgaggccattgcctctcatagagccctggttaaggccagaggcacccggccttttgtgatcagcagaagcacattcgccggccacggcagatacgccggacattggacaggcgacgtgtggtctagttgggagcagctggctagcagcgtgccagagatcctgcagttcaatctgctgggcgtgccactcgtgggagccgacgtttgtggcttcctgggcaacaccagcgaggaactgtgtgtgcgttggacacagctgggcgccttctatcccttcatgagaaaccacaacagcctgctgagcctgcctcaagagccctacagctttagcgagcctgcacagcaggccatgagaaaggccctgactctgagatacgctctgctgccccacctgtacaccctgtttcaccaggctcacgtggccggggagacagtggctagacctctgttcctggaatttcccaaggacagctccacctggaccgtggatcatcagctgctgtggggagaagccctgctcatcacacctgttctgcaggccggaaaggccgaagtgaccggctattttcctctcggcacttggtacgacctgcagaccgtgcctattgaggccctgggatctcttcctccacctcctgctgctcctagagagcctgccatccactctgaaggccagtgggttacactgcccgctcctctggacaccatcaacgtgcacctgagagctggctacatcatcccactgcaaggccctggcctgaccacaaccgaatctagacagcagcccatggctctggccgtggctcttacaaaaggcggagaggctagaggcgagctgttctgggacgacggcgagtctctggaagtgctggaacggggcgcttatacccaagtgatcttcctggccagaaacaacaccatcgtgaacgaactcgtg

SEQ ID NO: 17 - protein, 768 aa, Artificial sequence
  Synthetic Construct
GQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELV

SEQ ID NO: 18 - DNA, 2247 bp, Artificial sequence
  Engineered hGAA 70kD cDNA
  CDS (1)..(2247)
gccccatctccactgtacagcgtggaattttccgaggaacccttcggcgtgatcgtgcggagacagctggatggaagagtgctgctgaacaccacagtggcccctctgttcttcgccgaccagtttctgcagctgagcaccagcctgcctagccagtatatcacaggcctggccgagcacctgtctccactgatgctgagcacatcctggaccagaatcaccctgtggaacagagatctggcccctacacctggcgccaacctgtacggctctcaccccttttatctggccctggaagacggcggatctgcccacggtgtttttctgctgaactccaacgccatggacgtggtgctgcagccatctcctgctctgtcttggagaagcacaggcggcatcctggacgtgtacatctttctgggccccgagcctaagagcgtggtgcagcagtatctggacgtcgtgggctaccccttcatgcctccttattggggcctgggcttccacctgtgcaggtggggatacagcagcaccgccatcaccagacaggtggtggaaaacatgacccgggctcacttcccactggacgtgcagtggaacgacctggactacatggacagcagacgggacttcaccttcaacaaggacggcttcagagacttccccgccatggtgcaagaactgcaccaaggcggcagacggtacatgatgattgtggacccagccatcagctctagcggccctgccggaagctacagaccttacgatgagggcctgagaagaggcgtgttcatcaccaacgagacaggccagcctctgatcggcaaagtgtggcctggcagcacagcctttccagacttcacaaaccccaccgctctggcttggtgggaagatatggtggccgagttccacgatcaggtgcccttcgatggcatgtggatcgacatgaacgagcccagcaacttcatccggggcagcgaggacggctgccccaacaacgaactggaaaatcctccttacgtgcccggcgttgtcggcggaacacttcaggccgctacaatctgtgccagcagccaccagttcctcagcacccactacaacctgcacaacctgtacggcctgaccgaggccattgcctctcatagagccctggttaaggccagaggcacccggccttttgtgatcagcagaagcacattcgccggccacggcagatacgccggacattggacaggcgacgtgtggtctagttgggagcagctggctagcagcgtgccagagatcctgcagttcaatctgctgggcgtgccactcgtgggagccgacgtttgtggcttcctgggcaacaccagcgaggaactgtgtgtgcgttggacacagctgggcgccttctatcccttcatgagaaaccacaacagcctgctgagcctgcctcaagagccctacagctttagcgagcctgcacagcaggccatgagaaaggccctgactctgagatacgctctgctgccccacctgtacaccctgtttcaccaggctcacgtggccggggagacagtggctagacctctgttcctggaatttcccaaggacagctccacctggaccgtggatcatcagctgctgtggggagaagccctgctcatcacacctgttctgcaggccggaaaggccgaagtgaccggctattttcctctcggcacttggtacgacctgcagaccgtgcctattgaggccctgggatctcttcctccacctcctgctgctcctagagagcctgccatccactctgaaggccagtgggttacactgcccgctcctctggacaccatcaacgtgcacctgagagctggctacatcatcccactgcaaggccctggcctgaccacaaccgaatctagacagcagcccatggctctggccgtggctcttacaaaaggcggagaggctagaggcgagctgttctgggacgacggcgagtctctggaagtgctggaacggggcgcttatacccaagtgatcttcctggccagaaacaacaccatcgtgaacgaactcgtgcgcgtgaccagtgaaggtgctggactgcaactgcagaaagtgaccgtgctcggagtggccacagctcctcaacaggtgctgtctaacggcgtgcccgtgtccaacttcacatacagccccgacaccaaggtcctggacatctgtgtgtcactgctgatgggcgagcagttcctggtgtcctggtgc

SEQ ID NO: 19 - protein, 749 aa, Artificial sequence
  Synthetic Construct
APSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 20 - DNA, 2490 bp, Artificial Sequence
  Engineered DNA for hGAAV780I 76 kD protein
  CDS (1)..(2490)
ggacagccctggtgcttcttcccaccatcttaccccagctacaagctggaaaacctgagcagcagcgagatgggctacaccgccacactgaccagaaccacacctacattcttcccgaaggacatcctgacactgcggctggacgtgatgatggaaaccgagaaccggctgcacttcaccatcaaggaccccgccaatcggagatacgaggtgccactggaaacccctcacgtgcactctagagccccatctccactgtacagcgtggaattttccgaggaacccttcggcgtgatcgtgcggagacagctggatggaagagtgctgctgaacaccacagtggcccctctgttcttcgccgaccagtttctgcagctgagcaccagcctgcctagccagtatatcacaggcctggccgagcacctgtctccactgatgctgagcacatcctggaccagaatcaccctgtggaacagagatctggcccctacacctggcgccaacctgtacggctctcaccccttttatctggccctggaagacggcggatctgcccacggtgtttttctgctgaactccaacgccatggacgtggtgctgcagccatctcctgctctgtcttggagaagcacaggcggcatcctggacgtgtacatctttctgggccccgagcctaagagcgtggtgcagcagtatctggacgtcgtgggctaccccttcatgcctccttattggggcctgggcttccacctgtgcaggtggggatacagcagcaccgccatcaccagacaggtggtggaaaacatgacccgggctcacttcccactggacgtgcagtggaacgacctggactacatggacagcagacgggacttcaccttcaacaaggacggcttcagagacttccccgccatggtgcaagaactgcaccaaggcggcagacggtacatgatgattgtggacccagccatcagctctagcggccctgccggaagctacagaccttacgatgagggcctgagaagaggcgtgttcatcaccaacgagacaggccagcctctgatcggcaaagtgtggcctggcagcacagcctttccagacttcacaaaccccaccgctctggcttggtgggaagatatggtggccgagttccacgatcaggtgcccttcgatggcatgtggatcgacatgaacgagcccagcaacttcatccggggcagcgaggacggctgccccaacaacgaactggaaaatcctccttacgtgcccggcgttgtcggcggaacacttcaggccgctacaatctgtgccagcagccaccagttcctcagcacccactacaacctgcacaacctgtacggcctgaccgaggccattgcctctcatagagccctggttaaggccagaggcacccggccttttgtgatcagcagaagcacattcgccggccacggcagatacgccggacattggacaggcgacgtgtggtctagttgggagcagctggctagcagcgtgccagagatcctgcagttcaatctgctgggcgtgccactcgtgggagccgacgtttgtggcttcctgggcaacaccagcgaggaactgtgtgtgcgttggacacagctgggcgccttctatcccttcatgagaaaccacaacagcctgctgagcctgcctcaagagccctacagctttagcgagcctgcacagcaggccatgagaaaggccctgactctgagatacgctctgctgccccacctgtacaccctgtttcaccaggctcacgtggccggggagacagtggctagacctctgttcctggaatttcccaaggacagctccacctggaccgtggatcatcagctgctgtggggagaagccctgctcatcacacctgttctgcaggccggaaaggccgaagtgaccggctattttcctctcggcacttggtacgacctgcagaccgtgcctattgaggccctgggatctcttcctccacctcctgctgctcctagagagcctgccatccactctgaaggccagtgggttacactgcccgctcctctggacaccatcaacgtgcacctgagagctggctacatcatcccactgcaaggccctggcctgaccacaaccgaatctagacagcagcccatggctctggccgtggctcttacaaaaggcggagaggctagaggcgagctgttctgggacgacggcgagtctctggaagtgctggaacggggcgcttatacccaagtgatcttcctggccagaaacaacaccatcgtgaacgaactcgtgcgcgtgaccagtgaaggtgctggactgcaactgcagaaagtgaccgtgctcggagtggccacagctcctcaacaggtgctgtctaacggcgtgcccgtgtccaacttcacatacagccccgacaccaaggtcctggacatctgtgtgtcactgctgatgggcgagcagttcctggtgtcctggtgc

SEQ ID NO: 21 - protein, 830 aa, Artificial Sequence
  Synthetic Construct
GQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 22 - DNA, 2952 bp, Artificial sequence; identical to SEQ ID NO: 7
  synthetic construct
  CDS (1)..(2952)
  misc_feature (1)..(270): BiP signal peptide + vIGF2 + 2GS extension
  misc_feature (271)..(2952): engineered DNA for hGAA 61 - 952 780I
  misc_feature (2428)..(2430): Ile codon

SEQ ID NO: 23 - protein, 982 aa, Artificial sequence; identical to SEQ ID NO: 6
  Synthetic Construct

SEQ ID NO: 24 - DNA, 2952 bp, Artificial sequence; identical to SEQ ID NO: 7
  synthetic construct
  CDS (1)..(2952)
  misc_feature (1)..(270): BiP-vIGF peptide
  misc_feature (1)..(270): BiP signal peptide + vIGF2+2GS extension
  misc_feature (271)..(2952): hGAA 61-952 V780 DNA
  misc_feature (2428)..(2430): codon for hGAA 780 Valine

SEQ ID NO: 25 - protein, 982 aa, Artificial sequence; identical to SEQ ID NO: 6
  Synthetic Construct

SEQ ID NO: 26 - DNA, 22 bp, Artificial Sequence
  miRNA target sequence
agtgaattctaccagtgccata

SEQ ID NO: 27 - DNA, 24 bp, Artificial Sequence
  miRNA target sequence
agtgtgagttctaccattgccaaa

SEQ ID NO: 28 - DNA, 4581 bp, Artificial Sequence
  synthetic construct
  misc_feature (1)..(130): 5′ ITR
  enhancer (195)..(437): CMV IE Enhancer
  promoter (440)..(721): chicken beta-actin promoter
  Intron (721)..(1128): hybrid intron in CAG
  CDS (1141)..(4092): BiP-vIGF2-hGAAco
  misc_feature (3568)..(3570): Ile codon
  polyA_signal (4161)..(4287): rabbit beta-globin poly a
  misc_feature (4452)..(4581): 3′ ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcgtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccgccaccatgaagctgtctctggtggctgctatgctgctgctcctgtctgccgccagagccagcagaacactttgtggcggagagctggtggacaccctgcagtttgtgtgtggcgacagaggcttcctgttcagcagacctgccagccgggtttccagacggtctagaggaatcgtggaagagtgctgcttcagaagctgcgatctggccctgctggaaacctactgtgccacaccagccagatctgaaggcggcggaggatctggcggaggcggatctagacctggacctagagacgcccaggctcaccctggtagacctagagctgtgcctacacagtgcgacgtgccacctaacagcagattcgactgcgcccctgacaaggccatcacacaagagcagtgtgaagccagaggctgctgctacatccctgccaaacaaggactgcagggcgcccagatgggacagccttggtgcttcttcccaccatcttaccccagctacaagctggaaaacctgagcagctccgagatgggctacaccgccacactgaccagaaccacacctacattcttcccgaaggacatcctgacactgcggctggacgtgatgatggaaaccgagaaccggctgcacttcaccatcaaggaccccgccaatcggagatacgaggtgcccctggaaacaccccacgtgcactctagagcaccctctccactgtacagcgtggaattttccgaggaacccttcggcgtgatcgtgcggagacagctggatggcagagtgctcctgaataccacagtggcccctctgttcttcgccgaccagtttctgcagctgagcaccagcctgcctagccagtatatcacaggcctggccgagcatctgagccctctgatgctgagcacatcctggaccagaatcaccctgtggaaccgcgacctggctcctacacctggcgccaatctgtacggctctcaccccttctacctggcactggaagacggtggatctgcccacggtgtctttctgctgaatagcaacgccatggacgtggtgctgcagccctctcctgcactgtcttggagatctacaggcggcatcctggacgtgtacatctttctgggccccgagcctaagagcgtggtgcagcagtatctggacgtcgtgggctaccccttcatgcctccttattggggcctgggcttccacctgtgtaggtggggctacagcagcaccgccatcaccagacaggtggtggaaaacatgacccgggctcacttcccactggacgtgcagtggaacgacctggactacatggacagcagacgggacttcaccttcaacaaggacggcttcagagacttccccgccatggtgcaagagctgcatcaaggcggacggcggtacatgatgattgtggaccctgccatcagcagctctggaccagccggcagctacagaccttacgatgagggactgagaagaggcgtgttcatcaccaacgagacaggccagcctctgatcggcaaagtgtggcctggcagcacagcctttccagacttcacaaaccccaccgctctggcttggtgggaagatatggtggccgagttccacgatcaggtgcccttcgatggcatgtggatcgacatgaacgagcccagcaacttcatccggggcagcgaggatggctgccccaacaacgaactagaaaatcctccttacgtgcccggcgttgtcggcggaacacttcaggccgctacaatctgtgccagcagccatcagtttctgagcacccactacaacctgcacaacctgtacggcctgaccgaggccattgcctctcatagagccctggttaaggccagaggcacccggccttttgtgatcagcagaagcacattcgccggccacggcagatacgcaggacattggacaggcgacgtgtggtctagttgggagcagctggctagcagcgtgccagagatcctgcagttcaatctgctgggcgtgccactcgtgggagccgacgtttgtggcttcctgggcaacaccagcgaggaactgtgtgtgcgttggacacagctgggcgccttctatcccttcatgagaaaccacaacagcctgctgagcctgcctcaagagccctacagctttagcgagcctgcacagcaggccatgagaaaggccctgactctgagatacgccctgctgcctcacctgtacaccctgtttcatcaggcccacgtggcaggcgagacagtggctagacctctgttcctggaatttcccaaggacagctccacctggaccgtggatcatcagctgctgtggggagaagccctgctgattacaccagtgctgcaggccggaaaggccgaagtgacaggctatttccctctcggcacttggtacgacctgcagaccgtgcctatcgaggctctgggatctcttcctccacctcctgccgctcctagagagcctgccattcactctgaaggccagtgggttaccctgcctgctcctctggacaccatcaacgtgcacctgagagccggctacatcatccctctgcaaggccctggcctgaccacaaccgaatctagacagcagcccatggcactggccgtggctcttacaaaaggcggagaggctagaggcgagctgttctgggatgatggcgagagcctagaagtgctggaacggggcgcttatacccaagtgatcttcctggccagaaacaacaccatcgtgaacgaactcgtgcgcgtgaccagtgaaggtgctggactgcaactgcagaaagtgaccgtgctcggagtggccacagctcctcagcaggttctgtctaatggcgtgcccgtgtccaacttcacatacagccccgacaccaaggtcctggacatctgtgtgtccctgcttatgggcgagcagttcctggtgtcctggtgctgataagcggccgcacgcgtggtacctctagagtcgacccgggcggcctcgaggacggggtgaactacgcctgagatctttttccctctgccaaaaattatggggacatcatgaagccccttgagcatctgacttctggctaataaaggaaatttattttcattgcaatagtgtgttggaattttttgtgtctctcactcggttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 29 - protein, 982 aa, Artificial Sequence; identical to SEQ ID NO: 6
  Synthetic Construct

SEQ ID NO: 30 - DNA, 4687 bp, Artificial Sequence
  synthetic construct
  misc_feature (1)..(130): 5′ ITR
  enhancer (195)..(437): CMV IE Enhancer
  promoter (440)..(721): chicken beta-actin promoter
  Intron (721)..(1128): Hybrid intron in CAG
  CDS (1141)..(4092): BiP-vIGF2-hGAAco
  misc_feature (3568)..(3570): Ile codon
  misc_feature (4113)..(4134): miR-183 target
  misc_feature (4139)..(4160): miR-183 target
  misc_feature (4167)..(4188): miR-183 target
  misc_feature (4195)..(4216): miR-183 target
  polyA_signal (4267)..(4393): rabbit beta-globin poly a
  misc_feature (4558)..(4687): 3′ ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcgtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccgccaccatgaagctgtctctggtggctgctatgctgctgctcctgtctgccgccagagccagcagaacactttgtggcggagagctggtggacaccctgcagtttgtgtgtggcgacagaggcttcctgttcagcagacctgccagccgggtttccagacggtctagaggaatcgtggaagagtgctgcttcagaagctgcgatctggccctgctggaaacctactgtgccacaccagccagatctgaaggcggcggaggatctggcggaggcggatctagacctggacctagagacgcccaggctcaccctggtagacctagagctgtgcctacacagtgcgacgtgccacctaacagcagattcgactgcgcccctgacaaggccatcacacaagagcagtgtgaagccagaggctgctgctacatccctgccaaacaaggactgcagggcgcccagatgggacagccttggtgcttcttcccaccatcttaccccagctacaagctggaaaacctgagcagctccgagatgggctacaccgccacactgaccagaaccacacctacattcttcccgaaggacatcctgacactgcggctggacgtgatgatggaaaccgagaaccggctgcacttcaccatcaaggaccccgccaatcggagatacgaggtgcccctggaaacaccccacgtgcactctagagcaccctctccactgtacagcgtggaattttccgaggaacccttcggcgtgatcgtgcggagacagctggatggcagagtgctcctgaataccacagtggcccctctgttcttcgccgaccagtttctgcagctgagcaccagcctgcctagccagtatatcacaggcctggccgagcatctgagccctctgatgctgagcacatcctggaccagaatcaccctgtggaaccgcgacctggctcctacacctggcgccaatctgtacggctctcaccccttctacctggcactggaagacggtggatctgcccacggtgtctttctgctgaatagcaacgccatggacgtggtgctgcagccctctcctgcactgtcttggagatctacaggcggcatcctggacgtgtacatctttctgggccccgagcctaagagcgtggtgcagcagtatctggacgtcgtgggctaccccttcatgcctccttattggggcctgggcttccacctgtgtaggtggggctacagcagcaccgccatcaccagacaggtggtggaaaacatgacccgggctcacttcccactggacgtgcagtggaacgacctggactacatggacagcagacgggacttcaccttcaacaaggacggcttcagagacttccccgccatggtgcaagagctgcatcaaggcggacggcggtacatgatgattgtggaccctgccatcagcagctctggaccagccggcagctacagaccttacgatgagggactgagaagaggcgtgttcatcaccaacgagacaggccagcctctgatcggcaaagtgtggcctggcagcacagcctttccagacttcacaaaccccaccgctctggcttggtgggaagatatggtggccgagttccacgatcaggtgcccttcgatggcatgtggatcgacatgaacgagcccagcaacttcatccggggcagcgaggatggctgccccaacaacgaactagaaaatcctccttacgtgcccggcgttgtcggcggaacacttcaggccgctacaatctgtgccagcagccatcagtttctgagcacccactacaacctgcacaacctgtacggcctgaccgaggccattgcctctcatagagccctggttaaggccagaggcacccggccttttgtgatcagcagaagcacattcgccggccacggcagatacgcaggacattggacaggcgacgtgtggtctagttgggagcagctggctagcagcgtgccagagatcctgcagttcaatctgctgggcgtgccactcgtgggagccgacgtttgtggcttcctgggcaacaccagcgaggaactgtgtgtgcgttggacacagctgggcgccttctatcccttcatgagaaaccacaacagcctgctgagcctgcctcaagagccctacagctttagcgagcctgcacagcaggccatgagaaaggccctgactctgagatacgccctgctgcctcacctgtacaccctgtttcatcaggcccacgtggcaggcgagacagtggctagacctctgttcctggaatttcccaaggacagctccacctggaccgtggatcatcagctgctgtggggagaagccctgctgattacaccagtgctgcaggccggaaaggccgaagtgacaggctatttccctctcggcacttggtacgacctgcagaccgtgcctatcgaggctctgggatctcttcctccacctcctgccgctcctagagagcctgccattcactctgaaggccagtgggttaccctgcctgctcctctggacaccatcaacgtgcacctgagagccggctacatcatccctctgcaaggccctggcctgaccacaaccgaatctagacagcagcccatggcactggccgtggctcttacaaaaggcggagaggctagaggcgagctgttctgggatgatggcgagagcctagaagtgctggaacggggcgcttatacccaagtgatcttcctggccagaaacaacaccatcgtgaacgaactcgtgcgcgtgaccagtgaaggtgctggactgcaactgcagaaagtgaccgtgctcggagtggccacagctcctcagcaggttctgtctaatggcgtgcccgtgtccaacttcacatacagccccgacaccaaggtcctggacatctgtgtgtccctgcttatgggcgagcagttcctggtgtcctggtgctgataagcggccgcacgcgtggtaccagtgaattctaccagtgccataggatagtgaattctaccagtgccatacacgtgagtgaattctaccagtgccatagcatgcagtgaattctaccagtgccatagcggccgcctcgacccgggcggcctcgaggacggggtgaactacgcctgagatctttttccctctgccaaaaattatggggacatcatgaagccccttgagcatctgacttctggctaataaaggaaatttattttcattgcaatagtgtgttggaattttttgtgtctctcactcggttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 31 - protein, 982 aa, Artificial Sequence; identical to SEQ ID NO: 6
  Synthetic Construct

SEQ ID NO: 32 - protein, 67 aa, Homo sapiens
AYRPSETLCGGELVDTLQFVCGDRGFYFSRPASRVSRRSRGIVEECCFRSCDLALLETYCATPAKSE

SEQ ID NO: 33 - protein, 67 aa, Artificial Sequence; identical to SEQ ID NO: 32
  IGF2 F26S

SEQ ID NO: 34 - protein, 67 aa, Artificial Sequence; identical to SEQ ID NO: 32
  IGF2 Y27L

SEQ ID NO: 35 - protein, 67 aa, Artificial Sequence
  V43L
AYRPSETLCGGELVDTLQFVCGDRGFYFSRPASRVSRRSRGILEECCFRSCDLALLETYCATPAKSE

SEQ ID NO: 36 - protein, 67 aa, Artificial Sequence
  IGF2 F48T
AYRPSETLCGGELVDTLQFVCGDRGFYFSRPASRVSRRSRGIVEECCTRSCDLALLETYCATPAKSE

SEQ ID NO: 37 - protein, 67 aa, Artificial Sequence
  IGF2 R49S
AYRPSETLCGGELVDTLQFVCGDRGFYFSRPASRVSRRSRGIVEECCFSSCDLALLETYCATPAKSE

SEQ ID NO: 38 - protein, 67 aa, Artificial Sequence
  IGF2 S50I
AYRPSETLCGGELVDTLQFVCGDRGFYFSRPASRVSRRSRGIVEECCFRICDLALLETYCATPAKSE

SEQ ID NO: 39 - protein, 67 aa, Artificial Sequence
  IGF2 A54R
AYRPSETLCGGELVDTLQFVCGDRGFYFSRPASRVSRRSRGIVEECCFRSCDLRLLETYCATPAKSE

SEQ ID NO: 40 - protein, 67 aa, Artificial Sequence
  IGF2 L55R
AYRPSETLCGGELVDTLQFVCGDRGFYFSRPASRVSRRSRGIVEECCFRSCDLARLETYCATPAKSE

SEQ ID NO: 41 - protein, 67 aa, Artificial Sequence
  IGF2 F26S, Y27L, V43L, F48T, R49S, S50I, A54R, L55R
AYRPSETLCGGELVDTLQFVCGDRGSLFSRPASRVSRRSRGILEECCTSICDLRRLETYCATPAKSE

SEQ ID NO: 42 - protein, 61 aa, Artificial Sequence
  IGF2 delta1-6, Y27L, K65R
TLCGGELVDTLQFVCGDRGFLFSRPASRVSRRSRGIVEECCFRSCDLALLETYCATPARSE

SEQ ID NO: 43 - protein, 60 aa, Artificial Sequence
  IGF2 delta1-7, Y27L, K65R
LCGGELVDTLQFVCGDRGFLFSRPASRVSRRSRGIVEECCFRSCDLALLETYCATPARSE

SEQ ID NO: 44 - protein, 63 aa, Artificial Sequence
  IGF2 delta1-4, E6R, Y27L, K65R
SRTLCGGELVDTLQFVCGDRGFLFSRPASRVSRRSRGIVEECCFRSCDLALLETYCATPARSE

SEQ ID NO: 45 - protein, 63 aa, Artificial Sequence
  IGF2 delta1-4, E6R, Y27L
SRTLCGGELVDTLQFVCGDRGFLFSRPASRVSRRSRGIVEECCFRSCDLALLETYCATPAKSE

SEQ ID NO: 46 - protein, 67 aa, Artificial Sequence
  IGF2 E6R
AYRPSRTLCGGELVDTLQFVCGDRGFYFSRPASRVSRRSRGIVEECCFRSCDLALLETYCATPAKSE

SEQ ID NO: 47 - DNA, 201 bp, Homo sapiens
gcttaccgccccagtgagaccctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgag

SEQ ID NO: 48 - DNA, 189 bp, Artificial Sequence
  vIGF2 delta1-4, E6R, Y27L, K65R
tctagaacactgtgcggaggggagcttgtagacactcttcagttcgtgtgtggagatcgcgggttcctcttctctcgccccgcttccagagtttcacggaggtctaggggtatagtagaggagtgttgtttcaggtcctgtgacttggcgctcctcgagacctattgcgcgacgccagccaggtccgaa

SEQ ID NO: 49 - protein, 18 aa, Homo sapiens
MKLSLVAAMLLLLSAARA

SEQ ID NO: 50 - protein, 28 aa, Artificial Sequence
  Modified BiP-1
MKLSLVAAMLLLLSLVAAMLLLLSAARA

SEQ ID NO: 51 - protein, 25 aa, Artificial Sequence
  Modified BiP-2
MKLSLVAAMLLLLWVALLLLSAARA

SEQ ID NO: 52 - protein, 26 aa, Artificial Sequence
  Modified BiP-3
MKLSLVAAMLLLLSLVALLLLSAARA

SEQ ID NO: 53 - protein, 26 aa, Artificial Sequence
  Modified BiP-4
MKLSLVAAMLLLLALVALLLLSAARA

SEQ ID NO: 54 - protein, 17 aa, Gaussia princeps
MGVKVLFALICIAVAEA

SEQ ID NO: 55 - protein, 9 aa, Artificial Sequence
  linker sequence
GGGGSGGGG

SEQ ID NO: 56 - protein, 5 aa, Artificial Sequence
  linker sequence
GGGGS

SEQ ID NO: 57 - protein, 9 aa, Artificial Sequence
  linker sequence
GGGSGGGGS

SEQ ID NO: 58 - protein, 9 aa, Artificial Sequence
  linker sequence
GGGGSGGGS

SEQ ID NO: 59 - protein, 9 aa, Artificial Sequence
  linker sequence
GGSGSGSTS

SEQ ID NO: 60 - protein, 10 aa, Artificial Sequence
  linker sequence
GGGGSGGGGS

SEQ ID NO: 61 - protein, 6 aa, Homo sapiens
WIDMNE

SEQ ID NO: 62 - protein, 7 aa, Homo sapiens
TVPIEAL

SEQ ID NO: 63 - protein, 9 aa, Homo sapiens
QTVPIEALG

SEQ ID NO: 64 - protein, 25 aa, Homo sapiens
PLGTWYDLQTVPIEALGSLPPPPAA